SEQUENCE LISTING (31 sequences)

SEQ ID NO: 1 - DNA, 15573 bp, unidentified
  source 1..15573: mol_type = unassigned DNA; note = respiratory syncytial virus
acgcgaaaaaatgcgtacaacaaacttgcataaaccaaaaaaatggggcaaataagaatttgataagtaccacttaaatttaactcccttgcttagcgatggtgagcgagctgattaaggagaacatgcacatgaagctgtacatggagggcaccgtgaacaaccaccacttcaagtgcacatccgagggcgaaggcaagccctacgagggcacccagaccatgagaatcaaggcggtcgagggcggccctctccccttcgccttcgacatcctggctaccagcttcatgtacggcagcaaaaccttcatcaaccacacccagggcatccccgacttctttaagcagtccttccccgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagaccctgtaccccgctgacggcggcctggaaggcagagccgacatggccctgaagctcgtgggcgggggccacctgatctgcaacttgaagaccacatacagatccaagaaacccgctaagaacctcaagatgcccggcgtctactatgtggacagaagactggaaagaatcaaggaggccgacaaagagacctacgtcgagcagcacgaggtggctgtggccagatactgcgacctccctagcaaactggggcacagatgagtattcaattatagttattaaaaacttaacagaagacaaaaatggggcaaataagaatttgataagtaccacttaaatttaactcccttgcttagcgatgggttcgaattcgctatcgatgataaaagtacgtctacaaaatctatttgataatgatgaagtagcgctactaaaaataacgtgttatacggataaactaatacatctaacgaatgcgctagcgaaagcggtaatacatacgataaaactaaatggtatagtatttgtacatgtaataacgtcgtcggatatatgtccgaataataatatagtagtaaaatcgaattttacgacgatgccggtactacaaaatggtggttatatatgggaaatgatggaactaacgcattgttcgcaaccgaatggtctactagatgataattgtgaaataaaattttcgaaaaaactatcggattcgacgatgacgaattatatgaatcaactatcggaactactaggttttgatctaaatccgtaaattataattaatatcaactagcaaatcaatgtcactaacaccattagttaatataaaacttaacagaagacaaaaatggggcaaataaatcaattcagccaacccaaccatggatacgacgcataatgataatacgccgcaacgtctaatgataacggatatgcgtccgctatcgctagaaacgataataacgtcgctaacgcgtgatataataacgcataaatttatatatctaataaatcatgaatgtatagtacgtaaactagatgaacgtcaagcgacgtttacgtttctagtaaattatgaaatgaaactactacataaagtaggttcgacgaaatataaaaaatatacggaatataatacgaaatatggtacgtttccgatgccgatatttataaatcatgatggttttctagaatgtataggtataaaaccgacgaaacatacgccgataatatataaatatgatctaaatccgtaaatttcaacacaatattcacacaatctaaaacaacaactctatgcataactatactccatagtccagatggagcctgaaaattatagtaatttaaaattaaggagagatataagatagaagatggggcaaatacaaagatggctcttagcaaagtcaagttgaatgatacactcaacaaagatcaacttctgtcatccagcaaatacaccatccaacggagcacaggagatagtattgatactcctaattatgatgtgcagaaacacatcaataagttatgtggcatgttattaatcacagaagatgctaatcataaattcactgggttaataggtatgttatatgcgatgtctaggttaggaagagaagacaccataaaaatactcagagatgcgggatatcatgtaaaagcaaatggagtagatgtaacaacacatcgtcaagacattaatggaaaagaaatgaaatttgaagtgttaacattggcaagcttaacaactgaaattcaaatcaacattgagatagaatctagaaaatcctacaaaaaaatgctaaaagaaatgggagaggtagctccagaatacaggcatgactctcctgattgtgggatgataatattatgtatagcagcattagtaataactaaattagcagcaggggacagatctggtcttacagccgtgattaggagagctaataatgtcctaaaaaatgaaatgaaacgttacaaaggcttactacccaaggacatagccaacagcttctatgaagtgtttgaaaaacatccccactttatagatgtttttgttcattttggtatagcacaatcttctaccagaggtggcagtagagttgaagggatttttgcaggattgtttatgaatgcctatggtgcagggcaagtgatgttacggtggggagtcttagcaaaatcagttaaaaatattatgttaggacatgctagtgtgcaagcagaaatggaacaagttgttgaggtttatgaatatgcccaaaaattgggtggtgaagcaggattctaccatatattgaacaacccaaaagcatcattattatctttgactcaatttcctcacttctccagtgtagtattaggcaatgctgctggcctaggcataatgggagagtacagaggtacaccgaggaatcaagatctatatgatgcagcaaaggcatatgctgaacaactcaaagaaaatggtgtgattaactacagtgtactagacttgacagcagaagaactagaggctatcaaacatcagcttaatccaaaagataatgatgtagagctttgagttaataaaaaatggggcaaataaatcatcatggaaaagtttgctcctgaattccatggagaagatgcaaacaacagggctactaaattcctagaatcaataaagggcaaattcacatcacccaaagatcccaagaaaaaagatagtatcatatctgtcaactcaatagatatagaagtaaccaaagaaagccctataacatcaaattcaactattatcaacccaacaaatgagacagatgatactgcagggaacaagcccaattatcaaagaaaacctctagtaagtttcaaagaagaccctacaccaagtgataatcccttttctaaactatacaaagaaaccatagaaacatttgataacaatgaagaagaatccagctattcatacgaagaaataaatgatcagacaaacgataatataacagcaagattagataggattgatgaaaaattaagtgaaatactaggaatgcttcacacattagtagtggcaagtgcaggacctacatctgctcgggatggtataagagatgccatgattggtttaagagaagaaatgatagaaaaaatcagaactgaagcattaatgaccaatgacagattagaagctatggcaagactcaggaatgaggaaagtgaaaagatggcaaaagacacatcagatgaagtgtctctcaatccaacatcagagaaattgaacaacctattggaagggaatgatagtgacaatgatctatcacttgaagatttctgattagttaccactcttcacatcaacacacaataccaacagaagaccaacaaactaaccaacccaatcatccaaccaaacatccatccgccaatcagccaaacagccaacaaaacaaccagccaatccaaaactaaccacccggaaaaaatctataatatagttacaaaaaaaggaaagggtggggcaaatatggaaacatacgtgaacaagcttcacgaaggctccacatacacagctgctgttcaatacaatgtcttagaaaaagacgatgaccctgcatcacttacaatatgggtgcccatgttccaatcatctatgccagcagatttacttataaaagaactagctaatgtcaacatactagtgaaacaaatatccacacccaagggaccttcactaagagtcatgataaactcaagaagtgcagtgctagcacaaatgcccagcaaatttaccatatgcgctaatgtgtccttggatgaaagaagcaaactagcatatgatgtaaccacaccctgtgaaatcaaggcatgtagtctaacatgcctaaaatcaaaaaatatgttgactacagttaaagatctcactatgaagacactcaaccctacacatgatattattgctttatgtgaatttgaaaacatagtaacatcaaaaaaagtcataataccaacatacctaagatccatcagtgtcagaaataaagatctgaacacacttgaaaatataacaaccactgaattcaaaaatgctatcacaaatgcaaaaatcatcccttactcaggattactattagtcatcacagtgactgacaacaaaggagcattcaaatacataaagccacaaagtcaattcatagtagatcttggagcttacctagaaaaagaaagtatatattatgttaccacaaattggaagcacacagctacacgatttgcaatcaaacccatggaagattaacctttttcctctacatcagtgtgttaattcatacaaactttctacctacattcttcacttcaccatcacaatcacaaacactctgtggttcaaccaatcaaacaaaacttatctgaagtcccagatcatcccaagtcattgtttatcagatctagtactcaaataagttaataaaaaatatacacatggacgtccatggggcaaatgcaaacatgtccaaaaacaaggaccaacgcaccgctaagacattagaaaggacctgggacactctcaatcatttattattcatatcatcgtgcttatataagttaaatcttaaatctgtagcacaaatcacattatccattctggcaatgataatctcaacttcacttataattgcagccatcatattcatagcctcggcaaaccacaaagtcacaccaacaactgcaatcatacaagatgcaacaagccagatcaagaacacaaccccaacatacctcacccagaatcctcagcttggaatcagtccctctaatccgtctgaaattacatcacaaatcaccaccatactagcttcaacaacaccaggagtcaagtcaaccctgcaatccacaacagtcaagaccaaaaacacaacaacaactcaaacacaacccagcaagcccaccacaaaacaacgccaaaacaaaccaccaagcaaacccaataatgattttcactttgaagtgttcaactttgtaccctgcagcatatgcagcaacaatccaacctgctgggctatctgcaaaagaataccaaacaaaaaaccaggaaagaaaaccactaccaagcccacaaaaaaaccaaccctcaagacaaccaaaaaagatcccaaacctcaaaccactaaatcaaaggaagtacccaccaccaagcccacagaagagccaaccatcaacaccaccaaaacaaacatcataactacactactcacctccaacaccacaggaaatccagaactcacaagtcaaatggaaaccttccactcaacttcctccgaaggcaatccaagcccttctcaagtctctacaacatccgagtacccatcacaaccttcatctccacccaacacaccacgccagtagttacttaaaaacatattatcacaaaaggccttgaccaaccgcggagaatcaaaataaactctggggcaaataacaatggagttgccaatcctcaaagcaaatgcaattaccacaatcctcgctgcagtcacattttgctttgcttctagtcaaaacatcactgaagaattttatcaatcaacatgcagtgcagttagcaaaggctatcttagtgctctaagaactggttggtatactagtgttataactatagaattaagtaatatcaagaaaaataagtgtaatggaacagatgctaaggtaaaattgatgaaacaagaattagataaatataaaaatgctgtaacagaattgcagttgctcatgcaaagcacaccagcagcaaacaatcgagccagaagagaactaccaaggtttatgaattatacactcaacaataccaaaaaaaccaatgtaacattaagcaagaaaaggaaaagaagatttcttggttttttgttaggtgttggatctgcaatcgccagtggcattgctgtatctaaggtcctgcacttagaaggagaagtgaacaagatcaaaagtgctctactatccacaaacaaggccgtagtcagcttatcaaatggagttagtgtcttaaccagcagagtgttagacctcaaaaactatatagataaacaattgttacctattgtgaataagcaaagctgcagaatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgtgatagatacaccttgttggaaattacacacatcccctctatgtacaaccaacacaaaagaagggtcaaacatctgtttaacaagaactgacagaggatggtactgtgacaatgcaggatcagtatctttcttcccacaagctgaaaaatgtaaagttcaatcgaatcgagtattttgtgacacaatgtacagtttaacattaccaagtgaagtaaatctctgcaatgttgacatattcaatcccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgtgattatgtatcaaataaaggggtggacactgtgtctgtaggtaacacattatattatgtaaataagcaagaaggcaaaagtctctatgtaaaaggtgaaccaataataaatttctatgacccattagtattcccctctgatgaatttgatgcatcaatatctcaagtcaatgagaagattaaccagagtttagcatttattcgtaaatccgatgaattattacataatgtaaatgctggtaaatcaaccacaaatatcatgataactactataattatagtgattatagtaatattgttatcattaattgctgttggactgctcctatactgtaaggccagaagcacaccagtcacactaagcaaggatcaactgagtggtataaataatattgcatttagtaactgaataaaaatagcacctaatcatgttcttacaatggtttactatctgctcatagacaacccatctatcattggattttcttaaaatctgaacttcatcgaaactcttatctataaaccatctcacttacactatttaagtagattcctagtttatagttatataaaacacaattgaatgccagtcgaccttaccatctgtaaaaatgaaaactggggcaaatatgtcacgaaggaatccttgcaaatttgaaattcgaggtcattgcttaaatggtaagaggtgtcattttagtcataattattttgaatggccaccccatgcactgcttgtaagacaaaactttatgttaaacagaatacttaagtctatggataaaagtatagataccttatcagaaataagtggagctgcagagttggacagaacagaagagtatgctcttggtgtagttggagtgctagagagttatataggatcaataaacaatataactaaacaatcagcatgtgttgccatgagcaaactcctcactgaactcaatagtgatgatatcaaaaagctgagggacaatgaagagctaaattcacccaagataagagtgtacaatactgtcatatcatatattgaaagcaacaggaaaaacaataaacaaactatccatctgttaaaaagattgccagcagacgtattgaagaaaaccatcaaaaacacattggatatccataagagcataaccatcaacaacccaaaagaatcaactgttagtgatacaaatgaccatgccaaaaataatgatactacctgacaaatatccttgtagtataacttccatactaataacaagtagatgtagagttactatgtataatcaaaagaacacactatatttcaatcaaaacaacccaaataaccatatgtactcaccgaatcaaacattcaatgaaatccattggacctctcaagaattgattgacacaattcaaaattttctacaacatctaggtattattgaggatatatatacaatatatatattagtgtcataacactcaattctaacactcaccacatcgttacattattaattcaaacaattcaagttgtgggacaaaatggatcccattattaatggaaattctgctaatgtttatctaaccgatagttatttaaaaggtgttatctctttctcagagtgtaatgctttaggaagttacatattcaatggtccttatctcaaaaatgattataccaacttaattagtagacaaaatccattaatagaacacatgaatctaaagaaactaaatataacacagtccttaatatctaagtatcataaaggtgaaataaaattagaagaacctacttattttcagtcattacttatgacatacaagagtatgacctcgtcagaacagattgctaccactaatttacttaaaaagataataagaagagctatagaaataagtgatgtcaaagtctatgctatattgaataaactagggcttaaagaaaaggacaagattaaatccaacaatggacaagatgaagacaactcagttattacgaccataatcaaagatgatatactttcagctgttaaagataatcaatctcatcttaaagcagacaaaaatcactctacaaaacaaaaagacacaatcaaaacaacactcttgaagaaattgatgtgttcaatgcaacatcctccatcatggttaatacattggtttaacttatacacaaaattaaacaacatattaacacagtatcgatcaaatgaggtaaaaaaccatgggtttacattgatagataatcaaactcttagtggatttcaatttattttgaaccaatatggttgtatagtttatcataaggaactcaaaagaattactgtgacaacctataatcaattcttgacatggaaagatattagccttagtagattaaatgtttgtttaattacatggattagtaactgcttgaacacattaaataaaagcttaggcttaagatgcggattcaataatgttatcttgacacaactattcctttatggagattgtatactaaagctatttcacaatgaggggttctacataataaaagaggtagagggatttattatgtctctaattttaaatataacagaagaagatcaattcagaaaacgattttataatagtatgctcaacaacatcacagatgctgctaataaagctcagaaaaatctgctatcaagagtatgtcatacattattagataagacagtgtccgataatataataaatggcagatggataattctattaagtaagttccttaaattaattaagcttgcaggtgacaataaccttaacaatctgagtgaactatattttttgttcagaatatttggacacccaatggtagatgaaagacaagccatggatgctgttaaaattaattgcaatgagaccaaattttacttgttaagcagtctgagtatgttaagaggtgcctttatatatagaattataaaagggtttgtaaataattacaacagatggcctactttaagaaatgctattgttttacccttaagatggttaacttactataaactaaacacttatccttctttgttggaacttacagaaagagatttgattgtgttatcaggactacgtttctatcgtgagtttcggttgcctaaaaaagtggatcttgaaatgattataaatgataaagctatatcacctcctaaaaatttgatatggactagtttccctagaaattacatgccatcacacatacaaaactatatagaacatgaaaaattaaaattttccgagagtgataaatcaagaagagtattagagtattatttaagagataacaaattcaatgaatgtgatttatacaactgtgtagttaatcaaagttatctcaacaaccctaatcatgtggtatcattgacaggcaaagaaagagaactcagtgtaggtagaatgtttgcaatgcaaccgggaatgttcagacaggttcaaatattggcagagaaaatgatagctgaaaacattttacaattctttcctgaaagtcttacaagatatggtgatctagaactacaaaaaatattagaattgaaagcaggaataagtaacaaatcaaatcgctacaatgataattacaacaattacattagtaagtgctctatcatcacagatctcagcaaattcaatcaagcatttcgatatgaaacgtcatgtatttgtagtgatgtgctggatgaactgcatggtgtacaatctctattttcctggttacatttaactattcctcatgtcacaataatatgcacatataggcatgcacccccctatataggagatcatattgtagatcttaacaatgtagatgaacaaagtggattatatagatatcacatgggtggcatcgaagggtggtgtcaaaaactgtggaccatagaagctatatcactattggatctaatatctctcaaagggaaattctcaattactgctttaattaatggtgacaatcaatcaatagatataagcaaaccaatcagactcatggaaggtcaaactcatgctcaagcagattatttgctagcattaaatagccttaaattactgtataaagagtatgcaggcataggccacaaattaaaaggaactgagacttatatatcacgagatatgcaatttatgagtaaaacaattcaacataacggtgtatattacccagctagtataaagaaagtcctaagagtgggaccgtggataaacactatacttgatgatttcaaagtgagtctagaatctataggtagtttgacacaagaattagaatatagaggtgaaagtctattatgcagtttaatatttagaaatgtatggttatataatcagattgctctacaattaaaaaatcatgcattatgtaacaataaactatatttggacatattaaaggttctgaaacacttaaaaaccttttttaatcttgataatattgatacagcattaacattgtatatgaatttacccatgttatttggtggtggtgatcccaacttgttatatcgaagtttctatagaagaactcctgacttcctcacagaggctatagttcactctgtgttcatacttagttattatacaaaccatgacttaaaagataaacttcaagatctgtcagatgatagattgaataagttcttaacatgcataatcacgtttgacaaaaaccctaatgctgaattcgtaacattgatgagagatcctcaagctttagggtctgagagacaagctaaaattactagcgaaatcaatagactggcagttacagaggttttgagtacagctccaaacaaaatattctccaaaagtgcacaacattatactactacagagatagatctaaatgatattatgcaaaatatagaacctacatatcctcatgggctaagagttgtttatgaaagtttacccttttataaagcagagaaaatagtaaatcttatatcaggtacaaaatctataactaacatactggaaaaaacttctgccatagacttaacagatattgatagagccactgagatgatgaggaaaaacataactttgcttataaggatacttccattggattgtaacagagataaaagagagatattgagtatggaaaacctaagtattactgaattaagcaaatatgttagggaaagatcttggtctttatccaatatagttggtgttacatcacccagtatcatgtatacaatggacatcaaatatactacaagcactatatctagtggcataattatagagaaatataatgttaacagtttaacacgtggtgagagaggacccactaaaccatgggttggttcatctacacaagagaaaaaaacaatgccagtttataatagacaagtcttaaccaaaaaacagagagatcaaatagatctattagcaaaattggattgggtgtatgcatctatagataacaaggatgaattcatggaagaactcagcataggaacccttgggttaacatatgaaaaggccaagaaattatttccacaatatttaagtgtcaattatttgcatcgccttacagtcagtagtagaccatgtgaattccctgcatcaataccagcttatagaacaacaaattatcactttgacactagccctattaatcgcatattaacagaaaagtatggtgatgaagatattgacatagtattccaaaactgtataagctttggccttagtttaatgtcagtagtagaacaatttactaatgtatgtcctaacagaattattctcatacctaagcttaatgagatacatttgatgaaacctcccatattcacaggtgatgttgatattcacaagttaaaacaagtgatacaaaaacagcatatgtttttaccagacaaaataagtttgactcaatatgtggaattattcttaagtaataaaacactcaaatctggatctcatgttaattctaatttaatattggcacataaaatatctgactattttcataatacttacattttaagtactaatttagctggacattggattctgattatacaacttatgaaagattctaaaggtatttttgaaaaagattggggagagggatatataactgatcatatgtttattaatttgaaagttttcttcaatgcttataagacctatctcttgtgttttcataaaggttatggcaaagcaaagctggagtgtgatatgaacacttcagatcttctatgtgtattggaattaatagacagtagttattggaagtctatgtctaaggtatttttagaacaaaaagttatcaaatacattcttagccaagatgcaagtttacatagagtaaaaggatgtcatagcttcaaattatggtttcttaaacgtcttaatgtagcagaattcacagtttgcccttgggttgttaacatagattatcatccaacacatatgaaagcaatattaacttatatagatcttgttagaatgggattgataaatatagatagaatacacattaaaaataaacacaaattcaatgatgaattttatacttctaatctcttctacattaattataacttctcagataatactcatctattaactaaacatataaggattgctaattctgaattagaaaataattacaacaaattatatcatcctacaccagaaaccctagagaatatactagccaatccgattaaaagtaatgacaaaaagacactgaatgactattgtataggtaaaaatgttgactcaataatgttaccattgttatctaataagaagcttattaaatcgtctgcaatgattagaaccaattacagcaaacaagatttgtataatttattccctatggttgtgattgatagaattatagatcattcaggcaatacagccaaatccaaccaactttacactactacttcccaccaaatatctttagtgcacaatagcacatcactttactgcatgcttccttggcatcatattaatagattcaattttgtatttagttctacaggttgtaaaattagtatagagtatattttaaaagatcttaaaattaaagatcccaattgtatagcattcataggtgaaggagcagggaatttattattgcgtacagtagtggaacttcatcctgacataagatatatttacagaagtctgaaagattgcaatgatcatagtttacctattgagtttttaaggctgtacaatggacatatcaacattgattatggtgaaaatttgaccattcctgctacagatgcaaccaacaacattcattggtcttatttacatataaagtttgctgaacctatcagtctttttgtctgtgatgccgaattgtctgtaacagtcaactggagtaaaattataatagaatggagcaagcatgtaagaaagtgcaagtactgttcctcagttaataaatgtatgttaatagtaaaatatcatgctcaagatgatattgatttcaaattagacaatataactatattaaaaacttatgtatgcttaggcagtaagttaaagggatcggaggtttacttagtccttacaataggtcctgcgaatatattcccagtatttaatgtagtacaaaatgctaaattgatactatcaagaaccaaaaatttcatcatgcctaagaaagctgataaagagtctattgatgcaaatattaaaagtttgataccctttctttgttaccctataacaaaaaaaggaattaatactgcattgtcaaaactaaagagtgttgttagtggagatatactatcatattctatagctggacgtaatgaagttttcagcaataaacttataaatcataagcatatgaacatcttaaaatggttcaatcatgttttaaatttcagatcaacagaactaaactataaccatttatatatggtagaatctacatatccttacctaagtgaattgttaaacagcttgacaaccaatgaacttaaaaaactgattaaaatcacaggtagtctgttatacaactttcataatgaataatgaataaagatcttataataaaaattcccatagctatacactaacactgtattcaattatagttattaaaaattaaaaatcgtacgattttttaaataacttttagtgaactaatcctaaagttatcattttaatcttggaggaataaatttaaaccctaatctaattggtttatatgtgtattaactaaattacgagatattagtttttgacactttttttctcgt

SEQ ID NO: 2 - DNA, 15573 bp, unidentified
  source 1..15573: mol_type = unassigned DNA; note = respiratory syncytial virus
acgcgaaaaaatgcgtacaacaaacttgcataaaccaaaaaaatggggcaaataagaatttgataagtaccacttaaatttaactcccttgcttagcgatggtgagcgagctgattaaggagaacatgcacatgaagctgtacatggagggcaccgtgaacaaccaccacttcaagtgcacatccgagggcgaaggcaagccctacgagggcacccagaccatgagaatcaaggcggtcgagggcggccctctccccttcgccttcgacatcctggctaccagcttcatgtacggcagcaaaaccttcatcaaccacacccagggcatccccgacttctttaagcagtccttccccgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagaccctgtaccccgctgacggcggcctggaaggcagagccgacatggccctgaagctcgtgggcgggggccacctgatctgcaacttgaagaccacatacagatccaagaaacccgctaagaacctcaagatgcccggcgtctactatgtggacagaagactggaaagaatcaaggaggccgacaaagagacctacgtcgagcagcacgaggtggctgtggccagatactgcgacctccctagcaaactggggcacagatgagtattcaattatagttattaaaaacttaacagaagacaaaaatggggcaaataagaatttgataagtaccacttaaatttaactcccttgcttagcgatgggttcgaattcgctatcgatgataaaagtacgtctacaaaatctatttgataatgatgaagtagcgctactaaaaataacgtgttatacggataaactaatacatctaacgaatgcgctagcgaaagcggtaatacatacgataaaactaaatggtatagtatttgtacatgtaataacgtcgtcggatatatgtccgaataataatatagtagtaaaatcgaattttacgacgatgccggtactacaaaatggtggttatatatgggaaatgatggaactaacgcattgttcgcaaccgaatggtctactagatgataattgtgaaataaaattttcgaaaaaactatcggattcgacgatgacgaattatatgaatcaactatcggaactactaggttttgatctaaatccgtaaattataattaatatcaactagcaaatcaatgtcactaacaccattagttaatataaaacttaacagaagacaaaaatggggcaaataaatcaattcagccaacccaaccatggatacgacgcataatgataatacgccgcaacgtctaatgataacggatatgcgtccgctatcgctagaaacgataataacgtcgctaacgcgtgatataataacgcataaatttatatatctaataaatcatgaatgtatagtacgtaaactagatgaacgtcaagcgacgtttacgtttctagtaaattatgaaatgaaactactacataaagtaggttcgacgaaatataaaaaatatacggaatataatacgaaatatggtacgtttccgatgccgatatttataaatcatgatggttttctagaatgtataggtataaaaccgacgaaacatacgccgataatatataaatatgatctaaatccgtaaatttcaacacaatattcacacaatctaaaacaacaactctatgcataactatactccatagtccagatggagcctgaaaattatagtaatttaaaattaaggagagatataagatagaagatggggcaaatacaaagatggctcttagcaaagtcaagttgaatgatacactcaacaaagatcaacttctgtcatccagcaaatacaccatccaacggagcacaggagatagtattgatactcctaattatgatgtgcagaaacacatcaataagttatgtggcatgttattaatcacagaagatgctaatcataaattcactgggttaataggtatgttatatgcgatgtctaggttaggaagagaagacaccataaaaatactcagagatgcgggatatcatgtaaaagcaaatggagtagatgtaacaacacatcgtcaagacattaatggaaaagaaatgaaatttgaagtgttaacattggcaagcttaacaactgaaattcaaatcaacattgagatagaatctagaaaatcctacaaaaaaatgctaaaagaaatgggagaggtagctccagaatacaggcatgactctcctgattgtgggatgataatattatgtatagcagcattagtaataactaaattagcagcaggggacagatctggtcttacagccgtgattaggagagctaataatgtcctaaaaaatgaaatgaaacgttacaaaggcttactacccaaggacatagccaacagcttctatgaagtgtttgaaaaacatccccactttatagatgtttttgttcattttggtatagcacaatcttctaccagaggtggcagtagagttgaagggatttttgcaggattgtttatgaatgcctatggtgcagggcaagtgatgttacggtggggagtcttagcaaaatcagttaaaaatattatgttaggacatgctagtgtgcaagcagaaatggaacaagttgttgaggtttatgaatatgcccaaaaattgggtggtgaagcaggattctaccatatattgaacaacccaaaagcatcattattatctttgactcaatttcctcacttctccagtgtagtattaggcaatgctgctggcctaggcataatgggagagtacagaggtacaccgaggaatcaagatctatatgatgcagcaaaggcatatgctgaacaactcaaagaaaatggtgtgattaactacagtgtactagacttgacagcagaagaactagaggctatcaaacatcagcttaatccaaaagataatgatgtagagctttgagttaataaaaaatggggcaaataaatcatcatggaaaagtttgctcctgaattccatggagaagatgcaaacaacagggctactaaattcctagaatcaataaagggcaaattcacatcacccaaagatcccaagaaaaaagatagtatcatatctgtcaactcaatagatatagaagtaaccaaagaaagccctataacatcaaattcaactattatcaacccaacaaatgagacagatgatactgcagggaacaagcccaattatcaaagaaaacctctagtaagtttcaaagaagaccctacaccaagtgataatcccttttctaaactatacaaagaaaccatagaaacatttgataacaatgaagaagaatccagctattcatacgaagaaataaatgatcagacaaacgataatataacagcaagattagataggattgatgaaaaattaagtgaaatactaggaatgcttcacacattagtagtggcaagtgcaggacctacatctgctcgggatggtataagagatgccatgattggtttaagagaagaaatgatagaaaaaatcagaactgaagcattaatgaccaatgacagattagaagctatggcaagactcaggaatgaggaaagtgaaaagatggcaaaagacacatcagatgaagtgtctctcaatccaacatcagagaaattgaacaacctattggaagggaatgatagtgacaatgatctatcacttgaagatttctgattagttaccactcttcacatcaacacacaataccaacagaagaccaacaaactaaccaacccaatcatccaaccaaacatccatccgccaatcagccaaacagccaacaaaacaaccagccaatccaaaactaaccacccggaaaaaatctataatatagttacaaaaaaaggaaagggtggggcaaatatggaaacatacgtgaacaagcttcacgaaggctccacatacacagctgctgttcaatacaatgtcttagaaaaagacgatgaccctgcatcacttacaatatgggtgcccatgttccaatcatctatgccagcagatttacttataaaagaactagctaatgtcaacatactagtgaaacaaatatccacacccaagggaccttcactaagagtcatgataaactcaagaagtgcagtgctagcacaaatgcccagcaaatttaccatatgcgctaatgtgtccttggatgaaagaagcaaactagcatatgatgtaaccacaccctgtgaaatcaaggcatgtagtctaacatgcctaaaatcaaaaaatatgttgactacagttaaagatctcactatgaagacactcaaccctacacatgatattattgctttatgtgaatttgaaaacatagtaacatcaaaaaaagtcataataccaacatacctaagatccatcagtgtcagaaataaagatctgaacacacttgaaaatataacaaccactgaattcaaaaatgctatcacaaatgcaaaaatcatcccttactcaggattactattagtcatcacagtgactgacaacaaaggagcattcaaatacataaagccacaaagtcaattcatagtagatcttggagcttacctagaaaaagaaagtatatattatgttaccacaaattggaagcacacagctacacgatttgcaatcaaacccatggaagattaacctttttcctctacatcagtgtgttaattcatacaaactttctacctacattcttcacttcaccatcacaatcacaaacactctgtggttcaaccaatcaaacaaaacttatctgaagtcccagatcatcccaagtcattgtttatcagatctagtactcaaataagttaataaaaaatatacacatggacgtccatggggcaaatgcaaacatgtcgaaaaacaaagaccaacgtaccgcgaagacgttagaacgtacctgggatactctaaatcatttactattcatatcgtcgtgcctatataagctaaatcttaaatcggtagcacaaataacactatccatactggcgataataatctcgacttcgcttataatagcagcgatcatatttatagcctcggcgaaccataaagtcacgccaacgactgcgatcatacaagatgcgacatcgcagataaagaatacaacgccaacgtacctaacccaaaatcctcaacttggtatctcgccctcgaatccgtctgaaataacatcgcaaatcacgaccatactagcgtcaacgacaccgggagtaaagtcgaccctacaatccacgacagtaaagacgaaaaacacgacaacgactcaaacgcaaccctcgaagccgaccacgaaacaacgccaaaataaaccaccgagcaaaccgaataatgattttcactttgaagtattcaattttgtaccctgtagcatatgtagcaataatccaacgtgctgggcgatctgtaaaagaataccgaacaaaaaaccgggaaaaaaaaccacgaccaaacccacgaaaaaaccaacgctcaaaacaacgaaaaaagatcccaaaccgcaaaccacgaaatcaaaagaagtacccacgaccaaacccacggaagagccgaccataaacacgaccaaaacgaacataataactacgctactcacgtccaataccacgggaaatccggaactcacgagtcaaatggaaacgtttcactcgacttcgtccgaaggtaatccatcgccttcgcaagtctcgacaacgtccgaatacccgtcacaaccgtcatcgccaccgaacacgccacgtcagtagttacttaaaaacatattatcacaaaaggccttgaccaaccgcggagaatcaaaataaactctggggcaaataacaatggagttgccaatcctcaaagcaaatgcaattaccacaatcctcgctgcagtcacattttgctttgcttctagtcaaaacatcactgaagaattttatcaatcaacatgcagtgcagttagcaaaggctatcttagtgctctaagaactggttggtatactagtgttataactatagaattaagtaatatcaagaaaaataagtgtaatggaacagatgctaaggtaaaattgatgaaacaagaattagataaatataaaaatgctgtaacagaattgcagttgctcatgcaaagcacaccagcagcaaacaatcgagccagaagagaactaccaaggtttatgaattatacactcaacaataccaaaaaaaccaatgtaacattaagcaagaaaaggaaaagaagatttcttggttttttgttaggtgttggatctgcaatcgccagtggcattgctgtatctaaggtcctgcacttagaaggagaagtgaacaagatcaaaagtgctctactatccacaaacaaggccgtagtcagcttatcaaatggagttagtgtcttaaccagcagagtgttagacctcaaaaactatatagataaacaattgttacctattgtgaataagcaaagctgcagaatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgtgatagatacaccttgttggaaattacacacatcccctctatgtacaaccaacacaaaagaagggtcaaacatctgtttaacaagaactgacagaggatggtactgtgacaatgcaggatcagtatctttcttcccacaagctgaaaaatgtaaagttcaatcgaatcgagtattttgtgacacaatgtacagtttaacattaccaagtgaagtaaatctctgcaatgttgacatattcaatcccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgtgattatgtatcaaataaaggggtggacactgtgtctgtaggtaacacattatattatgtaaataagcaagaaggcaaaagtctctatgtaaaaggtgaaccaataataaatttctatgacccattagtattcccctctgatgaatttgatgcatcaatatctcaagtcaatgagaagattaaccagagtttagcatttattcgtaaatccgatgaattattacataatgtaaatgctggtaaatcaaccacaaatatcatgataactactataattatagtgattatagtaatattgttatcattaattgctgttggactgctcctatactgtaaggccagaagcacaccagtcacactaagcaaggatcaactgagtggtataaataatattgcatttagtaactgaataaaaatagcacctaatcatgttcttacaatggtttactatctgctcatagacaacccatctatcattggattttcttaaaatctgaacttcatcgaaactcttatctataaaccatctcacttacactatttaagtagattcctagtttatagttatataaaacacaattgaatgccagtcgaccttaccatctgtaaaaatgaaaactggggcaaatatgtcacgaaggaatccttgcaaatttgaaattcgaggtcattgcttaaatggtaagaggtgtcattttagtcataattattttgaatggccaccccatgcactgcttgtaagacaaaactttatgttaaacagaatacttaagtctatggataaaagtatagataccttatcagaaataagtggagctgcagagttggacagaacagaagagtatgctcttggtgtagttggagtgctagagagttatataggatcaataaacaatataactaaacaatcagcatgtgttgccatgagcaaactcctcactgaactcaatagtgatgatatcaaaaagctgagggacaatgaagagctaaattcacccaagataagagtgtacaatactgtcatatcatatattgaaagcaacaggaaaaacaataaacaaactatccatctgttaaaaagattgccagcagacgtattgaagaaaaccatcaaaaacacattggatatccataagagcataaccatcaacaacccaaaagaatcaactgttagtgatacaaatgaccatgccaaaaataatgatactacctgacaaatatccttgtagtataacttccatactaataacaagtagatgtagagttactatgtataatcaaaagaacacactatatttcaatcaaaacaacccaaataaccatatgtactcaccgaatcaaacattcaatgaaatccattggacctctcaagaattgattgacacaattcaaaattttctacaacatctaggtattattgaggatatatatacaatatatatattagtgtcataacactcaattctaacactcaccacatcgttacattattaattcaaacaattcaagttgtgggacaaaatggatcccattattaatggaaattctgctaatgtttatctaaccgatagttatttaaaaggtgttatctctttctcagagtgtaatgctttaggaagttacatattcaatggtccttatctcaaaaatgattataccaacttaattagtagacaaaatccattaatagaacacatgaatctaaagaaactaaatataacacagtccttaatatctaagtatcataaaggtgaaataaaattagaagaacctacttattttcagtcattacttatgacatacaagagtatgacctcgtcagaacagattgctaccactaatttacttaaaaagataataagaagagctatagaaataagtgatgtcaaagtctatgctatattgaataaactagggcttaaagaaaaggacaagattaaatccaacaatggacaagatgaagacaactcagttattacgaccataatcaaagatgatatactttcagctgttaaagataatcaatctcatcttaaagcagacaaaaatcactctacaaaacaaaaagacacaatcaaaacaacactcttgaagaaattgatgtgttcaatgcaacatcctccatcatggttaatacattggtttaacttatacacaaaattaaacaacatattaacacagtatcgatcaaatgaggtaaaaaaccatgggtttacattgatagataatcaaactcttagtggatttcaatttattttgaaccaatatggttgtatagtttatcataaggaactcaaaagaattactgtgacaacctataatcaattcttgacatggaaagatattagccttagtagattaaatgtttgtttaattacatggattagtaactgcttgaacacattaaataaaagcttaggcttaagatgcggattcaataatgttatcttgacacaactattcctttatggagattgtatactaaagctatttcacaatgaggggttctacataataaaagaggtagagggatttattatgtctctaattttaaatataacagaagaagatcaattcagaaaacgattttataatagtatgctcaacaacatcacagatgctgctaataaagctcagaaaaatctgctatcaagagtatgtcatacattattagataagacagtgtccgataatataataaatggcagatggataattctattaagtaagttccttaaattaattaagcttgcaggtgacaataaccttaacaatctgagtgaactatattttttgttcagaatatttggacacccaatggtagatgaaagacaagccatggatgctgttaaaattaattgcaatgagaccaaattttacttgttaagcagtctgagtatgttaagaggtgcctttatatatagaattataaaagggtttgtaaataattacaacagatggcctactttaagaaatgctattgttttacccttaagatggttaacttactataaactaaacacttatccttctttgttggaacttacagaaagagatttgattgtgttatcaggactacgtttctatcgtgagtttcggttgcctaaaaaagtggatcttgaaatgattataaatgataaagctatatcacctcctaaaaatttgatatggactagtttccctagaaattacatgccatcacacatacaaaactatatagaacatgaaaaattaaaattttccgagagtgataaatcaagaagagtattagagtattatttaagagataacaaattcaatgaatgtgatttatacaactgtgtagttaatcaaagttatctcaacaaccctaatcatgtggtatcattgacaggcaaagaaagagaactcagtgtaggtagaatgtttgcaatgcaaccgggaatgttcagacaggttcaaatattggcagagaaaatgatagctgaaaacattttacaattctttcctgaaagtcttacaagatatggtgatctagaactacaaaaaatattagaattgaaagcaggaataagtaacaaatcaaatcgctacaatgataattacaacaattacattagtaagtgctctatcatcacagatctcagcaaattcaatcaagcatttcgatatgaaacgtcatgtatttgtagtgatgtgctggatgaactgcatggtgtacaatctctattttcctggttacatttaactattcctcatgtcacaataatatgcacatataggcatgcacccccctatataggagatcatattgtagatcttaacaatgtagatgaacaaagtggattatatagatatcacatgggtggcatcgaagggtggtgtcaaaaactgtggaccatagaagctatatcactattggatctaatatctctcaaagggaaattctcaattactgctttaattaatggtgacaatcaatcaatagatataagcaaaccaatcagactcatggaaggtcaaactcatgctcaagcagattatttgctagcattaaatagccttaaattactgtataaagagtatgcaggcataggccacaaattaaaaggaactgagacttatatatcacgagatatgcaatttatgagtaaaacaattcaacataacggtgtatattacccagctagtataaagaaagtcctaagagtgggaccgtggataaacactatacttgatgatttcaaagtgagtctagaatctataggtagtttgacacaagaattagaatatagaggtgaaagtctattatgcagtttaatatttagaaatgtatggttatataatcagattgctctacaattaaaaaatcatgcattatgtaacaataaactatatttggacatattaaaggttctgaaacacttaaaaaccttttttaatcttgataatattgatacagcattaacattgtatatgaatttacccatgttatttggtggtggtgatcccaacttgttatatcgaagtttctatagaagaactcctgacttcctcacagaggctatagttcactctgtgttcatacttagttattatacaaaccatgacttaaaagataaacttcaagatctgtcagatgatagattgaataagttcttaacatgcataatcacgtttgacaaaaaccctaatgctgaattcgtaacattgatgagagatcctcaagctttagggtctgagagacaagctaaaattactagcgaaatcaatagactggcagttacagaggttttgagtacagctccaaacaaaatattctccaaaagtgcacaacattatactactacagagatagatctaaatgatattatgcaaaatatagaacctacatatcctcatgggctaagagttgtttatgaaagtttacccttttataaagcagagaaaatagtaaatcttatatcaggtacaaaatctataactaacatactggaaaaaacttctgccatagacttaacagatattgatagagccactgagatgatgaggaaaaacataactttgcttataaggatacttccattggattgtaacagagataaaagagagatattgagtatggaaaacctaagtattactgaattaagcaaatatgttagggaaagatcttggtctttatccaatatagttggtgttacatcacccagtatcatgtatacaatggacatcaaatatactacaagcactatatctagtggcataattatagagaaatataatgttaacagtttaacacgtggtgagagaggacccactaaaccatgggttggttcatctacacaagagaaaaaaacaatgccagtttataatagacaagtcttaaccaaaaaacagagagatcaaatagatctattagcaaaattggattgggtgtatgcatctatagataacaaggatgaattcatggaagaactcagcataggaacccttgggttaacatatgaaaaggccaagaaattatttccacaatatttaagtgtcaattatttgcatcgccttacagtcagtagtagaccatgtgaattccctgcatcaataccagcttatagaacaacaaattatcactttgacactagccctattaatcgcatattaacagaaaagtatggtgatgaagatattgacatagtattccaaaactgtataagctttggccttagtttaatgtcagtagtagaacaatttactaatgtatgtcctaacagaattattctcatacctaagcttaatgagatacatttgatgaaacctcccatattcacaggtgatgttgatattcacaagttaaaacaagtgatacaaaaacagcatatgtttttaccagacaaaataagtttgactcaatatgtggaattattcttaagtaataaaacactcaaatctggatctcatgttaattctaatttaatattggcacataaaatatctgactattttcataatacttacattttaagtactaatttagctggacattggattctgattatacaacttatgaaagattctaaaggtatttttgaaaaagattggggagagggatatataactgatcatatgtttattaatttgaaagttttcttcaatgcttataagacctatctcttgtgttttcataaaggttatggcaaagcaaagctggagtgtgatatgaacacttcagatcttctatgtgtattggaattaatagacagtagttattggaagtctatgtctaaggtatttttagaacaaaaagttatcaaatacattcttagccaagatgcaagtttacatagagtaaaaggatgtcatagcttcaaattatggtttcttaaacgtcttaatgtagcagaattcacagtttgcccttgggttgttaacatagattatcatccaacacatatgaaagcaatattaacttatatagatcttgttagaatgggattgataaatatagatagaatacacattaaaaataaacacaaattcaatgatgaattttatacttctaatctcttctacattaattataacttctcagataatactcatctattaactaaacatataaggattgctaattctgaattagaaaataattacaacaaattatatcatcctacaccagaaaccctagagaatatactagccaatccgattaaaagtaatgacaaaaagacactgaatgactattgtataggtaaaaatgttgactcaataatgttaccattgttatctaataagaagcttattaaatcgtctgcaatgattagaaccaattacagcaaacaagatttgtataatttattccctatggttgtgattgatagaattatagatcattcaggcaatacagccaaatccaaccaactttacactactacttcccaccaaatatctttagtgcacaatagcacatcactttactgcatgcttccttggcatcatattaatagattcaattttgtatttagttctacaggttgtaaaattagtatagagtatattttaaaagatcttaaaattaaagatcccaattgtatagcattcataggtgaaggagcagggaatttattattgcgtacagtagtggaacttcatcctgacataagatatatttacagaagtctgaaagattgcaatgatcatagtttacctattgagtttttaaggctgtacaatggacatatcaacattgattatggtgaaaatttgaccattcctgctacagatgcaaccaacaacattcattggtcttatttacatataaagtttgctgaacctatcagtctttttgtctgtgatgccgaattgtctgtaacagtcaactggagtaaaattataatagaatggagcaagcatgtaagaaagtgcaagtactgttcctcagttaataaatgtatgttaatagtaaaatatcatgctcaagatgatattgatttcaaattagacaatataactatattaaaaacttatgtatgcttaggcagtaagttaaagggatcggaggtttacttagtccttacaataggtcctgcgaatatattcccagtatttaatgtagtacaaaatgctaaattgatactatcaagaaccaaaaatttcatcatgcctaagaaagctgataaagagtctattgatgcaaatattaaaagtttgataccctttctttgttaccctataacaaaaaaaggaattaatactgcattgtcaaaactaaagagtgttgttagtggagatatactatcatattctatagctggacgtaatgaagttttcagcaataaacttataaatcataagcatatgaacatcttaaaatggttcaatcatgttttaaatttcagatcaacagaactaaactataaccatttatatatggtagaatctacatatccttacctaagtgaattgttaaacagcttgacaaccaatgaacttaaaaaactgattaaaatcacaggtagtctgttatacaactttcataatgaataatgaataaagatcttataataaaaattcccatagctatacactaacactgtattcaattatagttattaaaaattaaaaatcgtacgattttttaaataacttttagtgaactaatcctaaagttatcattttaatcttggaggaataaatttaaaccctaatctaattggtttatatgtgtattaactaaattacgagatattagtttttgacactttttttctcgt

SEQ ID NO: 3 - DNA, 15573 bp, unidentified
  source 1..15573: mol_type = unassigned DNA; note = respiratory syncytial virus
acgcgaaaaaatgcgtacaacaaacttgcataaaccaaaaaaatggggcaaataagaatttgataagtaccacttaaatttaactcccttgcttagcgatggtgagcgagctgattaaggagaacatgcacatgaagctgtacatggagggcaccgtgaacaaccaccacttcaagtgcacatccgagggcgaaggcaagccctacgagggcacccagaccatgagaatcaaggcggtcgagggcggccctctccccttcgccttcgacatcctggctaccagcttcatgtacggcagcaaaaccttcatcaaccacacccagggcatccccgacttctttaagcagtccttccccgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagaccctgtaccccgctgacggcggcctggaaggcagagccgacatggccctgaagctcgtgggcgggggccacctgatctgcaacttgaagaccacatacagatccaagaaacccgctaagaacctcaagatgcccggcgtctactatgtggacagaagactggaaagaatcaaggaggccgacaaagagacctacgtcgagcagcacgaggtggctgtggccagatactgcgacctccctagcaaactggggcacagatgagtattcaattatagttattaaaaacttaacagaagacaaaaatggggcaaataagaatttgataagtaccacttaaatttaactcccttgcttagcgatgggttcgaattcgctatcgatgataaaagtacgtctacaaaatctatttgataatgatgaagtagcgctactaaaaataacgtgttatacggataaactaatacatctaacgaatgcgctagcgaaagcggtaatacatacgataaaactaaatggtatagtatttgtacatgtaataacgtcgtcggatatatgtccgaataataatatagtagtaaaatcgaattttacgacgatgccggtactacaaaatggtggttatatatgggaaatgatggaactaacgcattgttcgcaaccgaatggtctactagatgataattgtgaaataaaattttcgaaaaaactatcggattcgacgatgacgaattatatgaatcaactatcggaactactaggttttgatctaaatccgtaaattataattaatatcaactagcaaatcaatgtcactaacaccattagttaatataaaacttaacagaagacaaaaatggggcaaataaatcaattcagccaacccaaccatggatacgacgcataatgataatacgccgcaacgtctaatgataacggatatgcgtccgctatcgctagaaacgataataacgtcgctaacgcgtgatataataacgcataaatttatatatctaataaatcatgaatgtatagtacgtaaactagatgaacgtcaagcgacgtttacgtttctagtaaattatgaaatgaaactactacataaagtaggttcgacgaaatataaaaaatatacggaatataatacgaaatatggtacgtttccgatgccgatatttataaatcatgatggttttctagaatgtataggtataaaaccgacgaaacatacgccgataatatataaatatgatctaaatccgtaaatttcaacacaatattcacacaatctaaaacaacaactctatgcataactatactccatagtccagatggagcctgaaaattatagtaatttaaaattaaggagagatataagatagaagatggggcaaatacaaagatggctcttagcaaagtcaagttgaatgatacactcaacaaagatcaacttctgtcatccagcaaatacaccatccaacggagcacaggagatagtattgatactcctaattatgatgtgcagaaacacatcaataagttatgtggcatgttattaatcacagaagatgctaatcataaattcactgggttaataggtatgttatatgcgatgtctaggttaggaagagaagacaccataaaaatactcagagatgcgggatatcatgtaaaagcaaatggagtagatgtaacaacacatcgtcaagacattaatggaaaagaaatgaaatttgaagtgttaacattggcaagcttaacaactgaaattcaaatcaacattgagatagaatctagaaaatcctacaaaaaaatgctaaaagaaatgggagaggtagctccagaatacaggcatgactctcctgattgtgggatgataatattatgtatagcagcattagtaataactaaattagcagcaggggacagatctggtcttacagccgtgattaggagagctaataatgtcctaaaaaatgaaatgaaacgttacaaaggcttactacccaaggacatagccaacagcttctatgaagtgtttgaaaaacatccccactttatagatgtttttgttcattttggtatagcacaatcttctaccagaggtggcagtagagttgaagggatttttgcaggattgtttatgaatgcctatggtgcagggcaagtgatgttacggtggggagtcttagcaaaatcagttaaaaatattatgttaggacatgctagtgtgcaagcagaaatggaacaagttgttgaggtttatgaatatgcccaaaaattgggtggtgaagcaggattctaccatatattgaacaacccaaaagcatcattattatctttgactcaatttcctcacttctccagtgtagtattaggcaatgctgctggcctaggcataatgggagagtacagaggtacaccgaggaatcaagatctatatgatgcagcaaaggcatatgctgaacaactcaaagaaaatggtgtgattaactacagtgtactagacttgacagcagaagaactagaggctatcaaacatcagcttaatccaaaagataatgatgtagagctttgagttaataaaaaatggggcaaataaatcatcatggaaaagtttgctcctgaattccatggagaagatgcaaacaacagggctactaaattcctagaatcaataaagggcaaattcacatcacccaaagatcccaagaaaaaagatagtatcatatctgtcaactcaatagatatagaagtaaccaaagaaagccctataacatcaaattcaactattatcaacccaacaaatgagacagatgatactgcagggaacaagcccaattatcaaagaaaacctctagtaagtttcaaagaagaccctacaccaagtgataatcccttttctaaactatacaaagaaaccatagaaacatttgataacaatgaagaagaatccagctattcatacgaagaaataaatgatcagacaaacgataatataacagcaagattagataggattgatgaaaaattaagtgaaatactaggaatgcttcacacattagtagtggcaagtgcaggacctacatctgctcgggatggtataagagatgccatgattggtttaagagaagaaatgatagaaaaaatcagaactgaagcattaatgaccaatgacagattagaagctatggcaagactcaggaatgaggaaagtgaaaagatggcaaaagacacatcagatgaagtgtctctcaatccaacatcagagaaattgaacaacctattggaagggaatgatagtgacaatgatctatcacttgaagatttctgattagttaccactcttcacatcaacacacaataccaacagaagaccaacaaactaaccaacccaatcatccaaccaaacatccatccgccaatcagccaaacagccaacaaaacaaccagccaatccaaaactaaccacccggaaaaaatctataatatagttacaaaaaaaggaaagggtggggcaaatatggaaacatacgtgaacaagcttcacgaaggctccacatacacagctgctgttcaatacaatgtcttagaaaaagacgatgaccctgcatcacttacaatatgggtgcccatgttccaatcatctatgccagcagatttacttataaaagaactagctaatgtcaacatactagtgaaacaaatatccacacccaagggaccttcactaagagtcatgataaactcaagaagtgcagtgctagcacaaatgcccagcaaatttaccatatgcgctaatgtgtccttggatgaaagaagcaaactagcatatgatgtaaccacaccctgtgaaatcaaggcatgtagtctaacatgcctaaaatcaaaaaatatgttgactacagttaaagatctcactatgaagacactcaaccctacacatgatattattgctttatgtgaatttgaaaacatagtaacatcaaaaaaagtcataataccaacatacctaagatccatcagtgtcagaaataaagatctgaacacacttgaaaatataacaaccactgaattcaaaaatgctatcacaaatgcaaaaatcatcccttactcaggattactattagtcatcacagtgactgacaacaaaggagcattcaaatacataaagccacaaagtcaattcatagtagatcttggagcttacctagaaaaagaaagtatatattatgttaccacaaattggaagcacacagctacacgatttgcaatcaaacccatggaagattaacctttttcctctacatcagtgtgttaattcatacaaactttctacctacattcttcacttcaccatcacaatcacaaacactctgtggttcaaccaatcaaacaaaacttatctgaagtcccagatcatcccaagtcattgtttatcagatctagtactcaaataagttaataaaaaatatacacatggacgtccatggggcaaatgcaaacatgtcgaaaaataaagaccaacgtacggcgaagacgctagaacgtacctgggatacgctaaatcatttactatttatatcgtcgtgcctatataaactaaatcttaaatcggtagcgcaaataacactatcgatactggcgataataatatcgacttcgctaataatagcagcgataatatttatagcctcggcgaatcataaagtcacgccgacgactgcgataatacaagatgcgacatcgcaaataaagaatacgacgccaacgtatctaacccaaaatccgcaacttggtatatcgccctcgaatccgtcggaaataacatcgcaaataacgaccatactagcgtcgacgacaccgggtgtaaagtcgacgctacaatccacgacggtaaagacgaaaaatacgacaacgacgcaaacgcaaccgtcgaaaccgaccacgaaacaacgtcaaaataaaccaccgtcgaaaccgaataatgattttcactttgaagtatttaattttgtaccctgttcgatatgtagcaataatccgacgtgctgggcgatatgtaaaagaataccgaataaaaaaccgggaaaaaaaacgacgaccaaaccgacgaaaaaaccaacgctaaaaacaacgaaaaaagatccgaaaccgcaaaccacgaaatcgaaagaagtacccacgacgaaacccacggaagaaccgaccataaatacgaccaaaacgaatataataactacgctactaacgtccaatacgacgggaaatccggaactaacgagtcaaatggaaacgtttcattcgacttcgtcggaaggtaatccatcgccgtcgcaagtctcgacgacttccgaatatccgtcacaaccgtcgtcgccaccgaatacgccacgtcaatagttacttaaaaacatattatcacaaaaggccttgaccaaccgcggagaatcaaaataaactctggggcaaataacaatggagttgccaatcctcaaagcaaatgcaattaccacaatcctcgctgcagtcacattttgctttgcttctagtcaaaacatcactgaagaattttatcaatcaacatgcagtgcagttagcaaaggctatcttagtgctctaagaactggttggtatactagtgttataactatagaattaagtaatatcaagaaaaataagtgtaatggaacagatgctaaggtaaaattgatgaaacaagaattagataaatataaaaatgctgtaacagaattgcagttgctcatgcaaagcacaccagcagcaaacaatcgagccagaagagaactaccaaggtttatgaattatacactcaacaataccaaaaaaaccaatgtaacattaagcaagaaaaggaaaagaagatttcttggttttttgttaggtgttggatctgcaatcgccagtggcattgctgtatctaaggtcctgcacttagaaggagaagtgaacaagatcaaaagtgctctactatccacaaacaaggccgtagtcagcttatcaaatggagttagtgtcttaaccagcagagtgttagacctcaaaaactatatagataaacaattgttacctattgtgaataagcaaagctgcagaatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgtgatagatacaccttgttggaaattacacacatcccctctatgtacaaccaacacaaaagaagggtcaaacatctgtttaacaagaactgacagaggatggtactgtgacaatgcaggatcagtatctttcttcccacaagctgaaaaatgtaaagttcaatcgaatcgagtattttgtgacacaatgtacagtttaacattaccaagtgaagtaaatctctgcaatgttgacatattcaatcccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgtgattatgtatcaaataaaggggtggacactgtgtctgtaggtaacacattatattatgtaaataagcaagaaggcaaaagtctctatgtaaaaggtgaaccaataataaatttctatgacccattagtattcccctctgatgaatttgatgcatcaatatctcaagtcaatgagaagattaaccagagtttagcatttattcgtaaatccgatgaattattacataatgtaaatgctggtaaatcaaccacaaatatcatgataactactataattatagtgattatagtaatattgttatcattaattgctgttggactgctcctatactgtaaggccagaagcacaccagtcacactaagcaaggatcaactgagtggtataaataatattgcatttagtaactgaataaaaatagcacctaatcatgttcttacaatggtttactatctgctcatagacaacccatctatcattggattttcttaaaatctgaacttcatcgaaactcttatctataaaccatctcacttacactatttaagtagattcctagtttatagttatataaaacacaattgaatgccagtcgaccttaccatctgtaaaaatgaaaactggggcaaatatgtcacgaaggaatccttgcaaatttgaaattcgaggtcattgcttaaatggtaagaggtgtcattttagtcataattattttgaatggccaccccatgcactgcttgtaagacaaaactttatgttaaacagaatacttaagtctatggataaaagtatagataccttatcagaaataagtggagctgcagagttggacagaacagaagagtatgctcttggtgtagttggagtgctagagagttatataggatcaataaacaatataactaaacaatcagcatgtgttgccatgagcaaactcctcactgaactcaatagtgatgatatcaaaaagctgagggacaatgaagagctaaattcacccaagataagagtgtacaatactgtcatatcatatattgaaagcaacaggaaaaacaataaacaaactatccatctgttaaaaagattgccagcagacgtattgaagaaaaccatcaaaaacacattggatatccataagagcataaccatcaacaacccaaaagaatcaactgttagtgatacaaatgaccatgccaaaaataatgatactacctgacaaatatccttgtagtataacttccatactaataacaagtagatgtagagttactatgtataatcaaaagaacacactatatttcaatcaaaacaacccaaataaccatatgtactcaccgaatcaaacattcaatgaaatccattggacctctcaagaattgattgacacaattcaaaattttctacaacatctaggtattattgaggatatatatacaatatatatattagtgtcataacactcaattctaacactcaccacatcgttacattattaattcaaacaattcaagttgtgggacaaaatggatcccattattaatggaaattctgctaatgtttatctaaccgatagttatttaaaaggtgttatctctttctcagagtgtaatgctttaggaagttacatattcaatggtccttatctcaaaaatgattataccaacttaattagtagacaaaatccattaatagaacacatgaatctaaagaaactaaatataacacagtccttaatatctaagtatcataaaggtgaaataaaattagaagaacctacttattttcagtcattacttatgacatacaagagtatgacctcgtcagaacagattgctaccactaatttacttaaaaagataataagaagagctatagaaataagtgatgtcaaagtctatgctatattgaataaactagggcttaaagaaaaggacaagattaaatccaacaatggacaagatgaagacaactcagttattacgaccataatcaaagatgatatactttcagctgttaaagataatcaatctcatcttaaagcagacaaaaatcactctacaaaacaaaaagacacaatcaaaacaacactcttgaagaaattgatgtgttcaatgcaacatcctccatcatggttaatacattggtttaacttatacacaaaattaaacaacatattaacacagtatcgatcaaatgaggtaaaaaaccatgggtttacattgatagataatcaaactcttagtggatttcaatttattttgaaccaatatggttgtatagtttatcataaggaactcaaaagaattactgtgacaacctataatcaattcttgacatggaaagatattagccttagtagattaaatgtttgtttaattacatggattagtaactgcttgaacacattaaataaaagcttaggcttaagatgcggattcaataatgttatcttgacacaactattcctttatggagattgtatactaaagctatttcacaatgaggggttctacataataaaagaggtagagggatttattatgtctctaattttaaatataacagaagaagatcaattcagaaaacgattttataatagtatgctcaacaacatcacagatgctgctaataaagctcagaaaaatctgctatcaagagtatgtcatacattattagataagacagtgtccgataatataataaatggcagatggataattctattaagtaagttccttaaattaattaagcttgcaggtgacaataaccttaacaatctgagtgaactatattttttgttcagaatatttggacacccaatggtagatgaaagacaagccatggatgctgttaaaattaattgcaatgagaccaaattttacttgttaagcagtctgagtatgttaagaggtgcctttatatatagaattataaaagggtttgtaaataattacaacagatggcctactttaagaaatgctattgttttacccttaagatggttaacttactataaactaaacacttatccttctttgttggaacttacagaaagagatttgattgtgttatcaggactacgtttctatcgtgagtttcggttgcctaaaaaagtggatcttgaaatgattataaatgataaagctatatcacctcctaaaaatttgatatggactagtttccctagaaattacatgccatcacacatacaaaactatatagaacatgaaaaattaaaattttccgagagtgataaatcaagaagagtattagagtattatttaagagataacaaattcaatgaatgtgatttatacaactgtgtagttaatcaaagttatctcaacaaccctaatcatgtggtatcattgacaggcaaagaaagagaactcagtgtaggtagaatgtttgcaatgcaaccgggaatgttcagacaggttcaaatattggcagagaaaatgatagctgaaaacattttacaattctttcctgaaagtcttacaagatatggtgatctagaactacaaaaaatattagaattgaaagcaggaataagtaacaaatcaaatcgctacaatgataattacaacaattacattagtaagtgctctatcatcacagatctcagcaaattcaatcaagcatttcgatatgaaacgtcatgtatttgtagtgatgtgctggatgaactgcatggtgtacaatctctattttcctggttacatttaactattcctcatgtcacaataatatgcacatataggcatgcacccccctatataggagatcatattgtagatcttaacaatgtagatgaacaaagtggattatatagatatcacatgggtggcatcgaagggtggtgtcaaaaactgtggaccatagaagctatatcactattggatctaatatctctcaaagggaaattctcaattactgctttaattaatggtgacaatcaatcaatagatataagcaaaccaatcagactcatggaaggtcaaactcatgctcaagcagattatttgctagcattaaatagccttaaattactgtataaagagtatgcaggcataggccacaaattaaaaggaactgagacttatatatcacgagatatgcaatttatgagtaaaacaattcaacataacggtgtatattacccagctagtataaagaaagtcctaagagtgggaccgtggataaacactatacttgatgatttcaaagtgagtctagaatctataggtagtttgacacaagaattagaatatagaggtgaaagtctattatgcagtttaatatttagaaatgtatggttatataatcagattgctctacaattaaaaaatcatgcattatgtaacaataaactatatttggacatattaaaggttctgaaacacttaaaaaccttttttaatcttgataatattgatacagcattaacattgtatatgaatttacccatgttatttggtggtggtgatcccaacttgttatatcgaagtttctatagaagaactcctgacttcctcacagaggctatagttcactctgtgttcatacttagttattatacaaaccatgacttaaaagataaacttcaagatctgtcagatgatagattgaataagttcttaacatgcataatcacgtttgacaaaaaccctaatgctgaattcgtaacattgatgagagatcctcaagctttagggtctgagagacaagctaaaattactagcgaaatcaatagactggcagttacagaggttttgagtacagctccaaacaaaatattctccaaaagtgcacaacattatactactacagagatagatctaaatgatattatgcaaaatatagaacctacatatcctcatgggctaagagttgtttatgaaagtttacccttttataaagcagagaaaatagtaaatcttatatcaggtacaaaatctataactaacatactggaaaaaacttctgccatagacttaacagatattgatagagccactgagatgatgaggaaaaacataactttgcttataaggatacttccattggattgtaacagagataaaagagagatattgagtatggaaaacctaagtattactgaattaagcaaatatgttagggaaagatcttggtctttatccaatatagttggtgttacatcacccagtatcatgtatacaatggacatcaaatatactacaagcactatatctagtggcataattatagagaaatataatgttaacagtttaacacgtggtgagagaggacccactaaaccatgggttggttcatctacacaagagaaaaaaacaatgccagtttataatagacaagtcttaaccaaaaaacagagagatcaaatagatctattagcaaaattggattgggtgtatgcatctatagataacaaggatgaattcatggaagaactcagcataggaacccttgggttaacatatgaaaaggccaagaaattatttccacaatatttaagtgtcaattatttgcatcgccttacagtcagtagtagaccatgtgaattccctgcatcaataccagcttatagaacaacaaattatcactttgacactagccctattaatcgcatattaacagaaaagtatggtgatgaagatattgacatagtattccaaaactgtataagctttggccttagtttaatgtcagtagtagaacaatttactaatgtatgtcctaacagaattattctcatacctaagcttaatgagatacatttgatgaaacctcccatattcacaggtgatgttgatattcacaagttaaaacaagtgatacaaaaacagcatatgtttttaccagacaaaataagtttgactcaatatgtggaattattcttaagtaataaaacactcaaatctggatctcatgttaattctaatttaatattggcacataaaatatctgactattttcataatacttacattttaagtactaatttagctggacattggattctgattatacaacttatgaaagattctaaaggtatttttgaaaaagattggggagagggatatataactgatcatatgtttattaatttgaaagttttcttcaatgcttataagacctatctcttgtgttttcataaaggttatggcaaagcaaagctggagtgtgatatgaacacttcagatcttctatgtgtattggaattaatagacagtagttattggaagtctatgtctaaggtatttttagaacaaaaagttatcaaatacattcttagccaagatgcaagtttacatagagtaaaaggatgtcatagcttcaaattatggtttcttaaacgtcttaatgtagcagaattcacagtttgcccttgggttgttaacatagattatcatccaacacatatgaaagcaatattaacttatatagatcttgttagaatgggattgataaatatagatagaatacacattaaaaataaacacaaattcaatgatgaattttatacttctaatctcttctacattaattataacttctcagataatactcatctattaactaaacatataaggattgctaattctgaattagaaaataattacaacaaattatatcatcctacaccagaaaccctagagaatatactagccaatccgattaaaagtaatgacaaaaagacactgaatgactattgtataggtaaaaatgttgactcaataatgttaccattgttatctaataagaagcttattaaatcgtctgcaatgattagaaccaattacagcaaacaagatttgtataatttattccctatggttgtgattgatagaattatagatcattcaggcaatacagccaaatccaaccaactttacactactacttcccaccaaatatctttagtgcacaatagcacatcactttactgcatgcttccttggcatcatattaatagattcaattttgtatttagttctacaggttgtaaaattagtatagagtatattttaaaagatcttaaaattaaagatcccaattgtatagcattcataggtgaaggagcagggaatttattattgcgtacagtagtggaacttcatcctgacataagatatatttacagaagtctgaaagattgcaatgatcatagtttacctattgagtttttaaggctgtacaatggacatatcaacattgattatggtgaaaatttgaccattcctgctacagatgcaaccaacaacattcattggtcttatttacatataaagtttgctgaacctatcagtctttttgtctgtgatgccgaattgtctgtaacagtcaactggagtaaaattataatagaatggagcaagcatgtaagaaagtgcaagtactgttcctcagttaataaatgtatgttaatagtaaaatatcatgctcaagatgatattgatttcaaattagacaatataactatattaaaaacttatgtatgcttaggcagtaagttaaagggatcggaggtttacttagtccttacaataggtcctgcgaatatattcccagtatttaatgtagtacaaaatgctaaattgatactatcaagaaccaaaaatttcatcatgcctaagaaagctgataaagagtctattgatgcaaatattaaaagtttgataccctttctttgttaccctataacaaaaaaaggaattaatactgcattgtcaaaactaaagagtgttgttagtggagatatactatcatattctatagctggacgtaatgaagttttcagcaataaacttataaatcataagcatatgaacatcttaaaatggttcaatcatgttttaaatttcagatcaacagaactaaactataaccatttatatatggtagaatctacatatccttacctaagtgaattgttaaacagcttgacaaccaatgaacttaaaaaactgattaaaatcacaggtagtctgttatacaactttcataatgaataatgaataaagatcttataataaaaattcccatagctatacactaacactgtattcaattatagttattaaaaattaaaaatcgtacgattttttaaataacttttagtgaactaatcctaaagttatcattttaatcttggaggaataaatttaaaccctaatctaattggtttatatgtgtattaactaaattacgagatattagtttttgacactttttttctcgt

SEQ ID NO: 4 - DNA, 15573 bp, unidentified
  source 1..15573: mol_type = unassigned DNA; note = respiratory syncytial virus
acgcgaaaaaatgcgtacaacaaacttgcataaaccaaaaaaatggggcaaataagaatttgataagtaccacttaaatttaactcccttgcttagcgatggtgagcgagctgattaaggagaacatgcacatgaagctgtacatggagggcaccgtgaacaaccaccacttcaagtgcacatccgagggcgaaggcaagccctacgagggcacccagaccatgagaatcaaggcggtcgagggcggccctctccccttcgccttcgacatcctggctaccagcttcatgtacggcagcaaaaccttcatcaaccacacccagggcatccccgacttctttaagcagtccttccccgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagaccctgtaccccgctgacggcggcctggaaggcagagccgacatggccctgaagctcgtgggcgggggccacctgatctgcaacttgaagaccacatacagatccaagaaacccgctaagaacctcaagatgcccggcgtctactatgtggacagaagactggaaagaatcaaggaggccgacaaagagacctacgtcgagcagcacgaggtggctgtggccagatactgcgacctccctagcaaactggggcacagatgagtattcaattatagttattaaaaacttaacagaagacaaaaatggggcaaataagaatttgataagtaccacttaaatttaactcccttgcttagcgatgggttcgaattcgctatcgatgataaaagtacgtctacaaaatctatttgataatgatgaagtagcgctactaaaaataacgtgttatacggataaactaatacatctaacgaatgcgctagcgaaagcggtaatacatacgataaaactaaatggtatagtatttgtacatgtaataacgtcgtcggatatatgtccgaataataatatagtagtaaaatcgaattttacgacgatgccggtactacaaaatggtggttatatatgggaaatgatggaactaacgcattgttcgcaaccgaatggtctactagatgataattgtgaaataaaattttcgaaaaaactatcggattcgacgatgacgaattatatgaatcaactatcggaactactaggttttgatctaaatccgtaaattataattaatatcaactagcaaatcaatgtcactaacaccattagttaatataaaacttaacagaagacaaaaatggggcaaataaatcaattcagccaacccaaccatggatacgacgcataatgataatacgccgcaacgtctaatgataacggatatgcgtccgctatcgctagaaacgataataacgtcgctaacgcgtgatataataacgcataaatttatatatctaataaatcatgaatgtatagtacgtaaactagatgaacgtcaagcgacgtttacgtttctagtaaattatgaaatgaaactactacataaagtaggttcgacgaaatataaaaaatatacggaatataatacgaaatatggtacgtttccgatgccgatatttataaatcatgatggttttctagaatgtataggtataaaaccgacgaaacatacgccgataatatataaatatgatctaaatccgtaaatttcaacacaatattcacacaatctaaaacaacaactctatgcataactatactccatagtccagatggagcctgaaaattatagtaatttaaaattaaggagagatataagatagaagatggggcaaatacaaagatggctcttagcaaagtcaagttgaatgatacactcaacaaagatcaacttctgtcatccagcaaatacaccatccaacggagcacaggagatagtattgatactcctaattatgatgtgcagaaacacatcaataagttatgtggcatgttattaatcacagaagatgctaatcataaattcactgggttaataggtatgttatatgcgatgtctaggttaggaagagaagacaccataaaaatactcagagatgcgggatatcatgtaaaagcaaatggagtagatgtaacaacacatcgtcaagacattaatggaaaagaaatgaaatttgaagtgttaacattggcaagcttaacaactgaaattcaaatcaacattgagatagaatctagaaaatcctacaaaaaaatgctaaaagaaatgggagaggtagctccagaatacaggcatgactctcctgattgtgggatgataatattatgtatagcagcattagtaataactaaattagcagcaggggacagatctggtcttacagccgtgattaggagagctaataatgtcctaaaaaatgaaatgaaacgttacaaaggcttactacccaaggacatagccaacagcttctatgaagtgtttgaaaaacatccccactttatagatgtttttgttcattttggtatagcacaatcttctaccagaggtggcagtagagttgaagggatttttgcaggattgtttatgaatgcctatggtgcagggcaagtgatgttacggtggggagtcttagcaaaatcagttaaaaatattatgttaggacatgctagtgtgcaagcagaaatggaacaagttgttgaggtttatgaatatgcccaaaaattgggtggtgaagcaggattctaccatatattgaacaacccaaaagcatcattattatctttgactcaatttcctcacttctccagtgtagtattaggcaatgctgctggcctaggcataatgggagagtacagaggtacaccgaggaatcaagatctatatgatgcagcaaaggcatatgctgaacaactcaaagaaaatggtgtgattaactacagtgtactagacttgacagcagaagaactagaggctatcaaacatcagcttaatccaaaagataatgatgtagagctttgagttaataaaaaatggggcaaataaatcatcatggaaaagtttgctcctgaattccatggagaagatgcaaacaacagggctactaaattcctagaatcaataaagggcaaattcacatcacccaaagatcccaagaaaaaagatagtatcatatctgtcaactcaatagatatagaagtaaccaaagaaagccctataacatcaaattcaactattatcaacccaacaaatgagacagatgatactgcagggaacaagcccaattatcaaagaaaacctctagtaagtttcaaagaagaccctacaccaagtgataatcccttttctaaactatacaaagaaaccatagaaacatttgataacaatgaagaagaatccagctattcatacgaagaaataaatgatcagacaaacgataatataacagcaagattagataggattgatgaaaaattaagtgaaatactaggaatgcttcacacattagtagtggcaagtgcaggacctacatctgctcgggatggtataagagatgccatgattggtttaagagaagaaatgatagaaaaaatcagaactgaagcattaatgaccaatgacagattagaagctatggcaagactcaggaatgaggaaagtgaaaagatggcaaaagacacatcagatgaagtgtctctcaatccaacatcagagaaattgaacaacctattggaagggaatgatagtgacaatgatctatcacttgaagatttctgattagttaccactcttcacatcaacacacaataccaacagaagaccaacaaactaaccaacccaatcatccaaccaaacatccatccgccaatcagccaaacagccaacaaaacaaccagccaatccaaaactaaccacccggaaaaaatctataatatagttacaaaaaaaggaaagggtggggcaaatatggaaacatacgtgaacaagcttcacgaaggctccacatacacagctgctgttcaatacaatgtcttagaaaaagacgatgaccctgcatcacttacaatatgggtgcccatgttccaatcatctatgccagcagatttacttataaaagaactagctaatgtcaacatactagtgaaacaaatatccacacccaagggaccttcactaagagtcatgataaactcaagaagtgcagtgctagcacaaatgcccagcaaatttaccatatgcgctaatgtgtccttggatgaaagaagcaaactagcatatgatgtaaccacaccctgtgaaatcaaggcatgtagtctaacatgcctaaaatcaaaaaatatgttgactacagttaaagatctcactatgaagacactcaaccctacacatgatattattgctttatgtgaatttgaaaacatagtaacatcaaaaaaagtcataataccaacatacctaagatccatcagtgtcagaaataaagatctgaacacacttgaaaatataacaaccactgaattcaaaaatgctatcacaaatgcaaaaatcatcccttactcaggattactattagtcatcacagtgactgacaacaaaggagcattcaaatacataaagccacaaagtcaattcatagtagatcttggagcttacctagaaaaagaaagtatatattatgttaccacaaattggaagcacacagctacacgatttgcaatcaaacccatggaagattaacctttttcctctacatcagtgtgttaattcatacaaactttctacctacattcttcacttcaccatcacaatcacaaacactctgtggttcaaccaatcaaacaaaacttatctgaagtcccagatcatcccaagtcattgtttatcagatctagtactcaaataagttaataaaaaatatacacatggacgtccatggggcaaatgcaaacatgtcgaaaaataaagatcaacgtacggcgaaaacgctagaacgtacgtgggatacgctaaatcatctactatttatatcgtcgtgtctatataaactaaatctaaaatcggtagcgcaaataacgctatcgatactagcgataataatatcgacttcgctaataatagcggcgataatatttatagcgtcggcgaatcataaagtaacgccgacgacggcgataatacaagatgcgacttcgcaaataaaaaatacgacgccgacgtatctaacgcaaaatccgcaactaggtatatcgccgtcgaatccgtcggaaataacgtcgcaaataacgacgatactagcgtcgacgacgccgggtgtaaaatcgacgctacaatcgacgacggtaaaaacgaaaaatacgacgacgacgcaaacgcaaccgtcgaaaccgacgacgaaacaacgtcaaaataaaccgccgtcgaaaccgaataatgattttcattttgaagtatttaattttgtaccgtgttcgatatgttcgaataatccgacgtgttgggcgatatgtaaacgtataccgaataaaaaaccgggtaaaaaaacgacgacgaaaccgacgaaaaaaccgacgctaaaaacgacgaaaaaagatccgaaaccgcaaacgacgaaatcgaaagaagtaccgacgacgaaaccgacggaagaaccgacgataaatacgacgaaaacgaatataataacgacgctactaacgtcgaatacgacgggtaatccggaactaacgtcgcaaatggaaacgtttcattcgacttcgtcggaaggtaatccgtcgccgtcgcaagtatcgacgacttcggaatatccgtcgcaaccgtcgtcgccgccgaatacgccgcgtcaatagttacttaaaaacatattatcacaaaaggccttgaccaaccgcggagaatcaaaataaactctggggcaaataacaatggagttgccaatcctcaaagcaaatgcaattaccacaatcctcgctgcagtcacattttgctttgcttctagtcaaaacatcactgaagaattttatcaatcaacatgcagtgcagttagcaaaggctatcttagtgctctaagaactggttggtatactagtgttataactatagaattaagtaatatcaagaaaaataagtgtaatggaacagatgctaaggtaaaattgatgaaacaagaattagataaatataaaaatgctgtaacagaattgcagttgctcatgcaaagcacaccagcagcaaacaatcgagccagaagagaactaccaaggtttatgaattatacactcaacaataccaaaaaaaccaatgtaacattaagcaagaaaaggaaaagaagatttcttggttttttgttaggtgttggatctgcaatcgccagtggcattgctgtatctaaggtcctgcacttagaaggagaagtgaacaagatcaaaagtgctctactatccacaaacaaggccgtagtcagcttatcaaatggagttagtgtcttaaccagcagagtgttagacctcaaaaactatatagataaacaattgttacctattgtgaataagcaaagctgcagaatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgtgatagatacaccttgttggaaattacacacatcccctctatgtacaaccaacacaaaagaagggtcaaacatctgtttaacaagaactgacagaggatggtactgtgacaatgcaggatcagtatctttcttcccacaagctgaaaaatgtaaagttcaatcgaatcgagtattttgtgacacaatgtacagtttaacattaccaagtgaagtaaatctctgcaatgttgacatattcaatcccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgtgattatgtatcaaataaaggggtggacactgtgtctgtaggtaacacattatattatgtaaataagcaagaaggcaaaagtctctatgtaaaaggtgaaccaataataaatttctatgacccattagtattcccctctgatgaatttgatgcatcaatatctcaagtcaatgagaagattaaccagagtttagcatttattcgtaaatccgatgaattattacataatgtaaatgctggtaaatcaaccacaaatatcatgataactactataattatagtgattatagtaatattgttatcattaattgctgttggactgctcctatactgtaaggccagaagcacaccagtcacactaagcaaggatcaactgagtggtataaataatattgcatttagtaactgaataaaaatagcacctaatcatgttcttacaatggtttactatctgctcatagacaacccatctatcattggattttcttaaaatctgaacttcatcgaaactcttatctataaaccatctcacttacactatttaagtagattcctagtttatagttatataaaacacaattgaatgccagtcgaccttaccatctgtaaaaatgaaaactggggcaaatatgtcacgaaggaatccttgcaaatttgaaattcgaggtcattgcttaaatggtaagaggtgtcattttagtcataattattttgaatggccaccccatgcactgcttgtaagacaaaactttatgttaaacagaatacttaagtctatggataaaagtatagataccttatcagaaataagtggagctgcagagttggacagaacagaagagtatgctcttggtgtagttggagtgctagagagttatataggatcaataaacaatataactaaacaatcagcatgtgttgccatgagcaaactcctcactgaactcaatagtgatgatatcaaaaagctgagggacaatgaagagctaaattcacccaagataagagtgtacaatactgtcatatcatatattgaaagcaacaggaaaaacaataaacaaactatccatctgttaaaaagattgccagcagacgtattgaagaaaaccatcaaaaacacattggatatccataagagcataaccatcaacaacccaaaagaatcaactgttagtgatacaaatgaccatgccaaaaataatgatactacctgacaaatatccttgtagtataacttccatactaataacaagtagatgtagagttactatgtataatcaaaagaacacactatatttcaatcaaaacaacccaaataaccatatgtactcaccgaatcaaacattcaatgaaatccattggacctctcaagaattgattgacacaattcaaaattttctacaacatctaggtattattgaggatatatatacaatatatatattagtgtcataacactcaattctaacactcaccacatcgttacattattaattcaaacaattcaagttgtgggacaaaatggatcccattattaatggaaattctgctaatgtttatctaaccgatagttatttaaaaggtgttatctctttctcagagtgtaatgctttaggaagttacatattcaatggtccttatctcaaaaatgattataccaacttaattagtagacaaaatccattaatagaacacatgaatctaaagaaactaaatataacacagtccttaatatctaagtatcataaaggtgaaataaaattagaagaacctacttattttcagtcattacttatgacatacaagagtatgacctcgtcagaacagattgctaccactaatttacttaaaaagataataagaagagctatagaaataagtgatgtcaaagtctatgctatattgaataaactagggcttaaagaaaaggacaagattaaatccaacaatggacaagatgaagacaactcagttattacgaccataatcaaagatgatatactttcagctgttaaagataatcaatctcatcttaaagcagacaaaaatcactctacaaaacaaaaagacacaatcaaaacaacactcttgaagaaattgatgtgttcaatgcaacatcctccatcatggttaatacattggtttaacttatacacaaaattaaacaacatattaacacagtatcgatcaaatgaggtaaaaaaccatgggtttacattgatagataatcaaactcttagtggatttcaatttattttgaaccaatatggttgtatagtttatcataaggaactcaaaagaattactgtgacaacctataatcaattcttgacatggaaagatattagccttagtagattaaatgtttgtttaattacatggattagtaactgcttgaacacattaaataaaagcttaggcttaagatgcggattcaataatgttatcttgacacaactattcctttatggagattgtatactaaagctatttcacaatgaggggttctacataataaaagaggtagagggatttattatgtctctaattttaaatataacagaagaagatcaattcagaaaacgattttataatagtatgctcaacaacatcacagatgctgctaataaagctcagaaaaatctgctatcaagagtatgtcatacattattagataagacagtgtccgataatataataaatggcagatggataattctattaagtaagttccttaaattaattaagcttgcaggtgacaataaccttaacaatctgagtgaactatattttttgttcagaatatttggacacccaatggtagatgaaagacaagccatggatgctgttaaaattaattgcaatgagaccaaattttacttgttaagcagtctgagtatgttaagaggtgcctttatatatagaattataaaagggtttgtaaataattacaacagatggcctactttaagaaatgctattgttttacccttaagatggttaacttactataaactaaacacttatccttctttgttggaacttacagaaagagatttgattgtgttatcaggactacgtttctatcgtgagtttcggttgcctaaaaaagtggatcttgaaatgattataaatgataaagctatatcacctcctaaaaatttgatatggactagtttccctagaaattacatgccatcacacatacaaaactatatagaacatgaaaaattaaaattttccgagagtgataaatcaagaagagtattagagtattatttaagagataacaaattcaatgaatgtgatttatacaactgtgtagttaatcaaagttatctcaacaaccctaatcatgtggtatcattgacaggcaaagaaagagaactcagtgtaggtagaatgtttgcaatgcaaccgggaatgttcagacaggttcaaatattggcagagaaaatgatagctgaaaacattttacaattctttcctgaaagtcttacaagatatggtgatctagaactacaaaaaatattagaattgaaagcaggaataagtaacaaatcaaatcgctacaatgataattacaacaattacattagtaagtgctctatcatcacagatctcagcaaattcaatcaagcatttcgatatgaaacgtcatgtatttgtagtgatgtgctggatgaactgcatggtgtacaatctctattttcctggttacatttaactattcctcatgtcacaataatatgcacatataggcatgcacccccctatataggagatcatattgtagatcttaacaatgtagatgaacaaagtggattatatagatatcacatgggtggcatcgaagggtggtgtcaaaaactgtggaccatagaagctatatcactattggatctaatatctctcaaagggaaattctcaattactgctttaattaatggtgacaatcaatcaatagatataagcaaaccaatcagactcatggaaggtcaaactcatgctcaagcagattatttgctagcattaaatagccttaaattactgtataaagagtatgcaggcataggccacaaattaaaaggaactgagacttatatatcacgagatatgcaatttatgagtaaaacaattcaacataacggtgtatattacccagctagtataaagaaagtcctaagagtgggaccgtggataaacactatacttgatgatttcaaagtgagtctagaatctataggtagtttgacacaagaattagaatatagaggtgaaagtctattatgcagtttaatatttagaaatgtatggttatataatcagattgctctacaattaaaaaatcatgcattatgtaacaataaactatatttggacatattaaaggttctgaaacacttaaaaaccttttttaatcttgataatattgatacagcattaacattgtatatgaatttacccatgttatttggtggtggtgatcccaacttgttatatcgaagtttctatagaagaactcctgacttcctcacagaggctatagttcactctgtgttcatacttagttattatacaaaccatgacttaaaagataaacttcaagatctgtcagatgatagattgaataagttcttaacatgcataatcacgtttgacaaaaaccctaatgctgaattcgtaacattgatgagagatcctcaagctttagggtctgagagacaagctaaaattactagcgaaatcaatagactggcagttacagaggttttgagtacagctccaaacaaaatattctccaaaagtgcacaacattatactactacagagatagatctaaatgatattatgcaaaatatagaacctacatatcctcatgggctaagagttgtttatgaaagtttacccttttataaagcagagaaaatagtaaatcttatatcaggtacaaaatctataactaacatactggaaaaaacttctgccatagacttaacagatattgatagagccactgagatgatgaggaaaaacataactttgcttataaggatacttccattggattgtaacagagataaaagagagatattgagtatggaaaacctaagtattactgaattaagcaaatatgttagggaaagatcttggtctttatccaatatagttggtgttacatcacccagtatcatgtatacaatggacatcaaatatactacaagcactatatctagtggcataattatagagaaatataatgttaacagtttaacacgtggtgagagaggacccactaaaccatgggttggttcatctacacaagagaaaaaaacaatgccagtttataatagacaagtcttaaccaaaaaacagagagatcaaatagatctattagcaaaattggattgggtgtatgcatctatagataacaaggatgaattcatggaagaactcagcataggaacccttgggttaacatatgaaaaggccaagaaattatttccacaatatttaagtgtcaattatttgcatcgccttacagtcagtagtagaccatgtgaattccctgcatcaataccagcttatagaacaacaaattatcactttgacactagccctattaatcgcatattaacagaaaagtatggtgatgaagatattgacatagtattccaaaactgtataagctttggccttagtttaatgtcagtagtagaacaatttactaatgtatgtcctaacagaattattctcatacctaagcttaatgagatacatttgatgaaacctcccatattcacaggtgatgttgatattcacaagttaaaacaagtgatacaaaaacagcatatgtttttaccagacaaaataagtttgactcaatatgtggaattattcttaagtaataaaacactcaaatctggatctcatgttaattctaatttaatattggcacataaaatatctgactattttcataatacttacattttaagtactaatttagctggacattggattctgattatacaacttatgaaagattctaaaggtatttttgaaaaagattggggagagggatatataactgatcatatgtttattaatttgaaagttttcttcaatgcttataagacctatctcttgtgttttcataaaggttatggcaaagcaaagctggagtgtgatatgaacacttcagatcttctatgtgtattggaattaatagacagtagttattggaagtctatgtctaaggtatttttagaacaaaaagttatcaaatacattcttagccaagatgcaagtttacatagagtaaaaggatgtcatagcttcaaattatggtttcttaaacgtcttaatgtagcagaattcacagtttgcccttgggttgttaacatagattatcatccaacacatatgaaagcaatattaacttatatagatcttgttagaatgggattgataaatatagatagaatacacattaaaaataaacacaaattcaatgatgaattttatacttctaatctcttctacattaattataacttctcagataatactcatctattaactaaacatataaggattgctaattctgaattagaaaataattacaacaaattatatcatcctacaccagaaaccctagagaatatactagccaatccgattaaaagtaatgacaaaaagacactgaatgactattgtataggtaaaaatgttgactcaataatgttaccattgttatctaataagaagcttattaaatcgtctgcaatgattagaaccaattacagcaaacaagatttgtataatttattccctatggttgtgattgatagaattatagatcattcaggcaatacagccaaatccaaccaactttacactactacttcccaccaaatatctttagtgcacaatagcacatcactttactgcatgcttccttggcatcatattaatagattcaattttgtatttagttctacaggttgtaaaattagtatagagtatattttaaaagatcttaaaattaaagatcccaattgtatagcattcataggtgaaggagcagggaatttattattgcgtacagtagtggaacttcatcctgacataagatatatttacagaagtctgaaagattgcaatgatcatagtttacctattgagtttttaaggctgtacaatggacatatcaacattgattatggtgaaaatttgaccattcctgctacagatgcaaccaacaacattcattggtcttatttacatataaagtttgctgaacctatcagtctttttgtctgtgatgccgaattgtctgtaacagtcaactggagtaaaattataatagaatggagcaagcatgtaagaaagtgcaagtactgttcctcagttaataaatgtatgttaatagtaaaatatcatgctcaagatgatattgatttcaaattagacaatataactatattaaaaacttatgtatgcttaggcagtaagttaaagggatcggaggtttacttagtccttacaataggtcctgcgaatatattcccagtatttaatgtagtacaaaatgctaaattgatactatcaagaaccaaaaatttcatcatgcctaagaaagctgataaagagtctattgatgcaaatattaaaagtttgataccctttctttgttaccctataacaaaaaaaggaattaatactgcattgtcaaaactaaagagtgttgttagtggagatatactatcatattctatagctggacgtaatgaagttttcagcaataaacttataaatcataagcatatgaacatcttaaaatggttcaatcatgttttaaatttcagatcaacagaactaaactataaccatttatatatggtagaatctacatatccttacctaagtgaattgttaaacagcttgacaaccaatgaacttaaaaaactgattaaaatcacaggtagtctgttatacaactttcataatgaataatgaataaagatcttataataaaaattcccatagctatacactaacactgtattcaattatagttattaaaaattaaaaatcgtacgattttttaaataacttttagtgaactaatcctaaagttatcattttaatcttggaggaataaatttaaaccctaatctaattggtttatatgtgtattaactaaattacgagatattagtttttgacactttttttctcgt

SEQ ID NO: 5 - protein, 139 aa, unidentified
  VARIANT 3: note = Any amino acid
  VARIANT 5: note = Any amino acid
  VARIANT 8: note = Any amino acid
  VARIANT 11: note = Any amino acid
  VARIANT 17..18: note = Any amino acid
  VARIANT 31: note = Any amino acid
  VARIANT 36: note = Any amino acid
  VARIANT 45: note = Any amino acid
  VARIANT 57: note = Any amino acid
  VARIANT 64..65: note = Any amino acid
  VARIANT 68: note = Any amino acid
  VARIANT 70: note = Any amino acid
  VARIANT 82: note = Any amino acid
  VARIANT 84: note = Any amino acid
  VARIANT 90: note = Any amino acid
  VARIANT 92..93: note = Any amino acid
  VARIANT 101: note = Any amino acid
  VARIANT 104..106: note = Any amino acid
  VARIANT 115..116: note = Any amino acid
  VARIANT 118: note = Any amino acid
  VARIANT 121: note = Any amino acid
  VARIANT 124: note = Any amino acid
  VARIANT 126..127: note = Any amino acid
  VARIANT 129: note = Any amino acid
  VARIANT 131: note = Any amino acid
  VARIANT 135: note = Any amino acid
  VARIANT 138..139: note = Any amino acid
  source 1..139: note = respiratory syncytial virus
MGXNXLSXIKXRLQNLXXNDEVALLKITCYXDKLIXLTNALAKAXIHTIKLNGIVFXHVITSSXXCPXNXIVVKSNFTTMPXLXNGGYIXEXXELTHCSQXNGXXXDNCEIKFSXXLXDSXMTXYXXQXSXLLGXDLXX

SEQ ID NO: 6 - DNA, 420 bp, unidentified
  source 1..420: mol_type = unassigned DNA; note = respiratory syncytial virus
atgggttcgaattcgctatcgatgataaaagtacgtctacaaaatctatttgataatgatgaagtagcgctactaaaaataacgtgttatacggataaactaatacatctaacgaatgcgctagcgaaagcggtaatacatacgataaaactaaatggtatagtatttgtacatgtaataacgtcgtcggatatatgtccgaataataatatagtagtaaaatcgaattttacgacgatgccggtactacaaaatggtggttatatatgggaaatgatggaactaacgcattgttcgcaaccgaatggtctactagatgataattgtgaaataaaattttcgaaaaaactatcggattcgacgatgacgaattatatgaatcaactatcggaactactaggttttgatctaaatccgtaa

SEQ ID NO: 7 - DNA, 420 bp, unidentified
  source 1..420: mol_type = unassigned DNA; note = respiratory syncytial virus
atggggtcgaactcgctctcgatgatcaaggtccgcctccagaatctcttcgacaacgacgaggtcgcgctcctcaagatcacgtgttacacggacaagctcatccacctcacgaacgcgctcgcgaaggcggtcatccacacgatcaagctcaacgggatcgtcttcgtccacgtcatcacgtcgtcggacatctgtccgaacaacaacatcgtcgtcaagtcgaacttcacgacgatgccggtcctccagaacggggggtacatctgggagatgatggagctcacgcactgttcgcagccgaacgggctcctcgacgacaactgtgagatcaagttctcgaagaagctctcggactcgacgatgacgaactacatgaaccagctctcggagctcctcgggttcgacctcaacccgtaa

SEQ ID NO: 8 - protein, 124 aa, unidentified
  VARIANT 2: note = Any amino acid
  VARIANT 4..8: note = Any amino acid
  VARIANT 10: note = Any amino acid
  VARIANT 12: note = Any amino acid
  VARIANT 14: note = Any amino acid
  VARIANT 21: note = Any amino acid
  VARIANT 23..26: note = Any amino acid
  VARIANT 28: note = Any amino acid
  VARIANT 32..33: note = Any amino acid
  VARIANT 38: note = Any amino acid
  VARIANT 45: note = Any amino acid
  VARIANT 50: note = Any amino acid
  VARIANT 55: note = Any amino acid
  VARIANT 59..60: note = Any amino acid
  VARIANT 68: note = Any amino acid
  VARIANT 72: note = Any amino acid
  VARIANT 76..77: note = Any amino acid
  VARIANT 81: note = Any amino acid
  VARIANT 98: note = Any amino acid
  VARIANT 100: note = Any amino acid
  VARIANT 103: note = Any amino acid
  VARIANT 108: note = Any amino acid
  VARIANT 118: note = Any amino acid
  source 1..124: note = respiratory syncytial virus
MXTXXXXXTXQXLXITDMRPXSXXXXIXSLTXXIITHXFIYLINXECIVXKLDEXQATXXFLVNYEMXLLHXVGSXXYKKXTEYNTKYGTFPMPIFIXHXGFXECIGXKPTKHTPIIXKYDLNP

SEQ ID NO: 9 - DNA, 375 bp, unidentified
  source 1..375: mol_type = unassigned DNA; note = respiratory syncytial virus
atggatacgacgcataatgataatacgccgcaacgtctaatgataacggatatgcgtccgctatcgctagaaacgataataacgtcgctaacgcgtgatataataacgcataaatttatatatctaataaatcatgaatgtatagtacgtaaactagatgaacgtcaagcgacgtttacgtttctagtaaattatgaaatgaaactactacataaagtaggttcgacgaaatataaaaaatatacggaatataatacgaaatatggtacgtttccgatgccgatatttataaatcatgatggttttctagaatgtataggtataaaaccgacgaaacatacgccgataatatataaatatgatctaaatccgtaa

SEQ ID NO: 10 - DNA, 375 bp, unidentified
  source 1..375: mol_type = unassigned DNA; note = respiratory syncytial virus
atggacacgacgcacaacgacaacacgccgcagcgcctcatgatcacggacatgcgcccgctctcgctcgagacgatcatcacgtcgctcacgcgcgacatcatcacgcacaagttcatctacctcatcaaccacgagtgtatcgtccgcaagctcgacgagcgccaggcgacgttcacgttcctcgtcaactacgagatgaagctcctccacaaggtcgggtcgacgaagtacaagaagtacacggagtacaacacgaagtacgggacgttcccgatgccgatcttcatcaaccacgacgggttcctcgagtgtatcgggatcaagccgacgaagcacacgccgatcatctacaagtacgacctcaacccgtaa

SEQ ID NO: 11 - protein, 30 aa, Homo sapiens
TTNIMITTIIIVIIVILLSLIAVGLLLYCK

SEQ ID NO: 12 - protein, 26 aa, Homo sapiens
ARSTPVPILKANAITTILAAVTFCFA

SEQ ID NO: 13 - protein, 20 aa, Homo sapiens
AVTFCFASSQNITEEFYQST

SEQ ID NO: 14 - protein, 33 aa, Homo sapiens
QSTCSAVSKGYLSALRTGWYTSVITIELSNIKK

SEQ ID NO: 15 - protein, 27 aa, Homo sapiens
IKKNKCNGTDAKVKLMKQELDKYKNAV

SEQ ID NO: 16 - protein, 33 aa, Homo sapiens
FPQAEKCKVQSNRVFCDTMYSLTLPSEVNLCNV

SEQ ID NO: 17 - protein, 574 aa, Homo sapiens
MELPILKANAITTILAAVTFCFASSQNITEEFYQSTCSAVSKGYLSALRTGWYTSVITIELSNIKKNKCNGTDAKVKLMKQELDKYKNAVTELQLLMQSTPAANNRARRELPRFMNYTLNNTKKTNVTLSKKRKRRFLGFLLGVGSAIASGIAVSKVLHLEGEVNKIKSALLSTNKAVVSLSNGVSVLTSRVLDLKNYIDKQLLPIVNKQSCRISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAEKCKVQSNRVFCDTMYSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 18 - DNA, 897 bp, unidentified
  source 1..897: mol_type = unassigned DNA; note = respiratory syncytial virus
atgtcgaaaaacaaagaccaacgtaccgcgaagacgttagaacgtacctgggatactctaaatcatttactattcatatcgtcgtgcctatataagctaaatcttaaatcggtagcacaaataacactatccatactggcgataataatctcgacttcgcttataatagcagcgatcatatttatagcctcggcgaaccataaagtcacgccaacgactgcgatcatacaagatgcgacatcgcagataaagaatacaacgccaacgtacctaacccaaaatcctcaacttggtatctcgccctcgaatccgtctgaaataacatcgcaaatcacgaccatactagcgtcaacgacaccgggagtaaagtcgaccctacaatccacgacagtaaagacgaaaaacacgacaacgactcaaacgcaaccctcgaagccgaccacgaaacaacgccaaaataaaccaccgagcaaaccgaataatgattttcactttgaagtattcaattttgtaccctgtagcatatgtagcaataatccaacgtgctgggcgatctgtaaaagaataccgaacaaaaaaccgggaaaaaaaaccacgaccaaacccacgaaaaaaccaacgctcaaaacaacgaaaaaagatcccaaaccgcaaaccacgaaatcaaaagaagtacccacgaccaaacccacggaagagccgaccataaacacgaccaaaacgaacataataactacgctactcacgtccaataccacgggaaatccggaactcacgagtcaaatggaaacgtttcactcgacttcgtccgaaggtaatccatcgccttcgcaagtctcgacaacgtccgaatacccgtcacaaccgtcatcgccaccgaacacgccacgtcagtag

SEQ ID NO: 19 - DNA, 897 bp, unidentified
  source 1..897: mol_type = unassigned DNA; note = respiratory syncytial virus
atgtcgaaaaataaagaccaacgtacggcgaagacgctagaacgtacctgggatacgctaaatcatttactatttatatcgtcgtgcctatataaactaaatcttaaatcggtagcgcaaataacactatcgatactggcgataataatatcgacttcgctaataatagcagcgataatatttatagcctcggcgaatcataaagtcacgccgacgactgcgataatacaagatgcgacatcgcaaataaagaatacgacgccaacgtatctaacccaaaatccgcaacttggtatatcgccctcgaatccgtcggaaataacatcgcaaataacgaccatactagcgtcgacgacaccgggtgtaaagtcgacgctacaatccacgacggtaaagacgaaaaatacgacaacgacgcaaacgcaaccgtcgaaaccgaccacgaaacaacgtcaaaataaaccaccgtcgaaaccgaataatgattttcactttgaagtatttaattttgtaccctgttcgatatgtagcaataatccgacgtgctgggcgatatgtaaaagaataccgaataaaaaaccgggaaaaaaaacgacgaccaaaccgacgaaaaaaccaacgctaaaaacaacgaaaaaagatccgaaaccgcaaaccacgaaatcgaaagaagtacccacgacgaaacccacggaagaaccgaccataaatacgaccaaaacgaatataataactacgctactaacgtccaatacgacgggaaatccggaactaacgagtcaaatggaaacgtttcattcgacttcgtcggaaggtaatccatcgccgtcgcaagtctcgacgacttccgaatatccgtcacaaccgtcgtcgccaccgaatacgccacgtcaatag

SEQ ID NO: 20 - DNA, 897 bp, unidentified
  source 1..897: mol_type = unassigned DNA; note = respiratory syncytial virus
atgtcgaaaaataaagatcaacgtacggcgaaaacgctagaacgtacgtgggatacgctaaatcatctactatttatatcgtcgtgtctatataaactaaatctaaaatcggtagcgcaaataacgctatcgatactagcgataataatatcgacttcgctaataatagcggcgataatatttatagcgtcggcgaatcataaagtaacgccgacgacggcgataatacaagatgcgacttcgcaaataaaaaatacgacgccgacgtatctaacgcaaaatccgcaactaggtatatcgccgtcgaatccgtcggaaataacgtcgcaaataacgacgatactagcgtcgacgacgccgggtgtaaaatcgacgctacaatcgacgacggtaaaaacgaaaaatacgacgacgacgcaaacgcaaccgtcgaaaccgacgacgaaacaacgtcaaaataaaccgccgtcgaaaccgaataatgattttcattttgaagtatttaattttgtaccgtgttcgatatgttcgaataatccgacgtgttgggcgatatgtaaacgtataccgaataaaaaaccgggtaaaaaaacgacgacgaaaccgacgaaaaaaccgacgctaaaaacgacgaaaaaagatccgaaaccgcaaacgacgaaatcgaaagaagtaccgacgacgaaaccgacggaagaaccgacgataaatacgacgaaaacgaatataataacgacgctactaacgtcgaatacgacgggtaatccggaactaacgtcgcaaatggaaacgtttcattcgacttcgtcggaaggtaatccgtcgccgtcgcaagtatcgacgacttcggaatatccgtcgcaaccgtcgtcgccgccgaatacgccgcgtcaatag

SEQ ID NO: 21 - protein, 574 aa, unidentified
  source 1..574: note = respiratory syncytial virus
MELPILKANAITTILAAVTFCFASSQNITEEFYQSTCSAVSKGYLSALRTGWYTSVITIELSNIKENKCNGTDAKVKLMKQELDKYKNAVTELQLLMQSTPAANNRARRELPRFMNYTLNNTKKTNVTLSKKRKRRFLGFLLGVGSAIASGIAVSKVLHLEGEVNKIKSALLSTNKAVVSLSNGVSVLTSRVLDLKNYIDKQLLPIVNKQSCRISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAEKCKVQSNRVFCDTMYSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 22 - DNA, 14777 bp, unidentified
  source 1..14777: mol_type = unassigned DNA; note = respiratory syncytial virus
acgcgaaaaaatgcgtacaacaaacttgcataaaccaaaaaaatggggcaaataagaatttgataagtaccacttaaatttaactcccttgcttagcgatgggttcgaattcgctatcgatgataaaagtacgtctacaaaatctatttgataatgatgaagtagcgctactaaaaataacgtgttatacggataaactaatacatctaacgaatgcgctagcgaaagcggtaatacatacgataaaactaaatggtatagtatttgtacatgtaataacgtcgtcggatatatgtccgaataataatatagtagtaaaatcgaattttacgacgatgccggtactacaaaatggtggttatatatgggaaatgatggaactaacgcattgttcgcaaccgaatggtctactagatgataattgtgaaataaaattttcgaaaaaactatcggattcgacgatgacgaattatatgaatcaactatcggaactactaggttttgatctaaatccgtaaattataattaatatcaactagcaaatcaatgtcactaacaccattagttaatataaaacttaacagaagacaaaaatggggcaaataaatcaattcagccaacccaaccatggatacgacgcataatgataatacgccgcaacgtctaatgataacggatatgcgtccgctatcgctagaaacgataataacgtcgctaacgcgtgatataataacgcataaatttatatatctaataaatcatgaatgtatagtacgtaaactagatgaacgtcaagcgacgtttacgtttctagtaaattatgaaatgaaactactacataaagtaggttcgacgaaatataaaaaatatacggaatataatacgaaatatggtacgtttccgatgccgatatttataaatcatgatggttttctagaatgtataggtataaaaccgacgaaacatacgccgataatatataaatatgatctaaatccgtaaatttcaacacaatattcacacaatctaaaacaacaactctatgcataactatactccatagtccagatggagcctgaaaattatagtaatttaaaattaaggagagatataagatagaagatggggcaaatacaaagatggctcttagcaaagtcaagttgaatgatacactcaacaaagatcaacttctgtcatccagcaaatacaccatccaacggagcacaggagatagtattgatactcctaattatgatgtgcagaaacacatcaataagttatgtggcatgttattaatcacagaagatgctaatcataaattcactgggttaataggtatgttatatgcgatgtctaggttaggaagagaagacaccataaaaatactcagagatgcgggatatcatgtaaaagcaaatggagtagatgtaacaacacatcgtcaagacattaatggaaaagaaatgaaatttgaagtgttaacattggcaagcttaacaactgaaattcaaatcaacattgagatagaatctagaaaatcctacaaaaaaatgctaaaagaaatgggagaggtagctccagaatacaggcatgactctcctgattgtgggatgataatattatgtatagcagcattagtaataactaaattagcagcaggggacagatctggtcttacagccgtgattaggagagctaataatgtcctaaaaaatgaaatgaaacgttacaaaggcttactacccaaggacatagccaacagcttctatgaagtgtttgaaaaacatccccactttatagatgtttttgttcattttggtatagcacaatcttctaccagaggtggcagtagagttgaagggatttttgcaggattgtttatgaatgcctatggtgcagggcaagtgatgttacggtggggagtcttagcaaaatcagttaaaaatattatgttaggacatgctagtgtgcaagcagaaatggaacaagttgttgaggtttatgaatatgcccaaaaattgggtggtgaagcaggattctaccatatattgaacaacccaaaagcatcattattatctttgactcaatttcctcacttctccagtgtagtattaggcaatgctgctggcctaggcataatgggagagtacagaggtacaccgaggaatcaagatctatatgatgcagcaaaggcatatgctgaacaactcaaagaaaatggtgtgattaactacagtgtactagacttgacagcagaagaactagaggctatcaaacatcagcttaatccaaaagataatgatgtagagctttgagttaataaaaaatggggcaaataaatcatcatggaaaagtttgctcctgaattccatggagaagatgcaaacaacagggctactaaattcctagaatcaataaagggcaaattcacatcacccaaagatcccaagaaaaaagatagtatcatatctgtcaactcaatagatatagaagtaaccaaagaaagccctataacatcaaattcaactattatcaacccaacaaatgagacagatgatactgcagggaacaagcccaattatcaaagaaaacctctagtaagtttcaaagaagaccctacaccaagtgataatcccttttctaaactatacaaagaaaccatagaaacatttgataacaatgaagaagaatccagctattcatacgaagaaataaatgatcagacaaacgataatataacagcaagattagataggattgatgaaaaattaagtgaaatactaggaatgcttcacacattagtagtggcaagtgcaggacctacatctgctcgggatggtataagagatgccatgattggtttaagagaagaaatgatagaaaaaatcagaactgaagcattaatgaccaatgacagattagaagctatggcaagactcaggaatgaggaaagtgaaaagatggcaaaagacacatcagatgaagtgtctctcaatccaacatcagagaaattgaacaacctattggaagggaatgatagtgacaatgatctatcacttgaagatttctgattagttaccactcttcacatcaacacacaataccaacagaagaccaacaaactaaccaacccaatcatccaaccaaacatccatccgccaatcagccaaacagccaacaaaacaaccagccaatccaaaactaaccacccggaaaaaatctataatatagttacaaaaaaaggaaagggtggggcaaatatggaaacatacgtgaacaagcttcacgaaggctccacatacacagctgctgttcaatacaatgtcttagaaaaagacgatgaccctgcatcacttacaatatgggtgcccatgttccaatcatctatgccagcagatttacttataaaagaactagctaatgtcaacatactagtgaaacaaatatccacacccaagggaccttcactaagagtcatgataaactcaagaagtgcagtgctagcacaaatgcccagcaaatttaccatatgcgctaatgtgtccttggatgaaagaagcaaactagcatatgatgtaaccacaccctgtgaaatcaaggcatgtagtctaacatgcctaaaatcaaaaaatatgttgactacagttaaagatctcactatgaagacactcaaccctacacatgatattattgctttatgtgaatttgaaaacatagtaacatcaaaaaaagtcataataccaacatacctaagatccatcagtgtcagaaataaagatctgaacacacttgaaaatataacaaccactgaattcaaaaatgctatcacaaatgcaaaaatcatcccttactcaggattactattagtcatcacagtgactgacaacaaaggagcattcaaatacataaagccacaaagtcaattcatagtagatcttggagcttacctagaaaaagaaagtatatattatgttaccacaaattggaagcacacagctacacgatttgcaatcaaacccatggaagattaacctttttcctctacatcagtgtgttaattcatacaaactttctacctacattcttcacttcaccatcacaatcacaaacactctgtggttcaaccaatcaaacaaaacttatctgaagtcccagatcatcccaagtcattgtttatcagatctagtactcaaataagttaataaaaaatatacacatggacgtccatggggcaaatgcaaacatgtcgaaaaataaagatcaacgtacggcgaaaacgctagaacgtacgtgggatacgctaaatcatctactatttatatcgtcgtgtctatataaactaaatctaaaatcggtagcgcaaataacgctatcgatactagcgataataatatcgacttcgctaataatagcggcgataatatttatagcgtcggcgaatcataaagtaacgccgacgacggcgataatacaagatgcgacttcgcaaataaaaaatacgacgccgacgtatctaacgcaaaatccgcaactaggtatatcgccgtcgaatccgtcggaaataacgtcgcaaataacgacgatactagcgtcgacgacgccgggtgtaaaatcgacgctacaatcgacgacggtaaaaacgaaaaatacgacgacgacgcaaacgcaaccgtcgaaaccgacgacgaaacaacgtcaaaataaaccgccgtcgaaaccgaataatgattttcattttgaagtatttaattttgtaccgtgttcgatatgttcgaataatccgacgtgttgggcgatatgtaaacgtataccgaataaaaaaccgggtaaaaaaacgacgacgaaaccgacgaaaaaaccgacgctaaaaacgacgaaaaaagatccgaaaccgcaaacgacgaaatcgaaagaagtaccgacgacgaaaccgacggaagaaccgacgataaatacgacgaaaacgaatataataacgacgctactaacgtcgaatacgacgggtaatccggaactaacgtcgcaaatggaaacgtttcattcgacttcgtcggaaggtaatccgtcgccgtcgcaagtatcgacgacttcggaatatccgtcgcaaccgtcgtcgccgccgaatacgccgcgtcaatagttacttaaaaacatattatcacaaaaggccttgaccaaccgcggagaatcaaaataaactctggggcaaataacaatggagttgccaatcctcaaagcaaatgcaattaccacaatcctcgctgcagtcacattttgctttgcttctagtcaaaacatcactgaagaattttatcaatcaacatgcagtgcagttagcaaaggctatcttagtgctctaagaactggttggtatactagtgttataactatagaattaagtaatatcaagaaaaataagtgtaatggaacagatgctaaggtaaaattgatgaaacaagaattagataaatataaaaatgctgtaacagaattgcagttgctcatgcaaagcacaccagcagcaaacaatcgagccagaagagaactaccaaggtttatgaattatacactcaacaataccaaaaaaaccaatgtaacattaagcaagaaaaggaaaagaagatttcttggttttttgttaggtgttggatctgcaatcgccagtggcattgctgtatctaaggtcctgcacttagaaggagaagtgaacaagatcaaaagtgctctactatccacaaacaaggccgtagtcagcttatcaaatggagttagtgtcttaaccagcagagtgttagacctcaaaaactatatagataaacaattgttacctattgtgaataagcaaagctgcagaatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgtgatagatacaccttgttggaaattacacacatcccctctatgtacaaccaacacaaaagaagggtcaaacatctgtttaacaagaactgacagaggatggtactgtgacaatgcaggatcagtatctttcttcccacaagctgaaaaatgtaaagttcaatcgaatcgagtattttgtgacacaatgtacagtttaacattaccaagtgaagtaaatctctgcaatgttgacatattcaatcccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgtgattatgtatcaaataaaggggtggacactgtgtctgtaggtaacacattatattatgtaaataagcaagaaggcaaaagtctctatgtaaaaggtgaaccaataataaatttctatgacccattagtattcccctctgatgaatttgatgcatcaatatctcaagtcaatgagaagattaaccagagtttagcatttattcgtaaatccgatgaattattacataatgtaaatgctggtaaatcaaccacaaatatcatgataactactataattatagtgattatagtaatattgttatcattaattgctgttggactgctcctatactgtaaggccagaagcacaccagtcacactaagcaaggatcaactgagtggtataaataatattgcatttagtaactgaataaaaatagcacctaatcatgttcttacaatggtttactatctgctcatagacaacccatctatcattggattttcttaaaatctgaacttcatcgaaactcttatctataaaccatctcacttacactatttaagtagattcctagtttatagttatataaaacacaattgaatgccagtcgaccttaccatctgtaaaaatgaaaactggggcaaatatgtcacgaaggaatccttgcaaatttgaaattcgaggtcattgcttaaatggtaagaggtgtcattttagtcataattattttgaatggccaccccatgcactgcttgtaagacaaaactttatgttaaacagaatacttaagtctatggataaaagtatagataccttatcagaaataagtggagctgcagagttggacagaacagaagagtatgctcttggtgtagttggagtgctagagagttatataggatcaataaacaatataactaaacaatcagcatgtgttgccatgagcaaactcctcactgaactcaatagtgatgatatcaaaaagctgagggacaatgaagagctaaattcacccaagataagagtgtacaatactgtcatatcatatattgaaagcaacaggaaaaacaataaacaaactatccatctgttaaaaagattgccagcagacgtattgaagaaaaccatcaaaaacacattggatatccataagagcataaccatcaacaacccaaaagaatcaactgttagtgatacaaatgaccatgccaaaaataatgatactacctgacaaatatccttgtagtataacttccatactaataacaagtagatgtagagttactatgtataatcaaaagaacacactatatttcaatcaaaacaacccaaataaccatatgtactcaccgaatcaaacattcaatgaaatccattggacctctcaagaattgattgacacaattcaaaattttctacaacatctaggtattattgaggatatatatacaatatatatattagtgtcataacactcaattctaacactcaccacatcgttacattattaattcaaacaattcaagttgtgggacaaaatggatcccattattaatggaaattctgctaatgtttatctaaccgatagttatttaaaaggtgttatctctttctcagagtgtaatgctttaggaagttacatattcaatggtccttatctcaaaaatgattataccaacttaattagtagacaaaatccattaatagaacacatgaatctaaagaaactaaatataacacagtccttaatatctaagtatcataaaggtgaaataaaattagaagaacctacttattttcagtcattacttatgacatacaagagtatgacctcgtcagaacagattgctaccactaatttacttaaaaagataataagaagagctatagaaataagtgatgtcaaagtctatgctatattgaataaactagggcttaaagaaaaggacaagattaaatccaacaatggacaagatgaagacaactcagttattacgaccataatcaaagatgatatactttcagctgttaaagataatcaatctcatcttaaagcagacaaaaatcactctacaaaacaaaaagacacaatcaaaacaacactcttgaagaaattgatgtgttcaatgcaacatcctccatcatggttaatacattggtttaacttatacacaaaattaaacaacatattaacacagtatcgatcaaatgaggtaaaaaaccatgggtttacattgatagataatcaaactcttagtggatttcaatttattttgaaccaatatggttgtatagtttatcataaggaactcaaaagaattactgtgacaacctataatcaattcttgacatggaaagatattagccttagtagattaaatgtttgtttaattacatggattagtaactgcttgaacacattaaataaaagcttaggcttaagatgcggattcaataatgttatcttgacacaactattcctttatggagattgtatactaaagctatttcacaatgaggggttctacataataaaagaggtagagggatttattatgtctctaattttaaatataacagaagaagatcaattcagaaaacgattttataatagtatgctcaacaacatcacagatgctgctaataaagctcagaaaaatctgctatcaagagtatgtcatacattattagataagacagtgtccgataatataataaatggcagatggataattctattaagtaagttccttaaattaattaagcttgcaggtgacaataaccttaacaatctgagtgaactatattttttgttcagaatatttggacacccaatggtagatgaaagacaagccatggatgctgttaaaattaattgcaatgagaccaaattttacttgttaagcagtctgagtatgttaagaggtgcctttatatatagaattataaaagggtttgtaaataattacaacagatggcctactttaagaaatgctattgttttacccttaagatggttaacttactataaactaaacacttatccttctttgttggaacttacagaaagagatttgattgtgttatcaggactacgtttctatcgtgagtttcggttgcctaaaaaagtggatcttgaaatgattataaatgataaagctatatcacctcctaaaaatttgatatggactagtttccctagaaattacatgccatcacacatacaaaactatatagaacatgaaaaattaaaattttccgagagtgataaatcaagaagagtattagagtattatttaagagataacaaattcaatgaatgtgatttatacaactgtgtagttaatcaaagttatctcaacaaccctaatcatgtggtatcattgacaggcaaagaaagagaactcagtgtaggtagaatgtttgcaatgcaaccgggaatgttcagacaggttcaaatattggcagagaaaatgatagctgaaaacattttacaattctttcctgaaagtcttacaagatatggtgatctagaactacaaaaaatattagaattgaaagcaggaataagtaacaaatcaaatcgctacaatgataattacaacaattacattagtaagtgctctatcatcacagatctcagcaaattcaatcaagcatttcgatatgaaacgtcatgtatttgtagtgatgtgctggatgaactgcatggtgtacaatctctattttcctggttacatttaactattcctcatgtcacaataatatgcacatataggcatgcacccccctatataggagatcatattgtagatcttaacaatgtagatgaacaaagtggattatatagatatcacatgggtggcatcgaagggtggtgtcaaaaactgtggaccatagaagctatatcactattggatctaatatctctcaaagggaaattctcaattactgctttaattaatggtgacaatcaatcaatagatataagcaaaccaatcagactcatggaaggtcaaactcatgctcaagcagattatttgctagcattaaatagccttaaattactgtataaagagtatgcaggcataggccacaaattaaaaggaactgagacttatatatcacgagatatgcaatttatgagtaaaacaattcaacataacggtgtatattacccagctagtataaagaaagtcctaagagtgggaccgtggataaacactatacttgatgatttcaaagtgagtctagaatctataggtagtttgacacaagaattagaatatagaggtgaaagtctattatgcagtttaatatttagaaatgtatggttatataatcagattgctctacaattaaaaaatcatgcattatgtaacaataaactatatttggacatattaaaggttctgaaacacttaaaaaccttttttaatcttgataatattgatacagcattaacattgtatatgaatttacccatgttatttggtggtggtgatcccaacttgttatatcgaagtttctatagaagaactcctgacttcctcacagaggctatagttcactctgtgttcatacttagttattatacaaaccatgacttaaaagataaacttcaagatctgtcagatgatagattgaataagttcttaacatgcataatcacgtttgacaaaaaccctaatgctgaattcgtaacattgatgagagatcctcaagctttagggtctgagagacaagctaaaattactagcgaaatcaatagactggcagttacagaggttttgagtacagctccaaacaaaatattctccaaaagtgcacaacattatactactacagagatagatctaaatgatattatgcaaaatatagaacctacatatcctcatgggctaagagttgtttatgaaagtttacccttttataaagcagagaaaatagtaaatcttatatcaggtacaaaatctataactaacatactggaaaaaacttctgccatagacttaacagatattgatagagccactgagatgatgaggaaaaacataactttgcttataaggatacttccattggattgtaacagagataaaagagagatattgagtatggaaaacctaagtattactgaattaagcaaatatgttagggaaagatcttggtctttatccaatatagttggtgttacatcacccagtatcatgtatacaatggacatcaaatatactacaagcactatatctagtggcataattatagagaaatataatgttaacagtttaacacgtggtgagagaggacccactaaaccatgggttggttcatctacacaagagaaaaaaacaatgccagtttataatagacaagtcttaaccaaaaaacagagagatcaaatagatctattagcaaaattggattgggtgtatgcatctatagataacaaggatgaattcatggaagaactcagcataggaacccttgggttaacatatgaaaaggccaagaaattatttccacaatatttaagtgtcaattatttgcatcgccttacagtcagtagtagaccatgtgaattccctgcatcaataccagcttatagaacaacaaattatcactttgacactagccctattaatcgcatattaacagaaaagtatggtgatgaagatattgacatagtattccaaaactgtataagctttggccttagtttaatgtcagtagtagaacaatttactaatgtatgtcctaacagaattattctcatacctaagcttaatgagatacatttgatgaaacctcccatattcacaggtgatgttgatattcacaagttaaaacaagtgatacaaaaacagcatatgtttttaccagacaaaataagtttgactcaatatgtggaattattcttaagtaataaaacactcaaatctggatctcatgttaattctaatttaatattggcacataaaatatctgactattttcataatacttacattttaagtactaatttagctggacattggattctgattatacaacttatgaaagattctaaaggtatttttgaaaaagattggggagagggatatataactgatcatatgtttattaatttgaaagttttcttcaatgcttataagacctatctcttgtgttttcataaaggttatggcaaagcaaagctggagtgtgatatgaacacttcagatcttctatgtgtattggaattaatagacagtagttattggaagtctatgtctaaggtatttttagaacaaaaagttatcaaatacattcttagccaagatgcaagtttacatagagtaaaaggatgtcatagcttcaaattatggtttcttaaacgtcttaatgtagcagaattcacagtttgcccttgggttgttaacatagattatcatccaacacatatgaaagcaatattaacttatatagatcttgttagaatgggattgataaatatagatagaatacacattaaaaataaacacaaattcaatgatgaattttatacttctaatctcttctacattaattataacttctcagataatactcatctattaactaaacatataaggattgctaattctgaattagaaaataattacaacaaattatatcatcctacaccagaaaccctagagaatatactagccaatccgattaaaagtaatgacaaaaagacactgaatgactattgtataggtaaaaatgttgactcaataatgttaccattgttatctaataagaagcttattaaatcgtctgcaatgattagaaccaattacagcaaacaagatttgtataatttattccctatggttgtgattgatagaattatagatcattcaggcaatacagccaaatccaaccaactttacactactacttcccaccaaatatctttagtgcacaatagcacatcactttactgcatgcttccttggcatcatattaatagattcaattttgtatttagttctacaggttgtaaaattagtatagagtatattttaaaagatcttaaaattaaagatcccaattgtatagcattcataggtgaaggagcagggaatttattattgcgtacagtagtggaacttcatcctgacataagatatatttacagaagtctgaaagattgcaatgatcatagtttacctattgagtttttaaggctgtacaatggacatatcaacattgattatggtgaaaatttgaccattcctgctacagatgcaaccaacaacattcattggtcttatttacatataaagtttgctgaacctatcagtctttttgtctgtgatgccgaattgtctgtaacagtcaactggagtaaaattataatagaatggagcaagcatgtaagaaagtgcaagtactgttcctcagttaataaatgtatgttaatagtaaaatatcatgctcaagatgatattgatttcaaattagacaatataactatattaaaaacttatgtatgcttaggcagtaagttaaagggatcggaggtttacttagtccttacaataggtcctgcgaatatattcccagtatttaatgtagtacaaaatgctaaattgatactatcaagaaccaaaaatttcatcatgcctaagaaagctgataaagagtctattgatgcaaatattaaaagtttgataccctttctttgttaccctataacaaaaaaaggaattaatactgcattgtcaaaactaaagagtgttgttagtggagatatactatcatattctatagctggacgtaatgaagttttcagcaataaacttataaatcataagcatatgaacatcttaaaatggttcaatcatgttttaaatttcagatcaacagaactaaactataaccatttatatatggtagaatctacatatccttacctaagtgaattgttaaacagcttgacaaccaatgaacttaaaaaactgattaaaatcacaggtagtctgttatacaactttcataatgaataatgaataaagatcttataataaaaattcccatagctatacactaacactgtattcaattatagttattaaaaattaaaaatcgtacgattttttaaataacttttagtgaactaatcctaaagttatcattttaatcttggaggaataaatttaaaccctaatctaattggtttatatgtgtattaactaaattacgagatattagtttttgacactttttttctcgt

SEQ ID NO: 23 - protein, 139 aa, unidentified; identical to SEQ ID NO: 5
  VARIANT 3: note = S or C
  VARIANT 5: note = S or T
  VARIANT 8: note = M or V
  VARIANT 11: note = V or I
  VARIANT 17: note = F or L
  VARIANT 18: note = D or N
  VARIANT 31: note = T or A
  VARIANT 36: note = H, L, or Q
  VARIANT 45: note = V or T
  VARIANT 57: note = V or I
  VARIANT 64: note = D or E
  VARIANT 65: note = I, A, or V
  VARIANT 68: note = N or D
  VARIANT 70: note = N or S
  VARIANT 82: note = V, I, or A
  VARIANT 84: note = Q or R
  VARIANT 90: note = W or any amino acid
  VARIANT 92: note = M or L
  VARIANT 93: note = M or I
  VARIANT 101: note = P or L
  VARIANT 104: note = L or V
  VARIANT 105: note = L, M, or I
  VARIANT 106: note = D or V
  VARIANT 115: note = K or R
  VARIANT 116: note = K or R
  VARIANT 118: note = S or any amino acid
  VARIANT 121: note = T or V
  VARIANT 124: note = N or D
  VARIANT 126: note = M or I
  VARIANT 127: note = N or S
  VARIANT 129: note = L or I
  VARIANT 131: note = E or D
  VARIANT 135: note = F or L
  VARIANT 138: note = N or H
  VARIANT 139: note = P or S or deleted
  source 1..139: note = respiratory syncytial virus

SEQ ID NO: 24 - protein, 124 aa, unidentified; identical to SEQ ID NO: 8
  VARIANT 2: note = D or S
  VARIANT 4: note = T, A, or K
  VARIANT 5: note = H, S, or N
  VARIANT 6: note = N or P
  VARIANT 7: note = D, G, or E
  VARIANT 8: note = T or N
  VARIANT 10: note = P, M, Q, S, or A
  VARIANT 12: note = R or G
  VARIANT 14: note = M or I
  VARIANT 21: note = L or M
  VARIANT 23: note = L, M, or I
  VARIANT 24: note = I, D, or E
  VARIANT 25: note = T or S
  VARIANT 26: note = I or V
  VARIANT 28: note = I or T
  VARIANT 32: note = R or K
  VARIANT 33: note = D or E
  VARIANT 38: note = R or K
  VARIANT 45: note = H or N
  VARIANT 50: note = R or K
  VARIANT 55: note = R or K
  VARIANT 59: note = F or L
  VARIANT 60: note = T or A
  VARIANT 68: note = K or N
  VARIANT 72: note = K or R
  VARIANT 76: note = T or A
  VARIANT 77: note = K or I
  VARIANT 81: note = T or S
  VARIANT 98: note = N or any amino acid
  VARIANT 100: note = D or G
  VARIANT 103: note = L or I
  VARIANT 108: note = I or V
  VARIANT 118: note = Y or H
  source 1..124: note = respiratory syncytial virus

SEQ ID NO: 25 - protein, 6 aa, synthetic construct
GGGGGG

SEQ ID NO: 26 - protein, 5 aa, synthetic construct
GGGGT

SEQ ID NO: 27 - protein, 6 aa, synthetic construct
GGGPPP

SEQ ID NO: 28 - protein, 7 aa, synthetic construct
GGGAPPP

SEQ ID NO: 29 - protein, 574 aa, unidentified
  source 1..574: note = respiratory syncytial virus
MELPILKANAITTILAAVTFCFASSQNITEEFYQSTCSAVSKGYLSALRTGWYTSVITIELSNIKKNKCNGTDAKVKLMKQELDKYKNAVTELQLLMQSTPAANNRARRELPRFMNYTLNNTKKTNITLSKKRKRRFLGFLLGVGSAIASGIAVSKVLHLEGEVNKIKSALLSTNKAVVSLSNGVSVLTSRVLDLKNYIDKQLLPIVNKQSCRISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAEKCKVQSNRVFCDTMYSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 30 - protein, 574 aa, unidentified
  source 1..574: note = respiratory syncytial virus
MELPILKANAITTILAAVTFCFASSQNITEEFYQSTCSAVSKGYLSALRTGWYTSVITIELSNIKENKCNGTDAKVKLMKQELDKYKNAVTELQLLMQSTPAANNRARRELPRFMNYTLNNTKKTNITLSKKRKRRFLGFLLGVGSAIASGIAVSKVLHLEGEVNKIKSALLSTNKAVVSLSNGVSVLTSKVLDLKNYIDKQLLPIVNKQSCRISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 31 - protein, 574 aa, unidentified
  source 1..574: note = respiratory syncytial virus
MELPILKANAITTILAAVTFCFASSQNITEEFYQSTCSAVSKGYLSALRTGWYTSVITIELSNIKENKCNGTDAKVKLMKQELDKYKNAVTELQLLMQSTPAANNRARRELPRFMNYTLNNTKKTNVTLSKKRKRRFLGFLLGVGSAIASGIAVSKVLHLEGEVNKIKSALLSTNKAVVSLSNGVSVLTSKVLDLKNYIDKQLLPIVNKQSCRISNIETVIEFQQKNNRLLEITREFSVNVGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

(19) United States
(12) Patent Application Publication
(10) Pub. No.: US 2024/0082385 A1
(43) Pub. Date: Mar. 14, 2024

(54) RSV VACCINES AND METHODS OF ADMINISTERING SAME

(71) Applicant: Meissa Vaccines, Inc., Redwood City, CA (US)

(72) Inventors: Martin Moore, San Carlos, CA (US); Roderick Tang, San Mateo, CA (US); Oliver Medzihradsky, San Francisco, CA (US); Fiona Cameron, Grass Valley, CA (US)

(21) Appl. No.: 18/202,836

(22) Filed: May 26, 2023

Related U.S. Application Data
(60) Provisional application No. 63/346,623, filed on May 27, 2022.
(60) Provisional application No. 63/382,527, filed on Nov. 6, 2022.

(51) Int. Cl. A61K 39/155

(52) U.S. Cl. CPC A61K 39/155; A61K 2039/5254

(57) ABSTRACT

The invention relates generally to pediatric dosing regimens for RSV vaccines.

### CROSS-REFERENCE TO RELATED APPLICATIONS

This application claims the benefit of, and priority to, U.S. Provisional Application No. 63/346,623, filed May 27, 2022 and U.S. Provisional Application No. 63/382,527, filed Nov. 6, 2022, the contents of each of which are hereby incorporated by reference in their entirety.

### REFERENCE TO A SEQUENCE LISTING XML

This application contains a Sequence Listing which has been submitted electronically in XML format. The Sequence Listing XML is incorporated herein by reference. Said XML file, created on Oct. 12, 2023, is named MSA-008US_SL.xml and is 159,710 bytes in size.

### FIELD OF THE INVENTION

The invention relates generally to pediatric dosing regimens for RSV vaccines.

### BACKGROUND

Respiratory syncytial virus (RSV) leads to lower respiratory tract infections. Immunocompromised patients, premature infants, and children are particularly at risk to severe disease. RSV is the leading cause of viral death in infants. RSV treatments are focused on prevention from infection and improving respiration. Palivizumab is a humanized monoclonal antibody that can be given prophylactically. Palivizumab is not effective after RSV infection, and protection ends shortly after treatment stops. Attenuated RSV vaccine candidates have failed because of suboptimal immunogenicity in infants and suboptimal stability that leads to genetic reversion towards undesirable wild-type sequences. See Teng, Infectious Disorders—Drug Targets, 2012, 12(2): 129-3. Thus, there is a need to find an attenuated RSV vaccine that is appropriately immunogenic, sufficiently stable, and safe for use in infants and children, as well as safe dosing regimens for administering such an attenuated RSV vaccine.

### SUMMARY

The disclosure relates, in part, to a method of administering a live attenuated respiratory syncytial virus (RSV) vaccine to a subject in need thereof. The method can include intranasally administering the live attenuated RSV vaccine in an amount of from about 10³ pfu/dose to about 10⁶ pfu per dose to the subject, wherein the subject is a child between 6 and 59 months of age (e.g., between about 6 and 24 months of age, between about 6 and 36 months of age, or between about 15 and 59 months of age), wherein administration of the live attenuated RSV vaccine results in at least one of (a) an increase in RSV-specific serum neutralizing antibody (nAb) titers in the subject, (b) an increase in serum binding (RSV F-specific) IgG concentrations in the subject, and (c) an increase in nasal mucosal binding (RSV F-specific) IgA concentrations in the subject, and wherein the live attenuated RSV vaccine comprises a genome comprising one or more of SEQ ID NOs: 6, 9, and 20.

In certain embodiments, the administration results in a reduced likelihood or severity of infection by RSV.

In certain embodiments, the administration is in a single dose of about 10³ pfu/dose. In certain embodiments, the administration is in a single dose of about 10⁴ pfu/dose. In certain embodiments, the administration is in a single dose of about 10⁵ pfu/dose. In certain embodiments, the administration is in a single dose of about 10⁶ pfu/dose. In certain embodiments, the administration is in a single dose of about 10⁷ pfu/dose.

In certain embodiments, the administration is in two doses of about 10³ pfu/dose. In certain embodiments, the administration is in two doses of about 10⁴ pfu/dose. In certain embodiments, the administration is in two doses of about 10⁵ pfu/dose. In certain embodiments, the administration is in two doses of about 10⁶ pfu/dose. In certain embodiments, the administration is in two doses of about 10⁷ pfu/dose. In certain embodiments, the two doses are administered between about 3 and about 6 weeks apart. In certain embodiments, the two doses are administered between about 4 and about 5 weeks apart. In certain embodiments, the two doses are administered between about 28, 29, or 30 days apart. In certain embodiments, administration of the live attenuated RSV vaccine results in an increase in RSV-specific serum neutralizing antibody (nAb) titers in the subject.

In certain embodiments, administration of the live attenuated RSV vaccine results in an increase in serum binding (RSV F-specific) IgG concentrations in the subject. In certain embodiments, administration of the live attenuated RSV vaccine results in an increase in nasal mucosal binding (RSV F-specific) IgA concentrations in the subject.

In certain embodiments, the live attenuated RSV vaccine comprises SEQ ID NOs: 6, 9, and 20. In certain embodiments, the live attenuated RSV vaccine comprises SEQ ID NO: 22.

In certain embodiments, the intranasal administration is by drops or by nasal atomizer.

In certain embodiments, the disclosure relates to the polynucleotide sequences of respiratory syncytial virus (RSV). In certain embodiments, the disclosure relates to isolated or recombinant nucleic acids and polypeptides comprising desirable nucleic acid sequences and mutations disclosed herein. In certain embodiments, isolated or recombinant RSV comprising the nucleic acids and polypeptides disclosed herein (e.g., attenuated recombinant RSV) are also provided, as are immunogenic compositions including such nucleic acids, polypeptides, and RSV genomes that are suitable for use as vaccines.

Attenuated or killed RSV containing these nucleic acids and mutation in the form of copied nucleic acids (e.g., cDNAs) are also contemplated. In certain embodiments, this disclosure relates to isolated nucleic acids, recombinant respiratory syncytial virus (RSV) with codon deoptimization, vaccines produced therefrom, and vaccination methods related thereto. In certain embodiments, the recombinant RSV comprises the genes NS1, NS2, N, P, M, SH, G, F, M2, and L of strain A2, line 19, or Long strain or variants thereof. In certain embodiments, the codon deoptimization is in the nonstructural genes NS1 and NS2 and optionally in a gene G and optionally in a gene L. In further embodiments, the gene SH is deleted. In further embodiments, the gene F is mutated, e.g., an I to V mutation corresponding to residue 557 of RSV strain line 19 F protein.

In certain embodiments, the disclosure relates to isolated nucleic acids encoding deoptimized genes NS1 and/or NS2 and optionally the gene G and optionally the gene L of a wild-type human RSV or variant wherein the nucleotides are substituted such that a codon to produce Gly is GGT, a codon to produce Asp is GAT, a codon to produce Glu is GAA, a codon to produce His is CAT, a codon to produce Ile is ATA, a codon to produce Lys is AAA, a codon to produce Leu is CTA, a codon to produce Asn is AAT, a codon to produce Gin is CAA, a codon to produce Val is GTA, or a codon to produce Tyr is TAT, or combinations thereof. In certain embodiments, a gene in the isolated nucleic acid further comprises a combination of at least two, three, four, five, six, seven, eight nine, ten, or all of the individual codons. In certain embodiment, a gene in the isolated nucleic acid comprises at least 20, 30, 40, or 50 or more of the codons.

In certain embodiment, this disclosure relates to isolated nucleic acid as disclosed herein wherein the nucleotides are substituted such that a codon to produce Ala is GCG, a codon to produce Cys is TGT, a codon to produce Phe is TTT, a codon to produce Pro is CCG, a codon to produce Arg is CGT, a codon to produce Ser is TCG, or a codon to produce Thr is ACG, or combinations thereof. In certain embodiments, a gene containing the nucleic acid comprises a combination of at least two, three, four, five, six, seven, eight nine, ten, eleven, twelve, thirteen, fourteen, fifteen, sixteen, or all of the individual codons. In certain embodiments, a gene in the isolated nucleic acid further comprises at least 20, 30, 40, or 50 or more of the codons.

In certain embodiments, the disclosure relates to nucleic acids disclosed herein encoding an NS1 having SEQ ID NO: 5 and SEQ ID NO: 23:

MGX¹NX²LSX³IKX⁴RLQNLX⁵X⁶NDEVALLKITCYX⁷DKLIX⁸LTNALA
KAX⁹IHTIKLNGIVFX¹⁰HVITSSX¹¹X¹²CPX¹³NX¹⁴IVVKSNFTTMP
X¹⁵LX¹⁶NGGYIX¹⁷EX¹⁸X¹⁹ELTHSQX²⁰NGX²¹X²²X²³DNCEIKFS
X²⁴X²⁵LX²⁶DSX²⁷MTX²⁸YX²⁹X³⁰QX³¹SX³²LLGX³³DLX³⁴X³⁵,

wherein
X¹-X³⁵ are any amino acid or
X¹ is S or C; X² is S or T; X³ is M or V; X⁴ is V or I; X⁵ is F or L; X⁶ is D or N; X⁷ is T or A; X⁸ is H, L, or Q; X⁹ is V or T; X¹⁰ is V or I; X¹¹ is D or E; X¹² is I, A, or V; X¹³ is N or D; X¹⁴ is N or S; X¹⁵ is V, I, or A; X¹⁶ is Q or R; X¹⁷ is W or any amino acid; X¹⁸ is M or L; X¹⁹ is M or I; X²⁰ is P or L; X²¹ is L or V; X²² is L, M, or I; X²³ is D or V; X²⁴ is K or R; X²⁵ is K or R; X²⁶ is S or any amino acid; X²⁷ is T or V; X²⁸ is N or D; X²⁹ is M or I; X³⁰ is N or S; X³¹ is L or I; X³² is E or D; X³³ is F or L; X³⁴ is Nor H; and X³⁵ is P or S or deleted.

In certain embodiments, the disclosure relates to nucleic acids disclosed herein encoding an NS1 of RSV as provided in NCBI Accession number NP_044589.1, NP_056856.1, P04544.1, AEQ63513.1, AFM55237.1, AFV32554.1, Q86306.1, AFV32528.1, AFM55248.1, AFM95358.1, AFV32568.1, ACY68428.1, CBW45413.1, ACO83290.1, AFM55347.1, CBW45433.1, AEQ63459.1, AFM55204.1, AFV32572.1, AFV32558.1, CBW45429.1, CBW45445.1, AFV32596.1, CBW45481.1, CBW47561.1, P24568.1, AAR14259.1, CBW45451.1, CBW45447.1, CBW45471.1, BAE96914.1, CBW45463.1, CBW45473.1, or CBW45467.1 or variants comprising one, two, or three amino acid insertions, deletions, substitutions, or conserved substitutions.

In certain embodiments, the disclosure relates to an isolated nucleic acid comprising SEQ ID NO: 6 or SEQ ID NO: 7 or a sequence with 60%, 70%, 80%, 90%, 95% or greater sequence identity thereto.

In certain embodiments, the disclosure relates to nucleic acids disclosed herein encoding an NS2 having SEQ ID NO: 8 and SEQ ID NO: 24:

MX¹TX²X³X⁴X⁵X⁶TX⁷QX⁸LX⁹ITDMRPX¹⁰SX¹¹X¹²X¹³X¹⁴1X¹⁵
SLTX¹⁶X¹⁷IITHX¹⁸FIYLINX¹⁹ECIVX²⁰KLDEX²¹QATX²²X²³
FLVNYEMX²⁴LLHX²⁵VGSX²⁶X²⁷YKKX²⁸TEYNTKYGTFPMPIFI
X²⁹HX³⁰GFX³¹ECIGX³²KPTKHTPIIX³³KYDLNP,

wherein X¹-X³³ are any amino acid or X¹ is D or S; X² is T, A, or K; X³ is H, S, or N; X⁴ is N or P; X⁵ is D, G, or E; X⁶ is T or N; X⁷ is P, M, Q, S, or A; X⁸ is R or G; X⁹ is M or I; X¹⁰ is L or M; X¹¹ is L, M, or; X¹² is I, D, or E; X¹³ is T or S; X¹⁴ is I or V; X¹⁵ is I or T; X¹⁶ is R or K; R¹⁷ is D or E; R¹⁸ is R or K; R¹⁹ is H or N; X²⁰ is R or K; X²¹ is R or K; X²² is F or L; X²³ is T or A; X²⁴ is K or N; X²⁵ is K or R; X²⁶ is T or A; X²⁷ is K or I; X²⁸ is T or S; X²⁹ is N or any amino acid; X³⁰ is D or G; X³¹ is L or I; X³² is I or V; and X³³ is Y or H.

In certain embodiments, the disclosure relates to nucleic acids disclosed herein encoding an NS1 having an NS2 of RSV as provided in NCBI Accession number

NP_044590.1, NP_056857.1, CBW45420.1, AFM95337.1, CBW45416.1, CBW45430.1, AFV32529.1, Q86305.1, AEQ63383.1, CBW45424.1, AFM55546.1, CBW45444.1, P04543.2, AFM55326.1, AFM55425.1, AFM55381.1, AFM55458.1, AFM55216.1, AAB59851.1, AEQ63372.1, AFM55337.1, CBW45426.1, AFV32515.1, AFV32519.1, AAR14260.1, CBW47562.1, AFV32643.1, P24569.1, AFV32657.1 AFI25256.1, CBW45480.1, AFV32605.1, AEQ63580.1, AFV32627.1, AFV32665.1, CBW45482.1, CBW45478.1, CBW45462.1, AEQ63635.1, CBW45448.1, CBW45464.1, CBW45484.1, or CBW45474.1 or variants comprising one, two or three amino acid insertions, deletions, substitutions, or conserved substitutions.

In certain embodiments, the disclosure relates to an isolated nucleic acid comprising SEQ ID NO: 9 or SEQ ID NO: 10 or a sequence with 60%, 70%, 80%, 90%, 95% or greater sequence identity thereto.

In certain embodiments, the disclosure relates to recombinant vectors comprising a nucleic acid disclosed herein.

In certain embodiments, the disclosure relates to an attenuated recombinant RSV comprising a nucleic acid disclosed herein.

In certain embodiments, the disclosure relates to expression system comprising a vector disclosed herein or an attenuated recombinant RSV disclosed herein.

In certain embodiments, the disclosure relates to vaccines comprising an attenuated recombinant RSV disclosed herein.

In certain embodiments, the disclosure relates to methods of vaccination comprising administering an effective amount of a vaccine disclosed herein to a subject at risk of an RSV infection.

In certain embodiments, the subject is younger than 2 months or 6 months of age, under 1 year of age, born prematurely, have congenital heart or lung disease, having chemotherapy or a transplantation, or diagnosed with asthma, congestive heart failure or chronic obstructive pulmonary disease, leukemia, or HIV/AIDS.

In certain embodiments, vaccine is administered in combination with motavizumab, palivizumab, or another humanized monoclonal antibody directed against an epitope in the antigenic site II of the F protein of RSV.

In certain embodiments, the disclosure relates to vectors disclosed herein comprising a bacterial artificial chromosome (BAC), and a nucleic acid sequence comprising respiratory syncytial virus (RSV), and the BAC contains all genes that are essential for the generation of an infectious viral particle in a host cell. The nucleic acid sequence may be a viral genome or antigenome in operable combination with a regulatory element. Typically, the bacterial artificial chromosome comprises one or more genes selected from the group consisting of oriS, repE, parA, and parB genes of factor F in operable combination with a selectable marker, e.g., a gene that provides resistance to an antibiotic.

The nucleic acid sequence may be the genomic or antigenomic sequence of the virus which is optionally mutated as provided herein, e.g., RSV strain which is optionally mutated. In certain embodiments, the expression vector is a plasmid comprising MluI, ClaI, BstBl, SacI restriction endonuclease cleavage sites and optionally an AvrII restriction endonuclease cleavage site outside the region of the wild-type viral sequence or outside the sequences that encode viral genes or outside the viral genome or antigenome. In certain embodiments, the nucleic acid sequence further comprises a selectable marker or reporter gene in operable combination therewith, e.g., a gene that encodes a fluorescent protein.

In certain embodiments, the disclosure relates to isolated bacteria comprising one or more vectors disclosed herein, and other embodiments, the disclosure relates to an isolated cell comprising one or more vectors disclosed herein. In certain embodiments, the vector comprises an RSV antigenome and one or more vectors selected from the group consisting of: a vector encoding an N protein of RSV, a vector encoding a P protein of RSV, a vector encoding an L protein of RSV, and a vector encoding an M2-1 protein of RSV. Typically, the vector comprises a regulatory element, e.g., promoter, and the isolated eukaryotic cell expresses a nucleic acid or polypeptide that activates the regulatory element, e.g., encodes a polypeptide that activates transcription downstream of the promoter. In certain embodiments, the promoter is T7, and the polypeptide that activates transcription downstream of the promoter is T7 RNA polymerase.

In certain embodiments, the disclosure relates to methods of generating respiratory syncytial virus (RSV) particles comprising inserting a vector with a BAC gene and a RSV antigenome into an isolated eukaryotic cell and inserting one or more vectors selected from the group consisting of: a vector encoding an N protein of RSV, a vector encoding a P protein of RSV, a vector encoding an L protein of RSV, and a vector encoding an M2-1 protein of RSV into the cell under conditions such that RSV virion is formed. Inserting a vector into a cell may occur by physically injecting, electroporating, or mixing the cell and the vector under conditions such that the vector enters the cell.

In certain embodiments, the disclosure relates to the stability of the line 19 F557 mutant virus compared to other strains, and Val at 557 making RSV expressing line 19 F even more thermostable. Val at position 557 in other strains is also likely stabilizing; thus the 557 position is important for thermal stability. In certain embodiments, the disclosure contemplates other mutations in line 19 F or other RSV strains at position 557 (any amino acid, e.g., alanine, valine, isoleucine, leucine) in any F strain context, that improves thermostability of the RSV virus.

In certain embodiments, the disclosure contemplates RSV F polypeptide comprising an alanine, valine, or leucine at position 557, e.g., alanine or leucine in position 557 of SEQ ID NO: 17.

In certain embodiments, the disclosure relates to certain desirable sequence of RSV F polypeptides e.g., line 19 sequences comprising a valine at position 557, e.g., SEQ ID NO: 17, and recombinant nucleic acids encoding the same. In certain embodiments, the disclosure contemplates recombinant vectors comprising nucleic acids encoding these polypeptides and cells comprising said vectors.

In certain embodiments, the disclosure relates to immunogenic compositions comprising an immunologically effective amount of a recombinant respiratory syncytial virus (RSV), RSV polypeptide, RSV particle, RSV virus-like particle, and/or nucleic acid disclosed herein. In certain embodiments, the disclosure relates to methods for stimulating the immune system of an individual to produce a protective immune response against RSV. In certain embodiments, an immunologically effective amount of a RSV, polypeptide, and/or nucleic acid disclosed herein is administered to the individual in a physiologically acceptable carrier.

In certain embodiments, the disclosure relates to medicaments and vaccine products comprising nucleic acids disclosed herein for uses disclosed herein.

In certain embodiments, the disclosure relates to uses of nucleic acids or vectors disclosed herein for the manufacture of a medicament for uses disclosed herein.

### BRIEF DESCRIPTIONS OF THE DRAWINGS

FIG. 1 shows a table with the least used codons in human genes and in specific RSV strains.

FIG. 2 shows growth data for kRSV-dNS1h in BEAS-2B (top) and Vero cell lines (bottom). Growth curves of kRSV-A2 (open circle) and kRSV-dNSh (closed circle) in HEp-2 (A), Vero (B) and BEAS-2B (C) at 37° C. infected at MOI of 0.01, as well as in differentiated NHBE/ALI cells infected at MOI of 0.2 (D) or 2.0 (E).

FIG. 3 shows data on viral load experiments using certain embodiments disclosed herein. Time course images for NHBE cells infection at MOI of 0.2, showing mKate2 fluorescence produced by the recombinant viruses. *P<0.05

FIG. 4 shows a gel after insertion of galK operon into BAC-RSV by recombineering. MluI digest. Lane 1, ladder marker. Mini-prep BAC DNAs (lanes 2 to 7). Lane 8, parental BAC-RSV “C2” clone. Lane 9, galK-containing plasmid. galK operon has a Mlu I restriction site that serves as a marker for introduction of galK by homologous recombination.

FIG. 5 shows a gel after deletion of galK operon from BAC-RSV by recombineering. MluI digest of galK-containing plasmid (lane 2), BAC mini-prep DNAs (lanes 3-7), and parental BAC-RSV clone C2 (lane 8).

FIGS. 6A-E schematically illustrates steps for creating a BAC-RSV. Three plasmids with RSV segments are generated (see experimental); A) pKBS3 is cut at BstBl and MluI sites to linearize, and is ligated to an oligonucleotide adapter providing pKBS5; B) pSynRSV #2 with Sacl and Clal is cut and ligated to pKBS5 providing pKBS5-2; C) pSynRSV #3 with AvrII and MluI is cut and ligated to pKBS5_2 providing pKBS5_23; D) pSynRSV #1 with BstBl and Sacl is cut and ligated to pKBS5_2_3 providing pKBS5_1_2_3. E). Recombineering is used to delete nucleotides between two Clal sites generating pSynRSV-line 19F.

FIG. 7A shows data showing indicating the immunogenicity of an RSV strain with an F gene 1557 to V mutation. Mice were infected with indicated doses of A2-K-line19F, A2-line19F-I557 V, or A2-K-A2GF and 29 days later challenged with RSV strain 12-35. FIG. 7B shows lung viral load as was measured day 4 post-challenge. The dotted line indicates the limit of detection.

FIG. 8 shows data indicating the superior thermostability of RSV strains with an A2-line 19 F gene 1557 to V mutation (SEQ ID NO: 17). Viruses were incubated at indicated temperatures and viral titers were measured every day for 6 days. The results at 4° C. are statistically significant between viruses (P<0.01). The results at 37° C. demonstrate the same phenotype.

FIG. 9 illustrates an RSV sequence comparison of strain 19, 1557 V mutation (SEQ ID NO: 17) (Query) and the typical RSV strain 19 sequence (Sbjct) (SEQ ID NO: 29).

FIG. 10 illustrates an RSV sequence comparison of strain 19, 1557 V mutation (SEQ ID NO: 17) (Query) and sequence 61 from U.S. Pat. No. 7,951,384 (Sbjct) (SEQ ID NO: 30).

FIG. 11 illustrates an RSV sequence comparison of strain 19, 1557 V mutation (SEQ ID NO: 17) (Query) sequence 12 from U.S. Pat. No. 8,580,270 (Sbjct) (SEQ ID NO: 31).

FIGS. 12A-C show data on attenuation, efficacy, and immunogenicity of embodiments disclosed herein. (FIG. 12A) 6-8 week old BALB/c mice (n=5 per group) were infected i.n. with 1.6×10⁵ FFU of kRSV-A2 (open circle) or kRSV-dNSh (closed circle) and lung viral titer was assayed on days 1, 2, 4, 6, and 8 p.i. Data represent one of two replicate experiments with similar results. *P<0.05. (FIG. 12B) BALB/c mice were vaccinated i.n. with varying doses (10⁵ FFU, 10⁴ FFU, and 10³ FFU) of kRSV-A2 (open circle) or kRSV-dNSh (closed circle), or mock-infected, and 100 days after vaccination, mice were challenged with 1.6×10⁶ PFU RSV 12-35 strain. Lung peak viral loads were measured on day 4 after challenge. Each symbol represents one mouse. Dashed lines (FIGS. 12A and 12B) denote the limit of detection for plaque assay. Titers below the limit of detection were assigned half the value of the limit of detection. (FIG. 12C) BALB/c mice (n=5 per group) were mock-infected or infected with 10⁵ FFU of either kRSV-A2 or kRSV-dNSh and serum nAb titers were measured at indicated days after infection. *P<0.05.

FIGS. 13A and 13B show data on vaccine efficacy for certain embodiments disclosed herein. 6-8 week old BALB/c mice (n=5 per group) were mock-infected or vaccinated with varying indicated doses of kRSV-A2 (open circle) or kRSV-dNSh (closed circle). Mice were challenged 28 days later with (FIG. 13A) 2×10⁶ PFU RSV A2-line 19 strain or (FIG. 13B) 5X 10⁵ PFU RSV 12-35. Lung viral loads were measured day 4 after challenge. Each symbol represents one mouse. Dashed lines denote the limit of detection for plaque assay. Titers below the limit of detection were assigned half the value of the limit of detection.

FIGS. 14A-C show data on the expression of NS1 and NS2 proteins during RSV infection in cell lines. HEp-2 (FIG. 14A), BEAS-2B (FIG. 14B) and Vero (FIG. 14C) cells were mock-infected or infected with either kRSV-A2, kRSV-dNSh, or kRSV-dNSv at MOI 5. Twenty hr p. i., NS1 and NS2 protein levels were analyzed by western blot and densitometry. Representative blots are shown on the left. Densitometry from 2-3 independent experiments is shown on the right. After normalizing to RSV N protein levels, NS1 and NS2 protein levels expressed by each virus were normalized to those during kRSV-A2 infection and expressed as percentage ±SEM. Unfilled bars represent kRSV-A2, gray bars represent kRSV-dNSv, and black bars represent kRSV-dNSh.

FIG. 15 shows the incidence of adverse events (AEs) occurring within 14 days of vaccinations of subjects (infants) aged 6-24 months according to dosage regimen.

FIG. 16 shows neutralizing antibody (nAb) response in subjects (infants) vaccinated with two 10⁶ PFU doses given 29 days apart. As shown, 78% of infants showed a greater than 4-fold rise in serum nAb, with a mean fold rise in nAb of 64-fold, a median fold rise in nAb of 10-fold, and mean geometric mean titer (GMT) of 798 and a median GMT of 43.

FIG. 17 shows the serum nAb, serum pre-F IgG, and nasal pre-F IgA measured in subjects (infants) given two 10⁶ PFU doses given 29 days apart. A serum nAb response was detected in 78% of subjects, a serum IgG response was detected in 56% of subjects, and a nasal IgA response was detected in 44% of subjects, for a combined vaccine take of 89%.

FIG. 18 shows shedding of vaccine virus in subjects (infants) administered one or two doses of vaccine at 10⁶ PFU, as measured by plaque assay or PCR. Only 3 of 22 infants shed virus.

FIG. 19A shows the interim serum nAb results for 9 seronegative children (6-36 months of age) who received 2 doses at 10⁷ PFU of MV-012-968. The graph shows individual subjects, median, and interquartile range. As shown, administration of MV-012-968 leads to an increase in RSV-specific serum neutralizing antibody (nAb) titers.

FIG. 19B shows comparative safety, immunogenicity, and vaccine shedding data of different RSV live attenuated vaccines (LAV). Each dot represents a different RSV LAV candidate studied in seronegative infants and children in Phase 1. MV-012-968 Phase 1 data (green) are compared to prior, published candidates. Red, candidates with poor safety (e.g., LRI, cough). Orange, candidates with moderate safety (e.g., fever). Blue and green, candidates that were well-tolerated. Compared to previous RSV LAVs, two doses, 28 days apart, of 10⁶ PFU of MV-012-968 LAV was safe, immunogenic, with very low levels of shedding in seronegative pediatric subjects 6-36 months of age.

### DETAILED DESCRIPTION

Before the present disclosure is described in greater detail, it is to be understood that this disclosure is not limited to particular embodiments described, and as such may, of course, vary.

It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of immunology, medicine, organic chemistry, biochemistry, molecular biology, pharmacology, physiology, and the like, which are within the skill of the art. Such techniques are explained fully in the literature.

It must be noted that, as used in the specification and the appended claims, the singular forms “a,” “an,” and “the” include plural referents unless the context clearly dictates otherwise.

In this specification and in the claims that follow, reference will be made to a number of terms that shall be defined to have the following meanings unless a contrary intention is apparent.

Prior to describing the various embodiments, the following definitions are provided and should be used unless otherwise indicated.

The terms “protein” and “polypeptide” refer to compounds comprising amino acids joined via peptide bonds and are used interchangeably.

The term “portion” when used in reference to a protein (as in “a portion of a given protein”) refers to fragments of that protein. The fragments may range in size from four amino acid residues to the entire amino sequence minus one amino acid.

The term “chimera” when used in reference to a polypeptide refers to the expression product of two or more coding sequences obtained from different genes, that have been cloned together and that, after translation, act as a single polypeptide sequence. Chimeric polypeptides are also referred to as “hybrid” polypeptides. The coding sequences include those obtained from the same or from different species of organisms.

The term “homolog” or “homologous” when used in reference to a polypeptide refers to a high degree of sequence identity between two polypeptides, or to a high degree of similarity between the three-dimensional structure or to a high degree of similarity between the active site and the mechanism of action. In a preferred embodiment, a homolog has a greater than 60% sequence identity, and more preferably greater than 75% sequence identity, and still more preferably greater than 90% sequence identity, with a reference sequence.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity or more (e.g., 99 percent sequence identity). Preferably, residue positions which are not identical differ by conservative amino acid substitutions.

The terms “variant” and “mutant” when used in reference to a polypeptide refer to an amino acid sequence that differs by one or more amino acids from another, usually related polypeptide. The variant may have “conservative” changes, wherein a substituted amino acid has similar structural or chemical properties. One type of conservative amino acid substitutions refers to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. More rarely, a variant may have “non-conservative” changes (e.g., replacement of a glycine with a tryptophan). Similar minor variations may also include amino acid deletions or insertions (in other words, additions), or both. Guidance in determining which and how many amino acid residues may be substituted, inserted or deleted without abolishing biological activity may be found using computer programs well known in the art, for example, DNAStar software. Variants can be tested in functional assays. Preferred variants have less than 10%, and preferably less than 5%, and still more preferably less than 2% changes (whether substitutions, deletions, and so on).

The term “gene” refers to a nucleic acid (e.g., DNA or RNA) sequence that comprises coding sequences necessary for the production of an RNA, or a polypeptide or its precursor (e.g., pro insulin). A functional polypeptide can be encoded by a full length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. Thus, “a nucleotide comprising at least a portion of a gene” may comprise fragments of the gene or the entire gene.

The term “gene” also encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (mRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences which direct the termination of transcription, posttranscriptional cleavage and polyadenylation.

The term “heterologous gene” refers to a gene encoding a factor that is not in its natural environment (i.e., has been altered by the hand of man). For example, a heterologous gene includes a gene from one species introduced into another species. A heterologous gene also includes a gene native to an organism that has been altered in some way (e.g., mutated, added in multiple copies, linked to a non-native promoter or enhancer sequence, etc.). Heterologous genes are distinguished from endogenous plant genes in that the heterologous gene sequences are typically joined to nucleotide sequences comprising regulatory elements such as promoters that are not found naturally associated with the gene for the protein encoded by the heterologous gene or with plant gene sequences in the chromosome, or are associated with portions of the chromosome not found in nature (e.g., genes expressed in loci where the gene is not normally expressed).

The term “polynucleotide” refers to a molecule comprised of two or more deoxyribonucleotides or ribonucleotides, preferably more than three, and usually more than ten. The exact size will depend on many factors, which in turn depends on the ultimate function or use of the oligonucleotide. The polynucleotide may be generated in any manner, including chemical synthesis, DNA replication, reverse transcription, or a combination thereof. The term “oligonucleotide” generally refers to a short length of single-stranded polynucleotide chain usually less than 30 nucleotides long, although it may also be used interchangeably with the term “polynucleotide.”

The term “nucleic acid” refers to a polymer of nucleotides, or a polynucleotide, as described above. The term is used to designate a single molecule, or a collection of molecules. Nucleic acids may be single stranded or double stranded, and may include coding regions and regions of various control elements, as described below.

The term “a polynucleotide having a nucleotide sequence encoding a gene” or “a polynucleotide having a nucleotide sequence encoding a gene” or “a nucleic acid sequence encoding” a specified polypeptide refers to a nucleic acid sequence comprising the coding region of a gene or in other words the nucleic acid sequence which encodes a gene product. The coding region may be present in either a cDNA, genomic DNA or RNA form. When present in a DNA form, the oligonucleotide, polynucleotide, or nucleic acid may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present disclosure may contain endogenous enhancers/promoters, splice junctions, intervening sequences, polyadenylation signals, etc. or a combination of both endogenous and exogenous control elements.

The term “recombinant” when made in reference to a nucleic acid molecule refers to a nucleic acid molecule which is comprised of segments of nucleic acid joined together by means of molecular biological techniques. The term “recombinant” when made in reference to a protein or a polypeptide refers to a protein molecule which is expressed using a recombinant nucleic acid molecule.

The terms “complementary” and “complementarity” refer to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods which depend upon binding between nucleic acids.

The term “homology” when used in relation to nucleic acids refers to a degree of complementarity. There may be partial homology or complete homology (i.e., identity). “Sequence identity” refers to a measure of relatedness between two or more nucleic acids or proteins, and is given as a percentage with reference to the total comparison length. The identity calculation takes into account those nucleotide or amino acid residues that are identical and in the same relative positions in their respective larger sequences. Calculations of identity may be performed by algorithms contained within computer programs such as “GAP” (Genetics Computer Group, Madison, Wis.) and “ALIGN” (DNAStar, Madison, Wis.). A partially complementary sequence is one that at least partially inhibits (or competes with) a completely complementary sequence from hybridizing to a target nucleic acid is referred to using the functional term “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a sequence which is completely homologous to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target which lacks even a partial degree of complementarity (e.g., less than about 30% identity); in the absence of non-specific binding the probe will not hybridize to the second non-complementary target.

The following terms are used to describe the sequence relationships between two or more polynucleotides: “reference sequence”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA sequence given in a sequence listing or may comprise a complete gene sequence. Generally, a reference sequence is at least 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window”, as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman (Smith and Waterman, Adv. Appl. Math. 2: 482 (1981)) by the homology alignment algorithm of Needleman and Wunsch (Needleman and Wunsch, J. Mol. Biol. 48:443 (1970)), by the search for similarity method of Pearson and Lipman (Pearson and Lipman, Proc. Natl. Acad. Sci. (U.S.) 85:2444 (1988)), by computerized implementations of these algorithms (GAP, BESTFIT, FASTA, and TFASTA in the Wisconsin Genetics

Software Package Release 7.0, Genetics Computer Group, 575 Science Dr., Madison, Wis.), or by inspection, and the best alignment (i.e., resulting in the highest percentage of homology over the comparison window) generated by the various methods is selected. The term “sequence identity” means that two polynucleotide sequences are identical (i.e., on a nucleotide-by-nucleotide basis) over the window of comparison.

In certain embodiments, term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity.

In certain embodiments, sequence “identity” refers to the number of exactly matching amino acids (expressed as a percentage) in a sequence alignment between two sequences of the alignment calculated using the number of identical positions divided by the greater of the shortest sequence or the number of equivalent positions excluding overhangs wherein internal gaps are counted as an equivalent position. For example the polypeptides GGGGGG (SEQ ID NO: 25) and GGGGT (SEQ ID NO: 26) have a sequence identity of 4 out of 5 or 80%. For example, the polypeptides GGGPPP (SEQ ID NO: 27) and GGGAPPP (SEQ ID NO: 28) have a sequence identity of 6 out of 7 or 85%.

In certain embodiments, any recitation of sequence identity expressed herein may be substituted for sequence similarity. Percent “similarity” is used to quantify the similarity between two sequences of the alignment. This method is identical to determining the identity except that certain amino acids do not have to be identical to have a match. Amino acids are classified as matches if they are among a group with similar properties according to the following amino acid groups: Aromatic—F Y W; hydrophobic-A V I L; Charged positive: R K H; Charged negative−DE; Polar-STNQ.

The terms “substantial identity” as used herein denotes a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 25-50 nucleotides, wherein the percentage of sequence identity is calculated by comparing the reference sequence to the polynucleotide sequence which may include deletions or additions which total 20 percent or less of the reference sequence over the window of comparison. The reference sequence may be a subset of a larger sequence, for example, as a segment of the full-length sequences of the compositions claimed in the present disclosure.

When used in reference to a double-stranded nucleic acid sequence such as a cDNA or genomic clone, the term “substantially homologous” refers to any probe that can hybridize to either or both strands of the double-stranded nucleic acid sequence under conditions of low to high stringency as described above.

When used in reference to a single-stranded nucleic acid sequence, the term “substantially homologous” refers to any probe that can hybridize (i.e., it is the complement of) the single-stranded nucleic acid sequence under conditions of low to high stringency as described above.

The terms “in operable combination”, “in operable order” and “operably linked” refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “regulatory element” refers to a genetic element which controls some aspect of the expression of nucleic acid sequences. For example, a promoter is a regulatory element which facilitates the initiation of transcription of an operably linked coding region.

Other regulatory elements are splicing signals, polyadenylation signals, termination signals, etc.

Transcriptional control signals in eukaryotes comprise “promoter” and “enhancer” elements. Promoters and enhancers consist of short arrays of DNA sequences that interact specifically with cellular proteins involved in transcription (Maniatis, et al., Science 236: 1237, 1987). Promoter and enhancer elements have been isolated from a variety of eukaryotic sources including genes in yeast, insect, mammalian and plant cells. Promoter and enhancer elements have also been isolated from viruses and are found in prokaryotes. The selection of a particular promoter and enhancer depends on the cell type used to express the protein of interest. Some eukaryotic promoters and enhancers have a broad host range while others are functional in a limited subset of cell types (for review, see Voss, et al., Trends Biochem. Sci., 11:287, 1986; and Maniatis, et al., supra 1987).

The terms “promoter element,” “promoter,” or “promoter sequence” as used herein, refer to a DNA sequence that is located at the 5′ end (i.e. precedes) the protein coding region of a DNA polymer. The location of most promoters known in nature precedes the transcribed region.

The promoter functions as a switch, activating the expression of a gene. If the gene is activated, it is said to be transcribed, or participating in transcription.

Transcription involves the synthesis of mRNA from the gene. The promoter, therefore, serves as a transcriptional regulatory element and also provides a site for initiation of transcription of the gene into mRNA.

Promoters may be tissue specific or cell specific. The term “tissue specific” as it applies to a promoter refers to a promoter that is capable of directing selective expression of a nucleotide sequence of interest to a specific type of tissue (e.g., seeds) in the relative absence of expression of the same nucleotide sequence of interest in a different type of tissue (e.g., leaves). Tissue specificity of a promoter may be evaluated by, for example, operably linking a reporter gene to the promoter sequence to generate a reporter construct, introducing the reporter construct into the genome of a plant such that the reporter construct is integrated into every tissue of the resulting transgenic plant, and detecting the expression of the reporter gene (e.g., detecting mRNA, protein, or the activity of a protein encoded by the reporter gene) in different tissues of the transgenic plant. The detection of a greater level of expression of the reporter gene in one or more tissues relative to the level of expression of the reporter gene in other tissues shows that the promoter is specific for the tissues in which greater levels of expression are detected. The term “cell type specific” as applied to a promoter refers to a promoter which is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining. Briefly, tissue sections are embedded in paraffin, and paraffin sections are reacted with a primary antibody which is specific for the polypeptide product encoded by the nucleotide sequence of interest whose expression is controlled by the promoter. A labeled (e.g., peroxidase conjugated) secondary antibody which is specific for the primary antibody is allowed to bind to the sectioned tissue and specific binding detected (e.g., with avidin/biotin) by microscopy.

Promoters may be constitutive or regulatable. The term “constitutive” when made in reference to a promoter means that the promoter is capable of directing transcription of an operably linked nucleic acid sequence in the absence of a stimulus (e.g., heat shock, chemicals, light, etc.). Typically, constitutive promoters are capable of directing expression of a transgene in substantially any cell and any tissue.

In contrast, a “regulatable” or “inducible” promoter is one which is capable of directing a level of transcription of an operably linked nucleic acid sequence in the presence of a stimulus (e.g., heat shock, chemicals, light, etc.) which is different from the level of transcription of the operably linked nucleic acid sequence in the absence of the stimulus.

The enhancer and/or promoter may be “endogenous” or “exogenous” or “heterologous.” An “endogenous” enhancer or promoter is one that is naturally linked with a given gene in the genome. An “exogenous” or “heterologous” enhancer or promoter is one that is placed in juxtaposition to a gene by means of genetic manipulation (i.e., molecular biological techniques) such that transcription of the gene is directed by the linked enhancer or promoter. For example, an endogenous promoter in operable combination with a first gene can be isolated, removed, and placed in operable combination with a second gene, thereby making it a “heterologous promoter” in operable combination with the second gene. A variety of such combinations are contemplated (e.g., the first and second genes can be from the same species, or from different species).

Efficient expression of recombinant DNA sequences in eukaryotic cells typically requires expression of signals directing the efficient termination and polyadenylation of the resulting transcript. Transcription termination signals are generally found downstream of the polyadenylation signal and are a few hundred nucleotides in length. The term “poly(A) site” or “poly(A) sequence” as used herein denotes a DNA sequence which directs both the termination and polyadenylation of the nascent RNA transcript. Efficient polyadenylation of the recombinant transcript is desirable, as transcripts lacking a poly(A) tail are unstable and are rapidly degraded.

The poly(A) signal utilized in an expression vector may be “heterologous” or “endogenous.” An endogenous poly(A) signal is found naturally at the 3′ end of the coding region of a given gene in the genome. A heterologous poly(A) signal is one which has been isolated from one gene and positioned 3′ to another gene. A commonly used heterologous poly(A) signal is the SV40 poly(A) signal. The SV40 poly(A) signal is contained on a 237 bp BamHIBclI restriction fragment and directs both termination and polyadenylation.

The term “vector” refers to nucleic acid molecules that transfer DNA segment(s) from one cell to another. The term “vehicle” is sometimes used interchangeably with “vector.”

The terms “expression vector” or “expression cassette” refer to a recombinant nucleic acid containing a desired coding sequence and appropriate nucleic acid sequences used for the expression of the operably linked coding sequence in a particular host organism. Nucleic acid sequences used for expression in prokaryotes typically include a promoter, an operator (optional), and a ribosome binding site, often along with other sequences. Eukaryotic cells are known to utilize promoters, enhancers, and termination and polyadenylation signals.

The term “host cell” refers to any cell capable of replicating and/or transcribing and/or translating a heterologous gene. Thus, a “host cell” refers to any eukaryotic or prokaryotic cell (e.g., bacterial cells such as E. coli, yeast cells, mammalian cells, avian cells, amphibian cells, plant cells, fish cells, and insect cells), whether located in vitro or in vivo. For example, host cells may be located in a transgenic animal.

The term “selectable marker” refers to a gene which encodes an enzyme having an activity that confers antibiotic or drug resistance upon the cell in which the selectable marker is expressed, or which confers expression of a trait which can be detected (e.g., luminescence or fluorescence). Selectable markers may be “positive” or “negative.” Examples of positive selectable markers include the neomycin phosphotrasferase (NPTII) gene which confers resistance to G418 and to kanamycin, and the bacterial hygromycin phosphotransferase gene (hyg), which confers resistance to the antibiotic hygromycin. Negative selectable markers encode an enzymatic activity whose expression is cytotoxic to the cell when grown in an appropriate selective medium. For example, the HSV-tk gene is commonly used as a negative selectable marker. Expression of the HSV-tk gene in cells grown in the presence of gancyclovir or acyclovir is cytotoxic; thus, growth of cells in selective medium containing gancyclovir or acyclovir selects against cells capable of expressing a functional HSV TK enzyme.

The term “reporter gene” refers to a gene encoding a protein that may be assayed.

Examples of reporter genes include, but are not limited to, modified katushka, mkate and mkate2 (See, e.g., Merzlyak et al., Nat. Methods, 2007, 4, 555-557 and Shcherbo et al., Biochem. J., 2008, 418, 567-574), luciferase (See, e.g., deWet et al., Mol. Cell. Biol. 7:725 (1987) and U.S. Pat. Nos. 6,074,859; 5,976,796; 5,674,713; and 5,618,682; all of which are incorporated herein by reference), green fluorescent protein (e.g., GenBank Accession Number U43284; a number of GFP variants are commercially available from ClonTech Laboratories, Palo Alto, Calif), chloramphenicol acetyltransferase, beta-galactosidase, alkaline phosphatase, and horse radish peroxidase.

The term “wild-type” when made in reference to a gene refers to a gene which has the characteristics of a gene isolated from a naturally occurring source. The term “wild-type” when made in reference to a gene product refers to a gene product which has the characteristics of a gene product isolated from a naturally occurring source. The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring. A wild-type gene is that which is most frequently observed in a population and is thus arbitrarily designated the “normal” or “wild-type” form of the gene. In contrast, the term “modified” or “mutant” when made in reference to a gene or to a gene product refers, respectively, to a gene or to a gene product which displays modifications in sequence and/or functional properties (i.e., altered characteristics) when compared to the wild-type gene or gene product. It is noted that naturally-occurring mutants can be isolated; these are identified by the fact that they have altered characteristics when compared to the wild-type gene or gene product.

The term “antisense” or “antigenome” refers to a nucleotide sequence whose sequence of nucleotide residues is in reverse 5′ to 3′ orientation in relation to the sequence of nucleotide residues in a sense strand. A “sense strand” of a DNA duplex refers to a strand in a DNA duplex which is transcribed by a cell in its natural state into a “sense mRNA.” Thus an “antisense” sequence is a sequence having the same sequence as the non-coding strand in a DNA duplex.

The term “isolated” refers to a biological material, such as a virus, a nucleic acid or a protein, which is substantially free from components that normally accompany or interact with it in its naturally occurring environment. The isolated material optionally comprises material not found with the material in its natural environment, e.g., a cell. For example, if the material is in its natural environment, such as a cell, the material has been placed at a location in the cell (e.g., genome or genetic element) not native to a material found in that environment. For example, a naturally occurring nucleic acid (e.g., a coding sequence, a promoter, an enhancer, etc.) becomes isolated if it is introduced by non-naturally occurring means to a locus of the genome (e.g., a vector, such as a plasmid or virus vector, or amplicon) not native to that nucleic acid. Such nucleic acids are also referred to as “heterologous” nucleic acids. An isolated virus, for example, is in an environment (e.g., a cell culture system, or purified from cell culture) other than the native environment of wild-type virus (e.g., the nasopharynx of an infected individual).

An “immunologically effective amount” of RSV is an amount sufficient to enhance an individual's (e.g., a human's) own immune response against a subsequent exposure to RSV. Levels of induced immunity can be monitored, e.g., by measuring amounts of neutralizing secretory and/or serum antibodies, e.g., by plaque neutralization, complement fixation, enzyme-linked immunosorbent, or microneutralization assay.

A “protective immune response” against RSV refers to an immune response exhibited by an individual (e.g., a human) that is protective against serious lower respiratory tract disease (e.g., pneumonia and/or bronchiolitis) when the individual is subsequently exposed to and/or infected with wild-type RSV.

Recombinant respiratory syncytial virus (RSV) with codon usage silent mutations in the nonstructural genes

Live-attenuated RSV vaccine candidates have two major hurdles, suboptimal immunogenicity in infants and suboptimal stability that leads to genetic reversion towards wild-type and shedding of revertants by vaccinees. The viral nonstructural (NS) proteins, NS1 and NS2, are unique and inhibit type I interferon and T cell responses. Mutating NS1/NS2 for vaccine enhances immunogenicity. However, previously developed NS1 and NS1/NS2 deletion/null mutant recombinant RSV strains are over-attenuated, and the NS2 null mutant is under-attenuated in vivo.

Mutants disclosed herein overcome the limitations of over-attenuation and instability. Mutants were generated with partial NS1 and NS2 function to bridge the attenuation-immunogenicity gap for a pediatric vaccine. Gene synthesis and the RSV BAC rescue system was used to generate NS1/NS2 mutants by altering codon usage across the NS1 and NS2 genes.

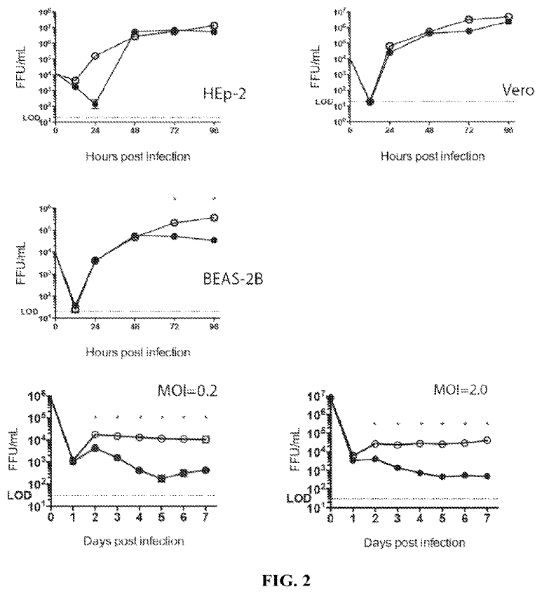

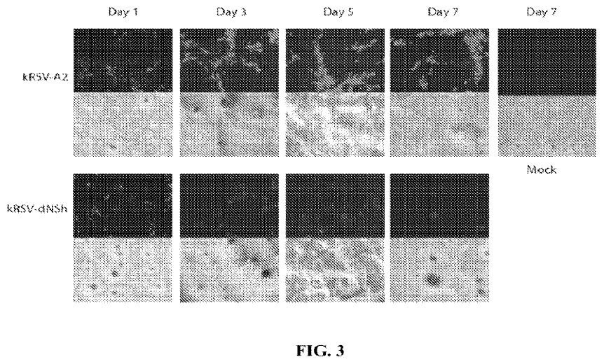

Codon de-optimization reduces translation efficiency by multiple mechanisms (e.g., tRNA concentration and mRNA structure). One mutant disclosed herein (“dNSh”) has 84/420 nt of NS1 mutated and 82/375 nt of NS2 mutated, reducing human codon preference without altering the amino acid sequences. This virus produces approximately 25% of wt NS1 levels, 25% of wt NS2 levels, 100% of wt nucleoprotein levels, and replicates like wt virus in Vero cells, the cell line commonly used to produce live attenuated RSV under GMP conditions (FIG. 2). In addition to reducing NS expression, this approach likely solves the genetic stability problem because there are too many mutations for reversion.

In certain embodiments, the disclosure relates to a vaccine, recombinant RSV genome, or an isolated recombinant nucleic acid encoding RSV NS1, NS2, N, P, M, G, F, M2-1, M2-2, and L genes comprising codon-deoptimization of the NS1 and NS2 genes, wherein codon-deoptimization is configured such that at least one codon to produce Gly is GGT, a codon to produce Asp is GAT, at least one codon to produce Glu is GAA, at least one codon to produce His is CAT, at least one codon to produce Ile is ATA, at least one codon to produce Lys is AAA, at least one codon to produce Leu is CTA, at least one codon to produce Asn is AAT, at least one codon to produce Gin is CAA, at least one codon to produce Val is GTA, or at least one codon to produce Tyr is TAT, wherein in greater than 25% of the Asp, Glu, His, Ile, Lys, Leu, Asn, Gin, Val, and Tyr amino acids are codon-deoptimized. In certain embodiments, greater than 75% of the amino acids are codon-deoptimized as compared to wild-type sequences, e.g., RSV A2 line 19.

In certain embodiments, the NS1 gene comprises (SEQ TD NO: 6) or variant thereof with greater than 70, 80, 90, 95, 97, 98, or 99% or more sequence identity thereto.

In certain embodiments, the NS2 gene comprises (SEQ ID NO: 9) or variant thereof with greater than 70, 80, 90, 95, 97, 98, or 99% or more sequence identity thereto.

In certain embodiments, the RSV small hydrophobic (SH) glycoprotein gene is deleted.

In certain embodiments, the nucleic acid has further codon-deoptimization of the G gene, wherein codon-deoptimization is configured such that at least one codon to produce Gly is GGT, a codon to produce Asp is GAT, at least one codon to produce Glu is GAA, at least one codon to produce His is CAT, at least one codon to produce Ile is ATA, at least one codon to produce Lys is AAA, at least one codon to produce Leu is CTA, at least one codon to produce Asn is AAT, at least one codon to produce Gin is CAA, at least one codon to produce Val is GTA, or at least one codon to produce Tyr is TAT, wherein in greater than 25% of the Asp, Glu, His, Ile, Lys, Leu, Asn, Gin, Val, and Tyr amino acids are codon-deoptimized.

In certain embodiments, the G gene comprises SEQ ID NO: 18

ATGTCGAAAAACAAAGACCAACGTACCGCGAAGACGTTAGAACGTACCTG
GGATACTCTAAATCATTTACTATTCATATCGTCGTGCCTATATAAGCTAA
ATCTTAAATCGGTAGCACAAATAACACTATCCATACTGGCGATAATAATC
TCGACTTCGCTTATAATAGCAGCGATCATATTTATAGCCTCGGCGAACCA
TAAAGTCACGCCAACGACTGCGATCATACAAGATGCGACATCGCAGATAA
AGAATACAACGCCAACGTACCTAACCCAAAATCCTCAACTTGGTATCTCG
CCCTCGAATCCGTCTGAAATAACATCGCAAATCACGACCATACTAGCGTC
AACGACACCGGGAGTAAAGTCGACCCTACAATCCACGACAGTAAAGACGA
AAAACACGACAACGACTCAAACGCAACCCTCGAAGCCGACCACGAAACAA
CGCCAAAATAAACCACCGAGCAAACCGAATAATGATTTTCACTTTGAAGT
ATTCAATTTTGTACCCTGTAGCATATGTAGCAATAATCCAACGTGCTGGG
CGATCTGTAAAAGAATACCGAACAAAAAACCGGGAAAAAAAACCACGACC
AAACCCACGAAAAAACCAACGCTCAAAACAACGAAAAAAGATCCCAAACC
GCAAACCACGAAATCAAAAGAAGTACCCACGACCAAACCCACGGAAGAGC
CGACCATAAACACGACCAAAACGAACATAATAACTACGCTACTCACGTCC
AATACCACGGGAAATCCGGAACTCACGAGTCAAATGGAAACGTTTCACTC
GACTTCGTCCGAAGGTAATCCATCGCCTTCGCAAGTCTCGACAACGTCCG
AATACCCGTCACAACCGTCATCGCCACCGAACACGCCACGTCAGTAG,

or variant thereof with greater than 70, 80, 90, 95, 97, 98, or 99% or more sequence identity thereto.

In certain embodiments, the Gene corn rises SEQ TD NO: 19

ATGTCGAAAAATAAAGACCAACGTACGGCGAAGACGCTAGAACGTACCTG
GGATACGCTAAATCATTTACTATTTATATCGTCGTGCCTATATAAACTAA
ATCTTAAATCGGTAGCGCAAATAACACTATCGATACTGGCGATAATAATA
TCGACTTCGCTAATAATAGCAGCGATAATATTTATAGCCTCGGCGAATCA
TAAAGTCACGCCGACGACTGCGATAATACAAGATGCGACATCGCAAATAA
AGAATACGACGCCAACGTATCTAACCCAAAATCCGCAACTTGGTATATCG
CCCTCGAATCCGTCGGAAATAACATCGCAAATAACGACCATACTAGCGTC
GACGACACCGGGTGTAAAGTCGACGCTACAATCCACGACGGTAAAGACGA
AAAATACGACAACGACGCAAACGCAACCGTCGAAACCGACCACGAAACAA
CGTCAAAATAAACCACCGTCGAAACCGAATAATGATTTTCACTTTGAAGT
ATTTAATTTTGTACCCTGTTCGATATGTAGCAATAATCCGACGTGCTGGG
CGATATGTAAAAGAATACCGAATAAAAAACCGGGAAAAAAAACGACGACC
AAACCGACGAAAAAACCAACGCTAAAAACAACGAAAAAAGATCCGAAACC
GCAAACCACGAAATCGAAAGAAGTACCCACGACGAAACCCACGGAAGAAC
CGACCATAAATACGACCAAAACGAATATAATAACTACGCTACTAACGTCC
AATACGACGGGAAATCCGGAACTAACGAGTCAAATGGAAACGTTTCATTC
GACTTCGTCGGAAGGTAATCCATCGCCGTCGCAAGTCTCGACGACTTCCG
AATATCCGTCACAACCGTCGTCGCCACCGAATACGCCACGTCAATAG,

or variant thereof with greater than 70, 80, 90, 95, 97, 98, or 99% or more sequence identity thereto.

In certain embodiments, the G gene comprises SEQ TD NO: 20

ATGTCGAAAAATAAAGATCAACGTACGGCGAAAACGCTAGAACGTACGTG
GGATACGCTAAATCATCTACTATTTATATCGTCGTGTCTATATAAACTAA
ATCTAAAATCGGTAGCGCAAATAACGCTATCGATACTAGCGATAATAATA
TCGACTTCGCTAATAATAGCGGCGATAATATTTATAGCGTCGGCGAATCA
TAAAGTAACGCCGACGACGGCGATAATACAAGATGCGACTTCGCAAATAA
AAAATACGACGCCGACGTATCTAACGCAAAATCCGCAACTAGGTATATCG
CCGTCGAATCCGTCGGAAATAACGTCGCAAATAACGACGATACTAGCGTC
GACGACGCCGGGTGTAAAATCGACGCTACAATCGACGACGGTAAAAACGA
AAAATACGACGACGACGCAAACGCAACCGTCGAAACCGACGACGAAACAA
CGTCAAAATAAACCGCCGTCGAAACCGAATAATGATTTTCATTTTGAAGT
ATTTAATTTTGTACCGTGTTCGATATGTTCGAATAATCCGACGTGTTGGG
CGATATGTAAACGTATACCGAATAAAAAACCGGGTAAAAAAACGACGACG
AAACCGACGAAAAAACCGACGCTAAAAACGACGAAAAAAGATCCGAAACC
GCAAACGACGAAATCGAAAGAAGTACCGACGACGAAACCGACGGAAGAAC
CGACGATAAATACGACGAAAACGAATATAATAACGACGCTACTAACGTCG
AATACGACGGGTAATCCGCAACTAACGTCGCAAATGGAAACGTTTCATTC
GACTTCGTCGGAAGGTAATCCGTCGCCGTCGCAAGTATCGACGACrTCGG
AATATCCGTCGCAACCGTCGTCGCCGCCGAATACGCCGCGTCAATAG.

or variant thereof with greater than 70, 80, 90, 95, 97, 98, or 99% or more sequence identity thereto.

In certain embodiments, F gene encodes a valine at position 557 and lysine at position 66. In certain embodiments, F gene encodes a valine at position 557 and the F gene comprises a sequence that encodes one or more of the follow amino acid sequences F gene comprises two, three, four, five or all of the follow amino acid sequences

(SEQ ID NO: 11)
TTNIMITTIIIVIIVILLSLIAVGLLLYCK,
(SEQ ID NO: 12)
ARSTPVPILKANAITTILAAVTFCFA,
(SEQ ID NO: 13)
AVTFCFASSQNITEEFYQST,
(SEQ ID NO: 14)
QSTCSAVSKGYLSALRTTWYTSVITIELSNIKK,
(SEQ ID NO: 15)
IKK NKCNGTDAKVKLMKQELDKYKNAV,
and
(SEQ ID NO: 16)
FPQAEKCKVQSNRVFC DTMYSLTLPSEVNLCNV.

In certain embodiments, the F gene comprises two, three, four, five or all of the follow amino acid sequences (SEQ ID NO: 11), (SEQ ID NO: 12), (SEQ ID NO: 13), (SEQ ID NO: 14), (SEQ ID NO: 15), and (SEQ ID NO: 16).

In certain embodiments, the F gene encodes a valine at position 557 and the F gene encodes one or more of the follow amino acids: asparagine at position 8, phenylalanine at position 20, serine at position 35, lysine at position 66, methionine at position 79, lysine at position 124, arginine at position 191, arginine at position 213, glutamic acid at position 354, lysine at position 357, tyrosine at position 371, valine at position 384, asparagine at position at 115, and threonine at position 523.

In certain embodiments, the F gene encodes a valine at position 557 and lysine at position 66 and methionine at position 79.

In certain embodiments, the F gene encodes a valine at position 557 and lysine at position 66 and arginine at position 191.

In certain embodiments, the F gene encodes a valine at position 557, lysine at position 66, arginine at position 191, and lysine at position 357.

In certain embodiments, the F gene encodes a valine at position 557, lysine at position 66, methionine at position 79, and asparagine at position at 115.

In certain embodiments, the F gene encodes SEQ ID NO: 17

MELPILKANAITTILAAVTPCPASSQNITEEFYQSTCSAVSKGYLSALRT
GWYTSVITIELSNIKKNKCNGTDAKVKLMKQELDKYKNAVTELQLLMQST
PAANNRARRELPRFMNYTLNNTKKTNVTLSKKRKRRFLGFLLGVGSAIAS
GIAVSKVLHLEGEVNKIKSALLSTNKAVVSLSNGVSVLTSRVLDLKNYID
KQLLPIVNKQSCRISNIETVIEFQQKNNRLLEITREFSVNAGVTTPVSTY
MLTNSELLSLINDMPITNDQKKLMSNNVQIVRQQSYSIMSIIKEEVLAYV
VQLPLYGVIDTPCWKLHTSPLCTTNTKEGSNICLTRTDRGWYCDNAGSVS
FFPQAEKCKVQSNRVFCDTMYSLTLPSEVNLCNVDIFNPKYDCKIMTSKT
DVSSSVITSLGAIVSCYGKTKCTASNKNRGHKTFSNGCDYVSNKGVDTVS
VGNTLYYVNKQEGKSLYVKGEPIINFYDPLVFPSDEFDASISQVNEKINQ
SLAFIRKSDELLHNVNAGKSTTNIMITTHIVIIVILLSLIAVGLLLYCKA
PRSTVTLSKDQLSGINNIAFSN

or variants that contain one, two, three, four, five, six, seven, eight, nine, or ten amino acid substitutions provided F gene encodes a valine at position 557. In certain embodiments, the amino acid substitutions are conservative substitutions.

In certain embodiments, the disclosure relates to an isolated recombinant nucleic acid comprising an F gene encoding (SEQ ID NO: 17) or variants that contains one or two amino acid substitutions provided F gene encodes a valine at position 557 and lysine at position 66.

In certain embodiments, the F gene encodes a valine at position 557 and the F gene encodes one or more of the follow amino acids: asparagine at position 8, phenylalanine at position 20, serine at position 35, lysine at position 66, methionine at position 79, lysine at position 124, arginine at position 191, arginine at position 213, glutamic acid at position 354, lysine at position 357, tyrosine at position 371, valine at position 384, asparagine at position at 115, and threonine at position 523.

In certain embodiments, the F gene encodes a valine at position 557 and lysine at position 66.

In certain embodiments, the F gene encodes a valine at position 557 and lysine at position 66 and methionine at position 79.

In certain embodiments, the F gene encodes a valine at position 557, lysine at position 66, arginine at position 191, and lysine at position 357.

In certain embodiments, the F gene encodes a valine at position 557, lysine at position 66, methionine at position 79, and asparagine at position at 115.

In certain embodiments, the disclosure relates to a recombinant vector comprising a nucleic acid disclosed herein. In certain embodiments, the disclosure relates to a cell comprising the recombinant vector, recombinant RSV, or attenuated recombinant RSV disclosed herein.

In certain embodiments, the disclosure relates to an F gene encoding (SEQ ID NO: 17) or variants that contains one amino acid substitutions provided F gene encodes a valine at position 557.

In certain embodiments, the disclosure relates to an F gene encoding

(SEQ ID NO: 21)
MELPILKANAITTILAAVTFCFASSQNITEEFYQSTCSAVSKGYLSALRT
GWYTSVITIELSNIKENKCNGTDAKVELMKQELDKYKNAVTELQLLMQST
PAANNRARRELPRFMNYTLNNTKKTNVTLSKKRKRRFLGFLLGVGSAIAS
GIAVSKVLHLEGEVNKIKSALLSTNKAVVSLSNGVSVLTSRVLDLKNYID
KQLLPIVNKQSCRISNIETVIEFQQKNNRLLEITREFSVNAGVTTPVSTY
MLTNSELLSLINDMPITNDQKKLMSNNVQIVRQQSYSIMSIIKEEVLAYV
VQLPLYGVIDTPCWKLHTSPLCTTNTKEGSNICLTRTDRGWYCDNAGSVS
FFPQAEKCKVQSNRVFCDTMYSLTLPSEVNLCNVDIFNPKYDCKIMTSKT
DVSSSVITSLGAIVSCYGKTKCTASNKNRGIIKTFSNGCDYVSNKGVDTV
SVGNTLYYVNKQEGKSLYVKGEPIINFYDPLVFPSDEFDASISQVNEKIN
QSLAFIRKSDELLHNVNAGKSTTNIMITTHIVIIVILLSLIAVGLLLYCK
ARSTPVTLSKDQLSGINNIAFSN.

In certain embodiments, the F gene encodes a valine at position 557 and glutamic acid at position 66 and arginine at position 191.

In certain embodiments, the disclosure relates to recombinant polypeptides comprising an RSV F protein sequence disclosed herein. In certain embodiments, the disclosure relates to virus particles or virus like particles produced by recombinant methods comprising a RSV F protein sequence disclosed herein.

In certain embodiments, the disclosure relates to an isolated recombinant nucleic acid comprising an RSV genome OE1 of SEQ ID NO: 1 or variant with greater than 70, 71, 72, 73, 74, 75, 76, 77, 78, 79, 80, 81, 82, 83, 84, 85, 86, 87, 88, 89, 90, 91, 92, 93, 94, 95, 96, 97, 98, or 9900 or more sequence identity thereto.

In certain embodiments, the disclosure relates to an isolated recombinant nucleic acid comprising an RSV genome OE2 of SEQ ID NO: 2 or variant with greater than 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% or more sequence identity thereto.

In certain embodiments, the disclosure relates to an isolated recombinant nucleic acid comprising an RSV genome OE3 of SEQ ID NO: 3 or variant with greater than 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% or more sequence identity thereto.

In certain embodiments, the disclosure relates to an isolated recombinant nucleic acid comprising an RSV genome OE4 of SEQ ID NO: 4 or variant with greater than 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% or more sequence identity thereto.

In certain embodiments, the disclosure contemplates isolated recombinant nucleic acids comprising RSV genomes OE1, OE2, OE3, and OE4, wherein one or both of the NS1 gene and NS2 gene are deleted.

### Cultivating RSV in a Bacterial Artificial Chromosome

Cultivating RSV in E. coli bacteria may be accomplished by utilizing a bacterial artificial chromosome (BAC). A BAC is disclosed that contains the complete antigenomic sequence of respiratory syncytial virus (RSV) strain A2 except the F gene, which is the antigenomic sequence of RSV strain line 19. Along with helper plasmids, it can be used in the reverse genetics system for the recovery of infectious virus. The antigenome sequence on the plasmid can be mutated prior to virus recovery to generate viruses with desired mutations.

The plasmid is an improvement on current RSV antigenomic plasmids for several reasons. Each RSV gene is flanked by restriction endonuclease cleavage sites to allow for easy manipulation of any gene. As a basis for viral mutagenesis, this plasmid may be used to design attenuated viruses for use in vaccines. An extra gene encoding the monomeric katushka 2, mKate2, protein has been included in the antigenome prior to the first RSV gene. The mKate2 protein is a far-red fluorescent protein which would be expressed in concert with the other RSV genes and would serve as visual evidence of virus replication. Changes have also been made to the ribozyme sequences that flank the RSV antigenome and play a role in the production of infectious virus through reverse genetics.

The disclosed vectors allow for efficient mutagenesis through recombineering. This mutagenesis method requires little to no ligation cloning, but relies on the recombination machinery present in bacteria harboring certain genes from a bacteriophage. Because RSV cDNAs are often unstable in mid-to-high copy number cloning vectors within bacteria predominantly used for cloning, such as Escherichia coli (E. coli), the single digit copy nature of the bacterial artificial chromosome reduces the instability, and the reduced instability is thought to occur because the single copy nature limits the ability E coli to recognize cryptic promoters in the RSV cDNA and produce toxic proteins.

### Respiratory Syncytial Virus (RSV)

Typically, the RSV particle contains a viral genome within a helical nucleocapsid which is surrounded by matrix proteins and an envelope containing viral glycoproteins. The genome of wild-type RSV encodes the proteins, NS1, NS2, N, P, M, SH, G, F, M2-1, M2-2, and L. G, F, and SH are glycoproteins. The F gene has been incorporated into a number of viral vaccines. RSV polymerase activity consists of the large protein (L) and phosphoprotein (P). The viral M2-1 protein is used during transcription and is likely to be a component of the transcriptase complex. The viral N protein is used to encapsidate the nascent RNA.

The genome is transcribed and replicated in the cytoplasm of a host cell. Host-cell transcription typically results in synthesis of ten methylated and polyadenylated mRNAs. The antigenome is positive-sense RNA complement of the genome produced during replication, which in turn acts as a template for genome synthesis. The viral genes are flanked by conserved gene-start (GS) and gene-end (GE) sequences. At the 3′ and 5′ ends of the genome are leader and trailer nucleotides. The wild type leader sequence contains a promoter at the 3′ end. When the viral polymerase reaches a GE signal, the polymerase polyadenylates and releases the mRNA and reinitiates RNA synthesis at the next GS signal. The L-P complex is believed to be responsible for recognition of the promoter, RNA synthesis, capping and methylation of the 5′ termini of the mRNAs and polyadenylation of their 3′ ends. It is believed that the polymerase sometimes dissociates from the gene at the junctions. Because the polymerase initiates transcription at the 3′ end of the genome, this results in a gradient of expression, with the genes at the 3′ end of the genome being transcribed more frequently than those at the 5′ end.

To replicate the genome, the polymerase does not respond to the cis-acting GE and GS signals and generates positive-sense RNA complement of the genome, the antigenome. At the 3′ end of the antigenome is the complement of the trailer, which contains a promoter. The polymerase uses this promoter to generate genome-sense RNA. Unlike mRNA, which is released as naked RNA, the antigenome and genome RNAs are encapsidated with virus nucleoprotein (N) as they are synthesized.

In certain embodiments, the disclosure relates to vectors and nucleic acids that contain RSV gene(s) such as the wild-type genome or antigenome. An example of an RSV antigenome is provided in U.S. Pat. No. 6,790,449, hereby incorporated by reference. Reference to RSV gene(s) and the genome is contemplated to include certain mutations, deletions, or variant combinations, such as cold-passaged (cp) and temperature sensitive (ts) derivatives of RSV, cpRSV, such as rA2cp248/404/1030ASH. rA2cp248/404ASH contains 4 independent attenuating genetic elements: cp which is based on 5 missense mutations in the N and L proteins and the F glycoprotein that together confer the non-ts attenuation phenotype of cpRSV; ts248, a missense mutation in the L protein; ts404, a nucleotide substitution in the gene-start transcription signal of the M2 gene; and ASH, complete deletion of the SH gene. rA2cp248/404/1030ASH contains 5 independent attenuating genetic elements: those present in rA2cp248/404ASH and ts1030, another missense mutation in the L protein. See Karron et al., J Infect Dis., 2005, 191(7): 1093-1104, hereby incorporated by reference. Within certain embodiments, it is contemplated that the RSV antigenome may contain deletion or mutations in nonessential genes (e.g., the SH, NS1, NS2, and M2-2 genes) or combinations thereof.

### Bacterial Artificial Chromosomes (BACs)

In certain embodiments, the disclosure relates to vectors and nucleic acids that contain bacterial artificial chromosomes. A bacterial cloning system for mapping and analysis of complex genomes has been disclosed in Shizuya et al., Proc. Natl. Acad. Sci., 1992, 89:8794-8797. The BAC system (for bacterial artificial chromosome) is based on Escherichia coli and its single-copy plasmid F factor which were described as useful for cloning large fragments of human DNA. The F factor encodes for genes that regulate its own replication including oriS, repE, parA, and parB. The oriS and repE genes mediate the unidirectional replication of the F factor while parA and parB typically maintain copy number at a level of one or two per E. coli genome. It is contemplated that the genes and the chromosome may contain mutations, deletions, or variants with desired functional attributes. The BAC vector (pBAC) typically contains these genes as well as a resistance marker and a cloning segment containing promoters for incorporating nucleic acid segments of interest by ligating into restriction enzyme sites. Exemplary BAC systems include those described in Shizuya & Kouros-Hehr, Keio J Med, 2001, 50(1): 26-30, hereby incorporated by reference.

One may reconstitute infectious RSV virus from the RSV BAC plasmids disclosed herein. BAC vectors can be transfected to bacteria such as E. coli by electroporation. The RSV-BACs disclosed herein may be stably maintained in bacteria, re-isolated from the bacteria, and inserted into a eukaryotic cell along with one or more vectors that express the N, P, L, and M2-1 proteins. These cells produce infective RSV particles. Production of infectious RSV results from co-transfection of plasmids encoding N, P, L, and M2-1 proteins and the antigenome under control of the T7 promoter into BHK-21 cells that express T7 RNA polymerase (BSR cells). See Buchholz et al., J Virol, 2000, 74(3): 1187-1199, hereby incorporated by reference.

### Vaccines

A number of attenuated RSV strains as candidate vaccines for intranasal administration have been developed using multiple rounds of chemical mutagenesis to introduce multiple mutations into a virus. Evaluation in rodents, chimpanzees, adults and infants indicate that certain of these candidate vaccine strains are immunogenic, and may be attenuated. Nucleotide sequence analysis of some of these attenuated viruses indicates that each level of increased attenuation is typically associated with two or more new nucleotide and amino acid substitutions.

The disclosure provides the ability to distinguish between silent incidental mutations versus those responsible for phenotype differences by introducing the mutations, separately and in various combinations, into the genome or antigenome of infectious RSV. This process identifies mutations responsible for phenotypes such as attenuation, temperature sensitivity, cold-adaptation, small plaque size, host range restriction, etc.

Mutations from this menu can then be introduced in various combinations to calibrate a vaccine virus to an appropriate level of attenuation, etc., as desired. Moreover, the present disclosure provides the ability to combine mutations from different strains of virus into one strain.

The present disclosure also provides for methods of attenuation. For example, individual internal genes of RSV can be replaced with their bovine, murine or other RSV counterpart. This may include part or all of one or more of the NS1, NS2, N, P, M, SH, M2-1, M2-2 and L genes, or parts of the G and F genes. Reciprocally, means are provided to generate a live attenuated bovine RSV by inserting human attenuating genes into a bovine RSV genome or antigenome background. Human RSV bearing bovine RSV glycoproteins provides a host range restriction favorable for human vaccine preparations. Bovine RSV sequences which can be used in the present disclosure are described in, e.g., Pastey et al., J. Gen. Viol. 76: 193-197 (1993); Pastey et al., Virus Res. 29: 195-202 (1993); Zamora et al., J. Gen. Virol. 73:737-741 (1992); Mallipeddi et al., J. Gen. Virol. 74:2001-2004 (1993); Mallipeddi et al., J. Gen. Virol. 73:2441-2444 (1992); and Zamora et al., Virus Res. 24: 115-121 (1992), each of which is incorporated herein by reference.

The disclosure also provides the ability to analyze other types of attenuating mutations and to incorporate them into infectious RSV for vaccine or other uses. For example, a tissue culture-adapted nonpathogenic strain of pneumonia virus of mice (the murine counterpart of RSV) lacks a cytoplasmic tail of the G protein (Randhawa et al., Virology 207: 240-245 (1995)). By analogy, the cytoplasmic and transmembrane domains of each of the RSV glycoproteins, F, G and SH, can be deleted or modified to achieve attenuation.

Other mutations for use in infectious RSV of the present disclosure include mutations in cis-acting signals identified during mutational analysis of RSV minigenomes. For example, insertional and deletional analysis of the leader and trailer and flanking sequences identified viral promoters and transcription signals and provided a series of mutations associated with varying degrees of reduction of RNA replication or transcription. Saturation mutagenesis (whereby each position in turn is modified to each of the nucleotide alternatives) of these cis-acting signals also has identified many mutations which reduced (or in one case increased) RNA replication or transcription. Any of these mutations can be inserted into the complete antigenome or genome as described herein. Other mutations involve replacement of the 3′ end of genome with its counterpart from antigenome, which is associated with changes in RNA replication and transcription. In addition, the intergenic regions (Collins et al., Proc. Natl. Acad. Sci. USA 83:4594-4598 (1986), incorporated herein by reference) can be shortened or lengthened or changed in sequence content, and the naturally-occurring gene overlap (Collins et al., Proc. Natl. Acad. Sci. USA 84:5134-5138 (1987), incorporated herein by reference) can be removed or changed to a different intergenic region by the methods described herein.

In another embodiment, RSV useful in a vaccine formulation can be conveniently modified to accommodate antigenic variation in circulating virus, including antigenic subgroup A and B strains and variations within those subgroups. Typically the modification will be in the G and/or F proteins. The entire G or F gene, or the segment(s) encoding particular immunogenic regions thereof, is incorporated into the RSV genome or antigenome cDNA by replacement of the corresponding region in the infectious clone or by adding one or more copies of the gene such that several antigenic forms are represented. Progeny virus produced from the modified RSV cDNA are then used in vaccination protocols against the emerging strains. Further, inclusion of the G protein gene of RSV subgroup B would broaden the response to cover a wider spectrum of the relatively diverse subgroup A and B strains infecting human populations.

An infectious RSV clone of the disclosure can also be engineered to enhance its immunogenicity and induce a level of protection greater than that provided by natural infection, or vice versa, to identify and ablate epitopes associated with undesirable immunopathologic reactions. Enhanced immunogenicity of the vaccines produced by the present disclosure addresses one of the greatest obstacles to controlling RSV, namely the incomplete nature of immunity induced by natural infection. An additional gene may be inserted into or proximate to the RSV genome or antigenome which is under the control of an independent set of transcription signals. Genes of interest include those encoding cytokines (e.g., IL-2 through IL-15, especially IL-3, IL-6 and IL-7, etc.), gamma-interferon, and proteins rich in T helper cell epitopes. The additional protein can be expressed either as a separate protein or as a chimera engineered from a second copy of one of the RSV proteins, such as SH. This provides the ability to modify and improve the immune response against RSV both quantitatively and qualitatively.

For vaccine use, virus produced according to the present disclosure can be used directly in vaccine formulations, or lyophilized, as desired, using lyophilization protocols well known to the artisan. Lyophilized virus will typically be maintained at about 4 degrees C. When ready for use the lyophilized virus is reconstituted in a stabilizing solution, e.g., saline or comprising SPG, Mg, and HEPES, with or without adjuvant, as further described below.

Thus RSV vaccines of the disclosure contain as an active ingredient an immunogenetically effective amount of RSV produced as described herein. The modified virus may be introduced into a host with a physiologically acceptable carrier and/or adjuvant. Useful carriers are well known in the art, and include, e.g., water, buffered water, 0.4% saline, 0.3% glycine, hyaluronic acid and the like. The resulting aqueous solutions may be packaged for use as is, or lyophilized, the lyophilized preparation being combined with a sterile solution prior to administration, as mentioned above. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, and the like. Acceptable adjuvants include incomplete Freund's adjuvant, aluminum phosphate, aluminum hydroxide, or alum, which are materials well known in the art.

Upon immunization with a RSV composition as described herein, via aerosol, droplet, oral, topical or other route, the immune system of the host responds to the vaccine by producing antibodies specific for RSV virus proteins, e.g., F and G glycoproteins. As a result of the vaccination the host becomes at least partially or completely immune to RSV infection, or resistant to developing moderate or severe RSV infection, particularly of the lower respiratory tract.

The host to which the vaccines are administered can be any mammal which is susceptible to infection by RSV or a closely related virus and which host is capable of generating a protective immune response to the antigens of the vaccinating strain. Thus, suitable hosts include humans, non-human primates, bovine, equine, swine, ovine, caprine, lagamorph, rodents, etc. Accordingly, the disclosure provides methods for creating vaccines for a variety of human and veterinary uses.

The vaccine compositions containing the RSV of the disclosure are administered to a host susceptible to or otherwise at risk of RSV infection to enhance the host's own immune response capabilities. Such an amount is defined to be an “immunogenically effective dose.” In this use, the precise amounts again depend on the host's state of health and weight, the mode of administration, the nature of the formulation. The vaccine formulations should provide a quantity of modified RSV of the disclosure sufficient to effectively protect the host patient against serious or life-threatening RSV infection.

The RSV produced in accordance with the present disclosure can be combined with viruses of the other subgroup or strains to achieve protection against multiple RSV subgroups or strains, or protective epitopes of these strains can be engineered into one virus as described herein. Typically the different viruses will be in admixture and administered simultaneously, but may also be administered separately. For example, as the F glycoproteins of the two RSV subgroups differ by only about 11% in amino acid sequence, this similarity is the basis for a cross-protective immune response as observed in animals immunized with RSV or F antigen and challenged with a heterologous strain. Thus, immunization with one strain may protect against different strains of the same or different subgroup.

In some instances it may be desirable to combine the RSV vaccines of the disclosure with vaccines which induce protective responses to other agents, particularly other childhood viruses. For example, the RSV vaccine of the present disclosure can be administered simultaneously with parainfluenza virus vaccine, such as described in Clements et al., J. Clin.

Microbiol. 29: 1175-1182 (1991), incorporated herein by reference. In another aspect of the disclosure the RSV can be employed as a vector for protective antigens of other respiratory tract pathogens, such as parainfluenza, by incorporating the sequences encoding those protective antigens into the RSV genome or antigenome which is used to produce infectious RSV as described herein.

Single or multiple administrations of the vaccine compositions of the disclosure can be carried out. In neonates and infants, multiple, sequential administrations may be required to elicit sufficient levels of immunity. Administration may begin within the first month of life, or before, about two months of age, typically not later than six months of age, and at intervals throughout childhood, such as at two months, six months, one year and two years, as necessary to maintain sufficient levels of protection against native (wild-type) RSV infection. Similarly, adults who are particularly susceptible to repeated or serious RSV infection, such as, for example, health care workers, day care workers, family members of young children, the elderly (over 55, 60, or 65 years), individuals with compromised cardiopulmonary function, may require multiple immunizations to establish and/or maintain protective immune responses. Levels of induced immunity can be monitored by measuring amounts of neutralizing secretory and serum antibodies, and dosages adjusted or vaccinations repeated as necessary to maintain desired levels of protection. Further, different vaccine viruses may be advantageous for different recipient groups. For example, an engineered RSV strain expressing an additional protein rich in T cell epitopes may be particularly advantageous for adults rather than for infants.

In yet another aspect of the disclosure, RSV is employed as a vector for transient gene therapy of the respiratory tract. According to this embodiment, the recombinant RSV genome or antigenome incorporates a sequence which is capable of encoding a gene product of interest. The gene product of interest is under control of the same or a different promoter from that which controls RSV expression. The infectious RSV produced by coexpressing the recombinant RSV genome or antigenome with the N, P, L and M2-1 proteins and containing a sequence encoding the gene product of interest is administered to a patient. Administration is typically by aerosol, nebulizer, or other topical application to the respiratory tract of the patient being treated. Recombinant RSV is administered in an amount sufficient to result in the expression of therapeutic or prophylactic levels of the desired gene product. Examples of representative gene products which are administered in this method include those which encode, for example, those particularly suitable for transient expression, e.g., interleukin-2, interleukin-4, gamma-interferon, GM-CSF, G-CSF, erythropoietin, and other cytokines, glucocerebrosidase, phenylalanine hydroxylase, cystic fibrosis transmembrane conductance regulator (CFTR), hypoxanthine-guanine phosphoribosyl transferase, cytotoxins, tumor suppressor genes, antisense RNAs, and vaccine antigens.

In certain embodiments, the disclosure relates to immunogenic compositions (e.g., vaccines) comprising an immunologically effective amount of a recombinant RSV of the invention (e.g., an attenuated live recombinant RSV or inactivated, non-replicating RSV), an immunologically effective amount of a polypeptide disclosed herein, and/or an immunologically effective amount of a nucleic acid disclosed herein.

In certain embodiments, the disclosure relates to methods for stimulating the immune system of an individual to produce a protective immune response against RSV. In the methods, an immunologically effective amount of a recombinant RSV disclosed herein, an immunologically effective amount of a polypeptide disclosed herein, and/or an immunologically effective amount of a nucleic acid disclosed herein is administered to the individual in a physiologically acceptable carrier.

Typically, the carrier or excipient is a pharmaceutically acceptable carrier or excipient, such as sterile water, aqueous saline solution, aqueous buffered saline solutions, aqueous dextrose solutions, aqueous glycerol solutions, ethanol, or combinations thereof. The preparation of such solutions ensuring sterility, pH, isotonicity, and stability is effected according to protocols established in the art. Generally, a carrier or excipient is selected to minimize allergic and other undesirable effects, and to suit the particular route of administration, e.g., subcutaneous, intramuscular, intranasal, oral, topical, etc. The resulting aqueous solutions can e.g., be packaged for use as is or lyophilized, the lyophilized preparation being combined with a sterile solution prior to administration

In certain embodiments, the RSV (or RSV components) is administered in an amount sufficient to stimulate an immune response specific for one or more strains of RSV (e.g., an immunologically effective amount of RSV or an RSV component is administered).

Preferably, administration of RSV elicits a protective immune response. For example, administration of the RSV (e.g., the live attenuated RSV vaccine) can result in at least one of an increase in RSV-specific serum neutralizing antibody (nAb) titers, an increase in serum binding (RSV F-specific) IgG concentrations, an increase in nasal mucosal binding (RSV F-specific) IgA concentrations, and reduced frequency of RSV-confirmed medically attended (1) acute respiratory infection and/or (2) lower respiratory infection.

In certain embodiments, virus is provided in the range of about 10³-10⁷ pfu (plaque forming units) per dose administered (e.g., 10⁶-10⁷ pfu per dose administered). Typically, the dose will be adjusted based on, e.g., age, physical condition, body weight, sex, diet, mode and time of administration, and other clinical factors.

The prophylactic vaccine formulation can be systemically administered, e.g., by subcutaneous or intramuscular injection using a needle and syringe or a needleless injection device. Preferably, the vaccine formulation is administered intranasally, e.g., by drops, aerosol (e.g., large particle aerosol (greater than about 10 microns)), spray into the upper respiratory tract, or a nasal atomizer. While any of the above routes of delivery results in a protective systemic immune response, intranasal administration confers the added benefit of eliciting mucosal immunity at the site of entry of the virus. For intranasal administration, attenuated live virus vaccines are often preferred, e.g., an attenuated, cold adapted and/or temperature sensitive recombinant RSV, e.g., a chimeric recombinant RSV.

In certain embodiments, the live attenuated RSV vaccine is administered to a child at a dose of from about 10³-10⁷ pfu (plaque forming units) per dose administered (e.g., 10⁵-10⁷ pfu per dose administered) intranasally over one or two doses. In certain embodiments, the child is between about 6 and 59 months of age. In certain embodiments, the child is between about 15 and 59 months of age. In certain embodiments, the child is between about 6 and 24 months of age. In certain embodiments, the child is between about 6 and 36 months of age. In certain embodiments, the live attenuated RSV vaccine comprises at least one of SEQ ID NO: 6, 9 and 20. In certain embodiments, the live attenuated RSV vaccine comprises SEQ ID NO: 6, 9 and 20. In certain embodiments, the live attenuated RSV vaccine comprises SEQ ID NO: 22. In certain embodiments, the intranasal administration is by drops or by nasal atomizer.

In certain embodiments, a child between about 6 and about 59 months of age (e.g., between about 6 and 24 months of age, between about 6 and 36 months of age, or between about 15 and 59 months of age) is intranasally administered a single live attenuated RSV vaccine dose at about 10³ pfu per dose. In certain embodiments, a child between about 6 and 24 months of age is intranasally administered a single live attenuated RSV vaccine dose at about 10⁴ pfu per dose. In certain embodiments, a child between about 6 and about 59 months of age (e.g., between about 6 and 24 months of age, between about 6 and 36 months of age, or between about 15 and 59 months of age) is intranasally administered a single live attenuated RSV vaccine dose at about 10⁵ pfu per dose. In certain embodiments, a child between about 6 and about 59 months of age (e.g., between about 6 and 24 months of age, between about 6 and 36 months of age, or between about 15 and 59 months of age) is intranasally administered a single live attenuated RSV vaccine dose at about 10⁶ pfu per dose. In certain embodiments, a child between about 6 and about 59 months of age (e.g., between about 6 and 24 months of age, between about 6 and 36 months of age, or between about 15 and 59 months of age) is intranasally administered a single live attenuated RSV vaccine dose at about 10⁷ pfu per dose. In certain embodiments, the live attenuated RSV vaccine comprises at least one of SEQ ID NO: 6, 9 and 20. In certain embodiments, the live attenuated RSV vaccine comprises SEQ ID NO: 6, 9 and 20. In certain embodiments, the live attenuated RSV vaccine comprises SEQ ID NO: 22. In certain embodiments, the intranasal administration is by drops or by nasal atomizer.

In certain embodiments, the vaccine is administered in two doses administered between about 3 and about 6 weeks apart (e.g., between about 3 and about 5 weeks apart, between about 3 and about 4 weeks apart, between about 4 and about 6 weeks apart, between about 4 and 5 weeks apart, or between about 5 and 6 weeks apart). In certain embodiments, the vaccine is administered in two doses administered between about 25 and 35 days apart, between about 25 and 30 days apart, between about 30 and 35 days apart, between about 28 and about 35 days apart, or between about 28 and about 32 days apart. In certain embodiments, the live attenuated RSV vaccine comprises at least one of SEQ ID NO: 6, 9 and 20. In certain embodiments, the live attenuated RSV vaccine comprises SEQ ID NO: 6, 9 and 20. In certain embodiments, the live attenuated RSV vaccine comprises SEQ ID NO: 22. In certain embodiments, the intranasal administration is by drops or by nasal atomizer.

In certain embodiments, a child between about 6 and about 59 months of age (e.g., between about 6 and 24 months of age, between about 6 and 36 months of age, or between about 15 and 59 months of age) is intranasally administered two doses of a live attenuated RSV vaccine at about 10³ pfu per dose, wherein the doses are administered between about 4 and 5 weeks apart (e.g., between about 28 and about 32 days apart). In certain embodiments, a child between about 6 and 24 months of age is intranasally administered two doses of a live attenuated RSV vaccine at about 10⁴ pfu per dose, wherein the doses are administered between about 4 and 5 weeks apart (e.g., between about 28 and about 32 days apart). In certain embodiments, a child between about 6 and 24 months of age is intranasally administered two doses of a live attenuated RSV vaccine at about 10⁵ pfu per dose, wherein the doses are administered between about 4 and 5 weeks apart (e.g., between about 28 and about 32 days apart). In certain embodiments, a child between about 6 and 24 months of age is intranasally administered two doses of a live attenuated RSV vaccine at about 10⁶ pfu per dose, wherein the doses are administered between about 4 and 5 weeks apart (e.g., between about 28 and about 32 days apart). In certain embodiments, a child between about 6 and 24 months of age is intranasally administered two doses of a live attenuated RSV vaccine at about 10⁷ pfu per dose, wherein the doses are administered between about 4 and 5 weeks apart (e.g., between about 28 and about 32 days apart). In certain embodiments, the live attenuated RSV vaccine comprises at least one of SEQ ID NO: 6, 9 and 20. In certain embodiments, the live attenuated RSV vaccine comprises SEQ ID NO: 6, 9 and 20. In certain embodiments, the live attenuated RSV vaccine comprises SEQ ID NO: 22. In certain embodiments, the intranasal administration is by drops or by nasal atomizer.

In certain embodiments, the attenuated recombinant RSV is as used in a vaccine and is sufficiently attenuated such that symptoms of infection, or at least symptoms of serious infection, will not occur in most individuals immunized (or otherwise infected) with the attenuated RSV in embodiments in which viral components (e.g., the nucleic acids or polypeptides herein) are used as vaccine or immunogenic components. However, virulence is typically sufficiently abrogated such that mild or severe lower respiratory tract infections do not typically occur in the vaccinated or incidental host.

While stimulation of a protective immune response with a single dose is preferred, additional dosages can be administered, by the same or different route, to achieve the desired prophylactic effect. In neonates and infants, for example, multiple administrations may be required to elicit sufficient levels of immunity. Administration can continue at intervals throughout childhood, as necessary to maintain sufficient levels of protection against wild-type RSV infection. Similarly, adults who are particularly susceptible to repeated or serious RSV infection, such as, for example, health care workers, day care workers, family members of young children, the elderly, and individuals with compromised cardiopulmonary function may require multiple immunizations to establish and/or maintain protective immune responses. Levels of induced immunity can be monitored, for example, by measuring amounts of virus-neutralizing secretory and serum antibodies, and dosages adjusted or vaccinations repeated as necessary to elicit and maintain desired levels of protection.

Alternatively, an immune response can be stimulated by ex vivo or in vivo targeting of dendritic cells with virus. For example, proliferating dendritic cells are exposed to viruses in a sufficient amount and for a sufficient period of time to permit capture of the RSV antigens by the dendritic cells. The cells are then transferred into a subject to be vaccinated by standard intravenous transplantation methods.

Optionally, the formulation for prophylactic administration of the RSV also contains one or more adjuvants for enhancing the immune response to the RSV antigens.

Suitable adjuvants include, for example: complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, bacille Calmette-Guerin (BCG), Corynebacterium parvum, and the synthetic adjuvant QS-21.

If desired, prophylactic vaccine administration of RSV can be performed in conjunction with administration of one or more immunostimulatory molecules.

Immunostimulatory molecules include various cytokines, lymphokines and chemokines with immunostimulatory, immunopotentiating, and pro-inflammatory activities, such as interleukins (e.g., IL-1, IL-2, IL-3, IL-4, IL-12, IL-13); growth factors (e.g., granulocyte-macrophage (GM)-colony stimulating factor (CSF)); and other immunostimulatory molecules, such as macrophage inflammatory factor, Flt3 ligand, B7.1; B7.2, etc. The immunostimulatory molecules can be administered in the same formulation as the RSV, or can be administered separately. Either the protein or an expression vector encoding the protein can be administered to produce an immunostimulatory effect.

Although vaccination of an individual with an attenuated RSV of a particular strain of a particular subgroup can induce cross-protection against RSV of different strains and/or subgroups, cross-protection can be enhanced, if desired, by vaccinating the individual with attenuated RSV from at least two strains, e.g., each of which represents a different subgroup. Similarly, the attenuated RSV vaccines can optionally be combined with vaccines that induce protective immune responses against other infectious agents.

### EXAMPLES

### Example 1

The following example describes the construction and testing of RSV viruses for use in a live attenuated RSV vaccine. The Example is derived from International Patent Publication No. WO2014152534, which is incorporated by reference herein in its entirety.

The A2-line19F-1557V virus is immunogenic in BALB/c mice

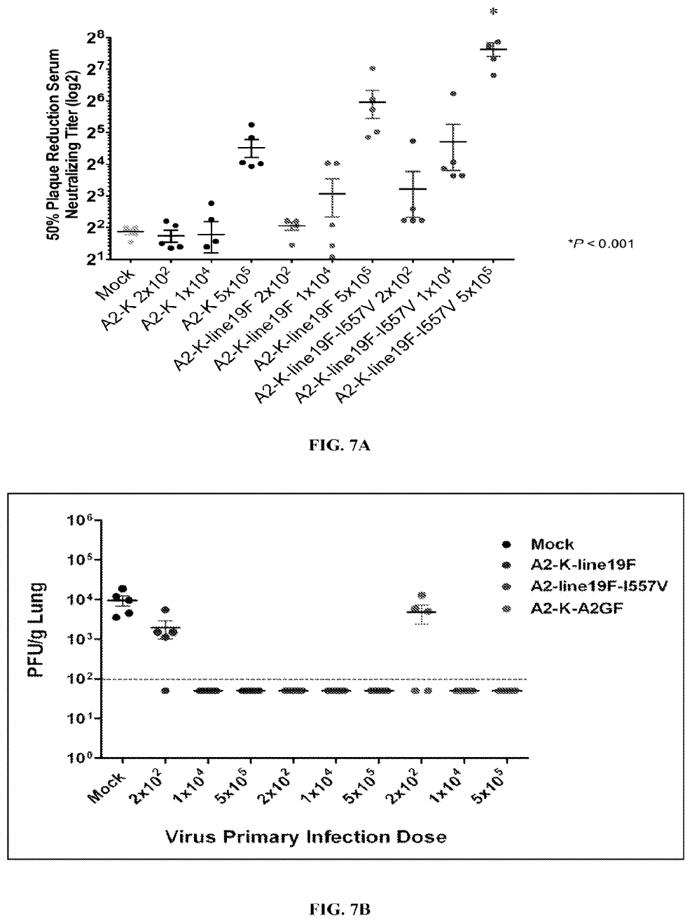

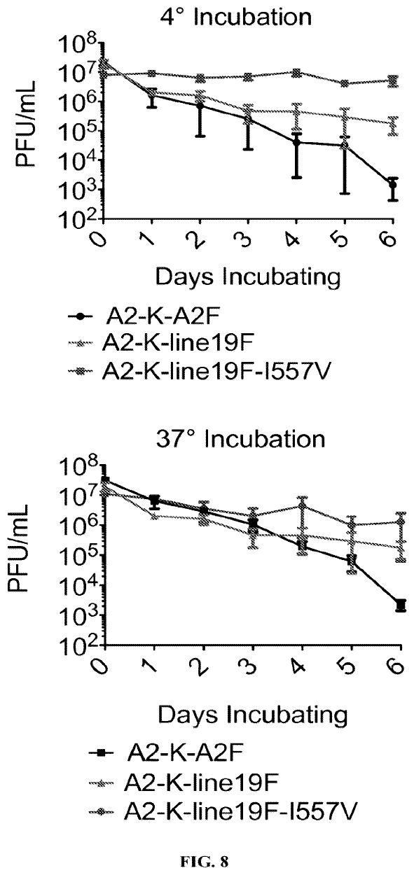

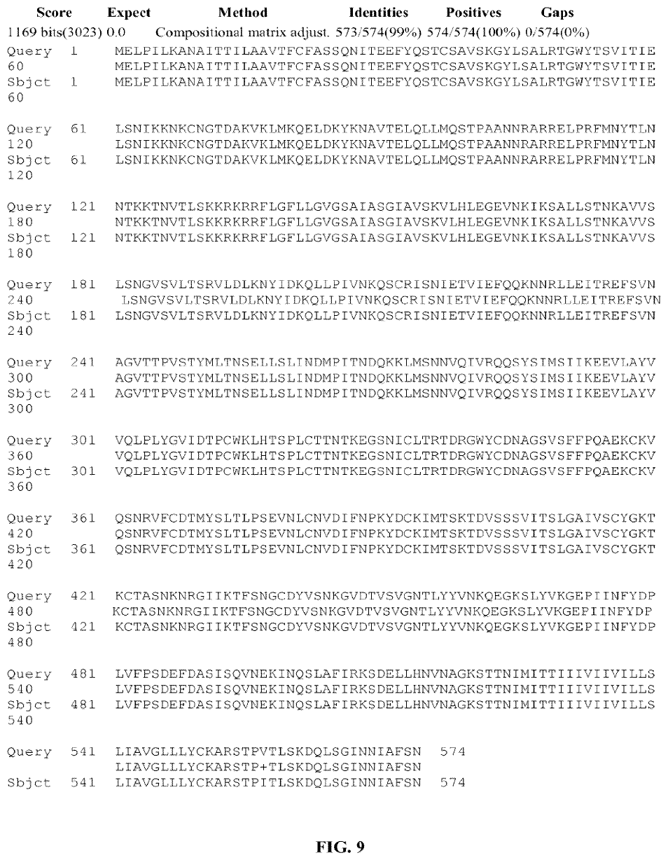

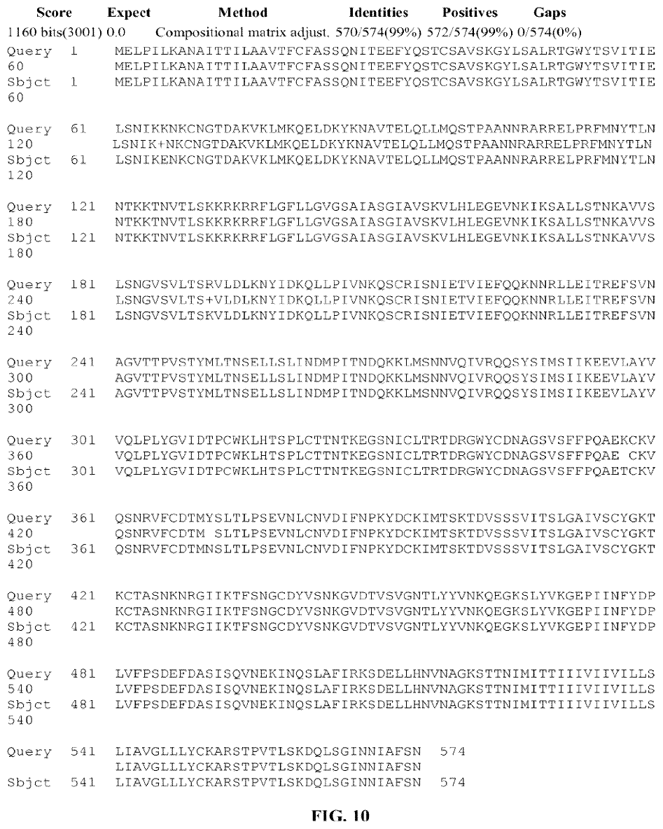

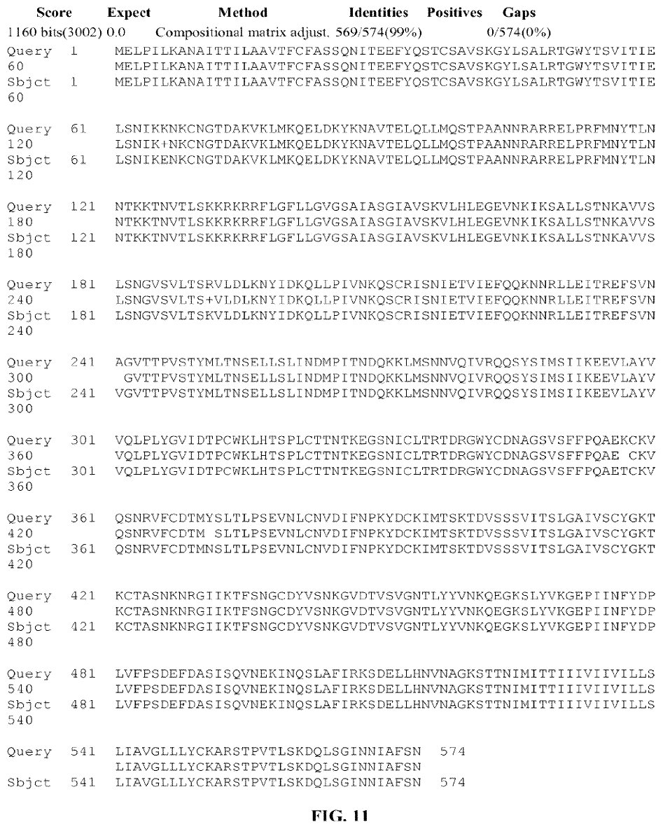

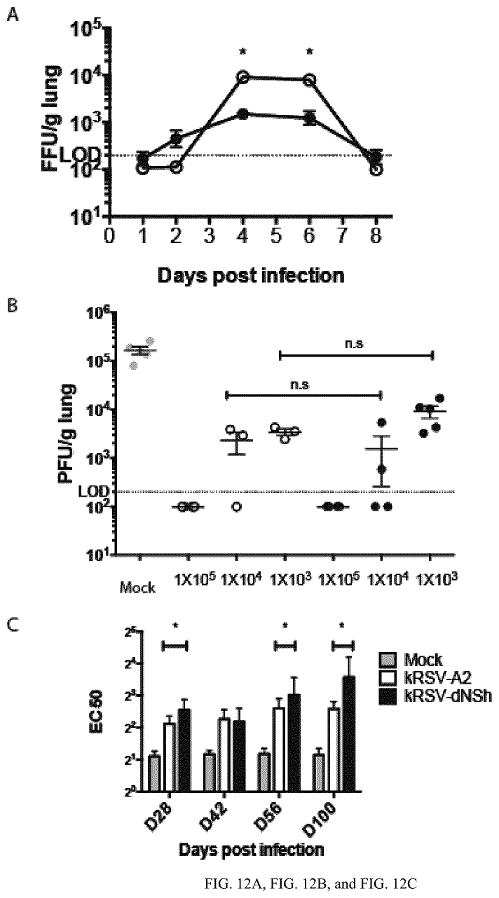

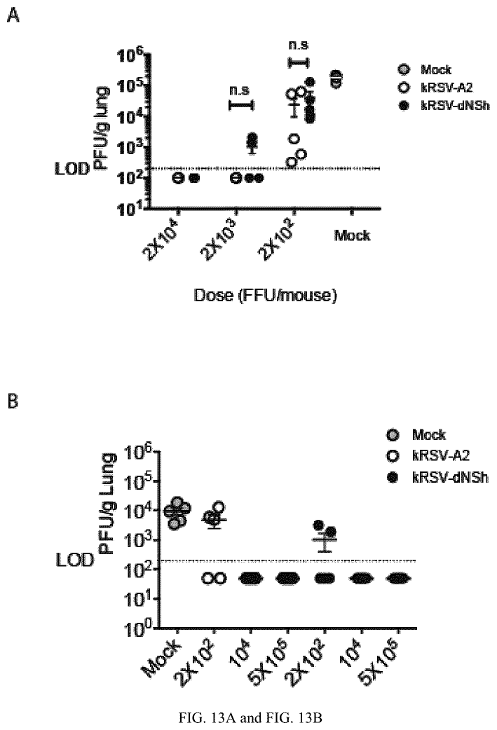

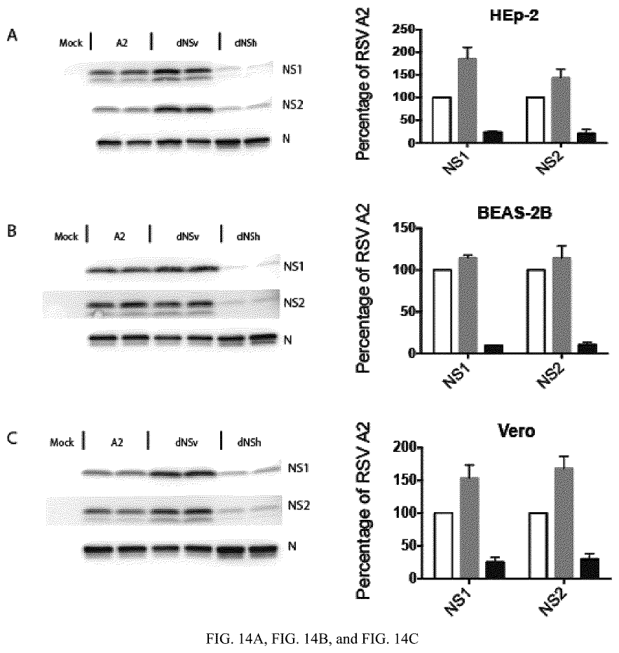

This is demonstrated in FIG. 7, which shows that this virus induces higher levels of RSV-neutralizing serum antibodies than RSV A2 and RSV A2-line19F. FIG. 7B demonstrates that, even low input doses, this virus provide complete protection to challenge with a heterologous strain of RSV, when challenged 29 days post-primary infection. This complete protection with low dose immunization is not seen for two other strains of RSV, A2-K-line19F and A2-K-A2GF, which allow for breakthrough reinfection. Those two viruses are similar to A2-line19F-I557V except for the F protein, indicating that the I557V F protein encoded by this virus is important for the phenotype.

In addition to being immunogenic (FIG. 7A), the A2-line19F-1557V virus is thermostable. Thermostability of the virus was measured as the ability of the virus to retain titer over multiple days when incubated at either 4° C. or 37° C. The results indicated indicate that this virus is more thermostable than the A2-K-A2GF virus at both temperatures tested and more stable than A2-line19F at 4° C. As stated above, the F gene is the only difference between these two viruses, indicating this unique F protein is responsible for the phenotype.

The A2-line 19 F RSV strain is more stable than the A2 strain, and Val at 557 in the context of the line 19 F protein makes the virus even more stable. Val at position 557 in other strains is also likely stabilizing. In certain embodiments, the disclosure contemplates other mutations at position 557 (any amino acid, e.g., alanine, valine, isoleucine, leucine), in any F strain context, that affect thermostability of the virus.

Generation of recombinant RSV with NS1 and NS2 codon silent mutations and Growth Attenuation

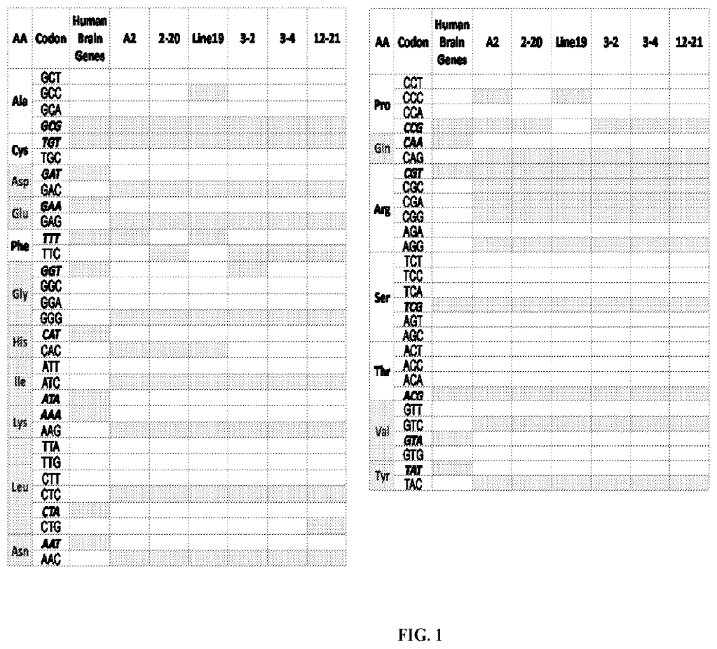

Codons that are uncommon in humans were used to prepare recombinant RSV with the NS1 and NS2 genes designated dNS1h and dNS2h below. Codons that are uncommon in RSV were used to prepare recombinant RSV with the NS1 and NS2 genes designated dNS1v and dNS2v below. FIG. 1 provides a table used to determine optimal sequences. Recombinant RSV was prepared with the following nucleotide sequences for the NS1 and NS2 gene. It is important to note that prior to testing codons, it was unpredictable if either the uncommon human codons or uncommon RSV codons would produce a desirable RSV vaccine candidate.

Experiments using codons uncommon for RSV sequences had the unanticipated and undesirable effect of increased expression. Using codons uncommon for human sequences had the desirable effect of decreased expression. Experiments comparing NS codons uncommon for human sequences and NS codons uncommon for RSV sequences indicated that the codons uncommon for human sequences were preferential for vaccine development.

dNS1h nucleotide sequence (SEQ ID NO: 6) has which as 84 out of 420 nucleotides (20%) different and 68 out of 140 codons (48%) than NS1 in wild-type A2

SEQ ID NO: 6
ATGGGTTCGAATTCGCTATCGATGATAAAAGTACGTCTACAAAATCTATT
TGATAATGATGAAGTAGCGCTACTAAAAATAACGTGTTATACGGATAAAC
TAATACATCTAACGAATGCGCTAGCGAAAGCGGTAATACATACGATAAAA
CTAAATGGTATAGTATTTGTACATGTAATAACGTCGTCGGATATATGTCC
GAATAATAATATAGTAGTAAAATCGAATTTTACGACGATGCCGGTACTAC
AAAATGGTGGTTATATATGGGAAATGATGGAACTAACGCATTGTTCGCAA
CCGAATGGTCTACTAGATGATAATTGTGAAATAAAATTTTCGAAAAAACT
ATCGGATTCGACGATGACGAATTATATGAATCAACTATCGGAACTACTAG
GTTTTGATCTAAATCCGTAA

dNS1v nucleotide sequence (SEQ ID NO. 7) has which as 145 out of 420 nucleotides (34%) different and 122 out of 140 codons (87%) than NS1 in wild-type A2

SEQ ID NO: 7
ATGGGGTCGAACTCGCTCTCGATGATCAAGGTCCGCCTCCAGAATCTCTT
CGACAACGACGAGGTCGCGCTCCTCAAGATCACGTGTTACACGGACAAGC
TCATCCACCTCACGAACGCGCTCGCCAAGGCGGTCATCCACACGATCAAG
CTCAACGGGATCGTCTTCGTCCACGTCATCACGTCGTCGGACATCTGTCC
GAACAACAACATCGTCGTCAAGTCGAACTTCACGACGATGCCGGTCCTCC
AGAACGGGGGGTACATCTGGGAGATGATGGAGCTCACGCACTGTTCGCAG
CCGAACGGGCTCCTCGACGACAACTGTGAGATCAAGTTCTCGAAGAAGCT
CTCGGACTCGACGATGACGAACTACATGAACCAGCTCTCGGAGCTCCTCG
GGTTCGACCTCAACCCGTAA

dNS2h nucleotide sequence (SEQ ID NO: 9) has which as 82 out of 420 nucleotides (21%) different and 73 out of 140 codons (58%) than NS1 in wild-type A2

SEQ ID NO: 9
ATGGATACGACGCATAATGATAATACGCCGCAACGTCTAATGATAACGGA
TATGCGTCCGCTATCGCTAGAAACGATAATAACGTCGCTAACGCGTGATA
TAATAACGCATAAATTTATATATCTAATAAATCATGAATGTATAGTACGT
AAACTAGATGAACGTCAAGCGACGTTTACGTTTCTAGTAAATTATGAAAT
GAAACTACTACATAAAGTAGGTTCGACGAAATATAAAAAATATACGGAAT
ATAATACGAAATATGGTACGTTTCCGATGCCGATATTTATAAATCATGAT
GGTTTTCTAGAATGTATAGGTATAAAACCGACGAAACATACGCCGATAAT
TATAAAATATGATCTAAATCCGTAA

dNS2v nucleotide sequence (SEQ ID NO: 10) has which as 10³ out of 420 nucleotides (27%) different and 92 out of 140 codons (73%) than NS1 in wild-type A2

SEQ ID NO: 10
ATGGACACGACGCACAACGACAACACGCCCGCAGCGCCTCATGATCACGG
ACATGCGCCCGCTCTCGCTCGAGACGATCATCACGTCGCTCACGCGCGAC
ATCATCACGCACAAGTTCATCTACCTCATCAACCACGAGTGTATCGTCCG
CAAGCTCGACGAGCGCCAGGCGACGTTCACGTTCCTCGTCAACTACGAGA
TGAAGCTCCTCCACAAGGTCGGGTCGACGAAGTACAAGAAGTACACGGAG
TACAACACGAAGTACGGGACGTTCCCGATGCCGATCTTCATCAACCACGA
CGGGTTCCTCGAGTGTATCGGGATCAAGCCGACGAAGCACACGCCGATCA
TCTACAAGTACGACCTCAACCCGTAA

BEAS-2B cell lines at 60-70% confluence are infected with the recombinant virus indicated as above at MOI (multiplicity of infection) of 0.01 (i.e., for each 100 cells, there is one infectious virus particle). This is done by first counting the cells before infection, calculating the total number of cells in each well, then calculating the amount of each virus for infection.

Infection is done at room temperature for 1 hour, then washed off. The infected cells are left in 370 C incubator with 5% CO2 for up to 96 hours. Samples are taken at 12, 24, 48, 72, and 96 hours after infection and frozen. After collecting all the time point samples, the amount of virus in each sample is determined by titering on Vero cell lines according to standard protocol and the titer (FFU/mL, meaning Fluorescent Focus-forming Unit per mL) is calculated for each sample. Since viruses used have a red fluorescent gene in the genome, the infected cells are counted under the fluorescent microscope providing fluorescent focus-forming units. Each data point represents duplicate samples from two independent experiments.

As illustrated in FIG. 2, growth of kRSV-dNS1h (human deoptimized NS1+NS2 virus) is attenuated in the BEAS-2B cell line at 72 and 96 hours post infection. It is believed that this is due to lower NS1 and NS2 proteins than wild type virus.

### Expression of RSV in Plasmid Designed for Low Copy Number

Infectious recombinant RSV (rRSV) can be recovered from transfected plasmids.

Co-expression of RSV N, P, L, and M2-1 proteins as well as the full-length antigenomic RNA is sufficient for RSV replication. Infectious RSV may be produced from the co-transfection of plasmids encoding N, P, L, and M2-1 proteins and the antigenomic cDNA under control of the T7 promoter into BHK-21 cells that stably express T7 RNA polymerase (BSR cells). Currently research labs typically use a RSV antigenomic cDNA cloned in the plasmid pBR322 (mid-range copy number, 15-20 copies per E coli). In order to maintain the antigenomic cDNA in this plasmid, the bacteria is grown at 300 C and low aeration. Nevertheless, plasmid rearrangements and clone loss is frequently experienced.

A fraction of RSV cDNA containing the attachment glycoprotein (G) and fusion (F) genes of the virus was found to be unclonable in pUC-based plasmids (500-700 plasmid copies in E coli). This fragment was cloned in a low copy number (approximately 5 copies per E. coli) plasmid called pLG338-30.5. The plasmid pLG338-30 was developed to increase the stability of cloned lentivirus glycoproteins. Cunningham et al., Gene, 1993, 124, 93-98. It is hypothesized that cDNA instability in E coli results from the presence of cryptic E coli transcription promoters within viral glycoprotein sequences. Thus, instability of cDNA in “promoterless” plasmids in bacteria can arise because aberrant proteins are expressed from cryptic promoters, leading to toxicity exacerbated by plasmid copy number.

An antigenomic plasmid was generated containing the RSV strain A2 genome with the strain line 19 F gene in place of the A2 F gene. It had been derived from the antigenome plasmid first disclosed in Collins et al., Proc Natl Acad Sci USA., 1995, 92(25): 11563-1 1567 and U.S. Pat. No. 6,790,449 hereby incorporated by reference. The antigenome was digested out of the plasmid vector and ligated into the pKBS3 BAC.

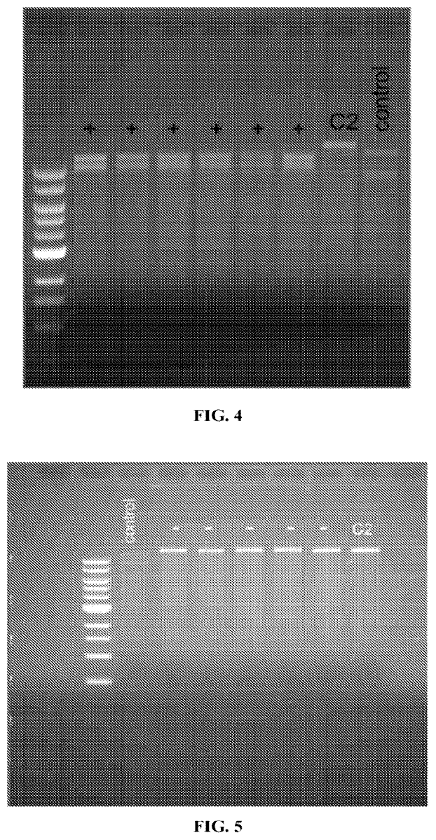

GalK recombineering reagents were obtained from the NCI and successfully established a BAC-RSV reverse genetics protocol (FIGS. 4 and 5). See the website web.ncifcrf.gov/research/brb/recombineeringlnformation.aspx, hereby incorporated by reference. Mutation of RSV cDNA via BAC recombineering has enhanced the ability to manipulate RSV for generation of mutants. An added benefit of the system is enhanced stability of the full-length antigenomic cDNA in the BAC vector.

The BAC-based RSV antigenome vector was propagated at 320 C and 250 RPM without observing any vector rearrangements or clone loss in E coli. Thus, BAC-RSV not only enables manipulations via recombineering but also facilitates RSV reverse genetics in general owing to elimination of cDNA instability.

RSV Antigenome in BAC Vector (pSynkRSV_Line 19 F Construction)

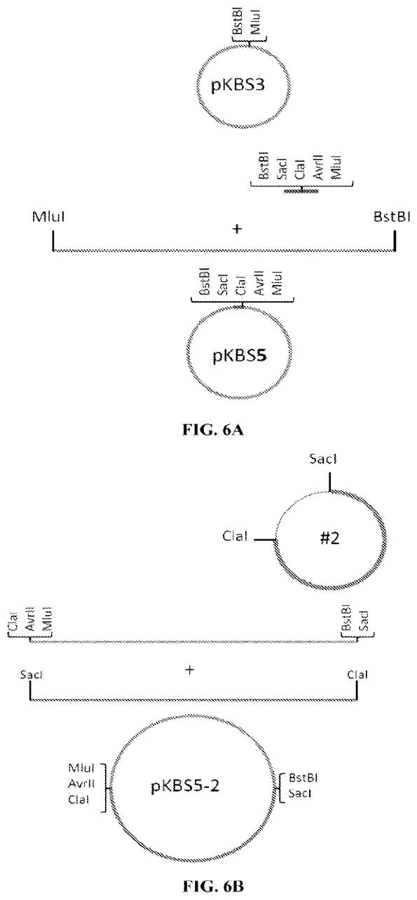

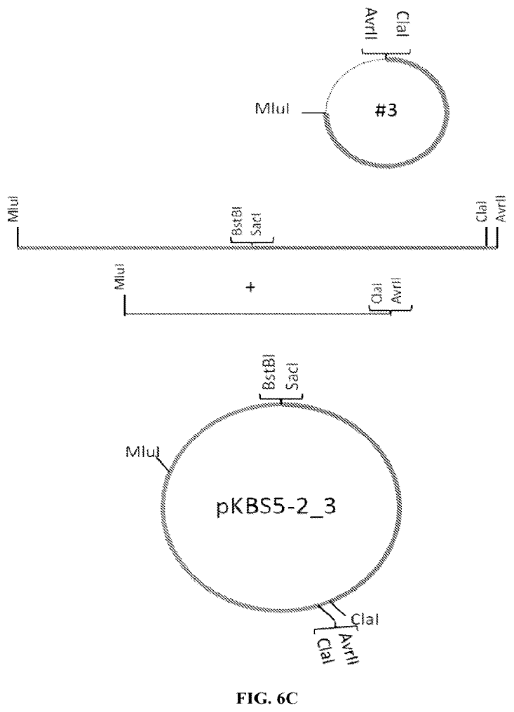

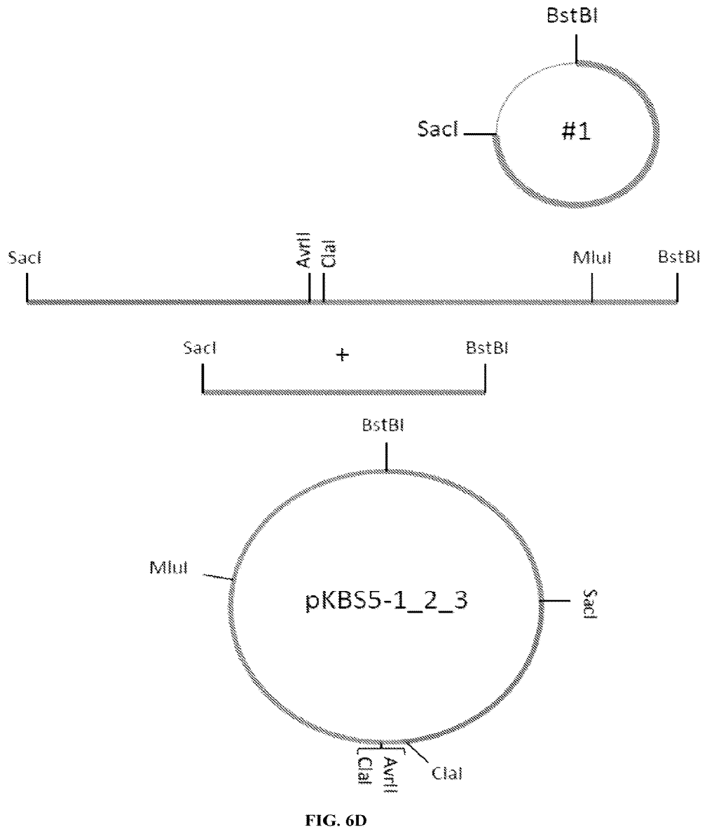

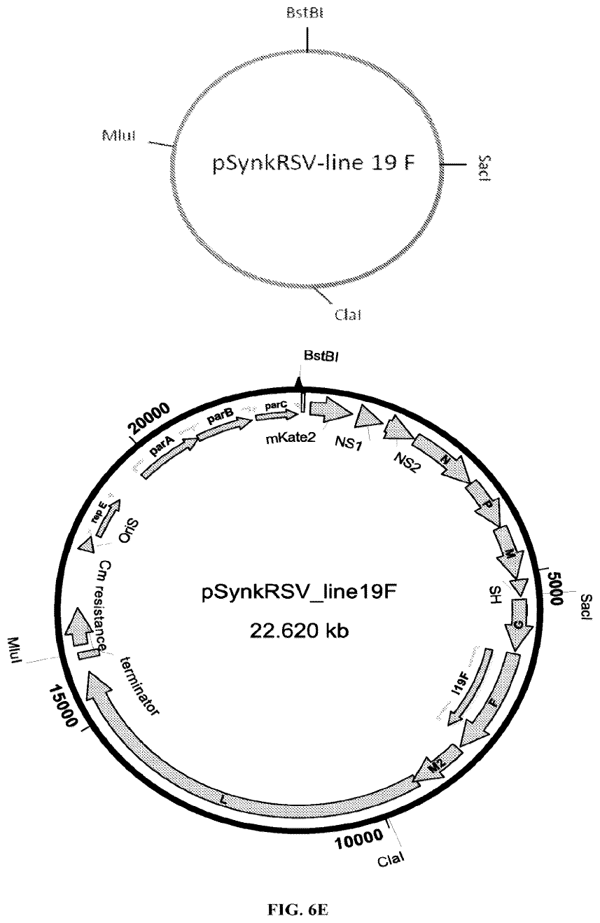

The RSV-BAC pSynkRSV_line 19 F contains the modified katushka gene (mKate2, fluorescent protein), and restriction sites for convenient standard cloning methods. To build pSynkRSV, three nucleic acid pieces were synthesized by Gene Art, a company that synthesizes DNA. These three pieces then have to be put together in the bacterial artificial chromosome (BAC). The three pieces are designated pSynkRSV-BstBI_SacI (#1), pSynkRSV-SacI_ClaI (#2), and pSynkRSV-ClaI_MluI (#3). One uses the plasmid pKBS3 as the backbone for constructing pSynkRSV. See FIGS. 6A-E. pSynkRSV contains the bacterial artificial chromosome sequences needed to regulate copy number and partitioning in the bacteria.

To insert the three synthesized segments, one puts oligonucleotide adapters into pKBS3 between two existing restriction enzyme cut sites, BstBI and MluI.

The overhangs were designed such that the adapter would ligate into pKBS3 at the BstBI and MluI sites. Underlined sequences indicate restriction sites: SacI, ClaI, and AvrII from right to left respectively. This produces a multi-cloning site containing the restriction sites BstBI, SacI, ClaI, AvrII, and MluI, in that order, and a plasmid termed pKBS5. See FIG. 6A. One cuts and ligates the SacI_ClaI segment (#2) from Gene Art into pKBS5. See FIG. 6B. The next one cuts and ligates the #3 segment using the enzymes AvrII and MluI (cannot use ClaI again due to an inactive ClaI restriction site in pSynkRSV-ClaI_MluI). See FIG. 6C. At this point, the plasmid pKBS5 contains the Gene Art sequences from SacI to ClaI, some intervening nucleotides (less than 10), and the Gene Art sequences from AvrII to MluI. One cuts and ligates the #1 segment using BstBI and SacI. See FIG. 6D. This RSV BAC contains about 10 unwanted nucleotides between two ClaI sites (that from segment #2 and segment #3). Recombineering is used to delete those nucleotides, thus generating pSynkRSV_line 19 F. See FIG. 6E. The three segments should be ligated in this order to avoid potential interference from multiple restriction sites.

### Recombinant Respiratory Syncytial Virus (RSV) as Live-Attenuated Vaccine (LAV)

Four expression plasmids were generated, one that expresses RSV nucleoprotein (N), one that expresses RSV phosphoprotein (P), one that expresses RSV matrix 2 ORF 1 protein (M2-1), and one that expresses RSV large polymerase (L)-pA2-Nopt, pA2-Popt, pA2-M2-1opt, and pA2-Lopt. The nomenclature reflects the fact that these genes are of the A2 strain of RSV and that these cDNAs are optimized for human codon bias in order to increase expression levels in mammalian cells. Recovery of recombinant RSV from cDNA includes five components: full length RNA (e.g. provided by pSynk-RSV119F), and RSV N, P, M2-1, and L proteins. The four helpers plasmids pA2-Nopt, pA2-Popt, pA2-M2-lopt, and pA2-Lopt useful for driving RSV rescue.

A recombinant respiratory syncytial virus strain A2-line19F was generated with a point mutation at residue F557, at which the isoleucine was changed to a valine (virus name: A2-line19F-I557V). A protein expression plasmid was also generated which encodes the line 19 F protein with the same isoleucine to valine mutation at position 557 (protein name-line 19F-I557V). A2-line19F-1557V has higher thermostability, at 4° C. and 370 C, than the A2-line 19F parent virus. This increased stability likely contributes to an increased induction of neutralizing antibodies and protection by A2-line19F-1557V relative to A2-line 19F.

Development of a live-attenuated RSV vaccine has been hindered by low RSV immunogenicity in young infants, which constitute the target population, and limited genomic stability. A desirable vaccine is immunogenic and genetically and thermally stable and safe for vaccination in young infants.

RSV nonstructural (NS) proteins 1 and 2 (NS1 and NS2) are associated with inhibition of host cell interferon pathways and thus potentially limiting the immunogenicity of the virus. The small hydrophobic (SH) glycoprotein forms cationic pores in membranes, modulates the host apoptotic pathways and inhibits tumor necrosis factor-a (TNF-α) signaling. SH, NS1 and NS2 are dispensable for virus replication. However, deletion of NS1 and NS2 together results in an over-attenuation. Deletion of the SH protein has little apparent effect on attenuation in experimental vaccine candidates currently being evaluated. However, deletion of SH enhances RSV replication in vitro and presumably enhances expression of downstream genes, such as the antigenic G and F genes.

RSV vaccine candidates disclosed herein combine multiple technologies to overcome the challenges of poor immunogenicity and limited genetic and thermal stability in a safe viral vaccine candidate. RSV LAV OE1 combines limited expression of immune inhibitory proteins NS1 and NS2 through codon-deoptimization and SH protein through deletion without the potential for rapid reversion in a stable and immunogenic viral background.

Vaccine candidates were generated using BAC-based RSV reverse genetics codon-deoptimization of nonstructural (NS) genes NS1 and NS2 were combined with the A2-line 19F gene containing a mutation at residue 557, as well as deletion of the RSV small hydrophobic (SH) glycoprotein.
OE1 Virus Genome (SEQ ID NO: 1)
RSV vaccine candidate genotype:
   A2-mKate2-dNSh-deltaSH-A2G-line19F-1557V (tagged)
   and A2-dNSh-deltaSH-A2G-line19F-1557V (untagged)

RSV attachment glycoprotein (G) is a heavily glycosylated protein, which exists in two variant forms: membrane-bound and secreted. Studies evaluating the functional role of RSV G have shown that it plays a role in inhibition of toll-like receptor activation and its secreted form likely acts as an immune antigen decoy. In addition to RSV F, G protein is also immunogenic, however due in part to its extensive glycosylation, it is a poor antigen for generation of neutralizing antibodies. RSV G is indispensable for virus replication, but deletion results in over-attenuation. Thus, G can be considered a non-essential virulence gene.

An RSV A2 G protein sequence was substituted which contains a M48I mutation and has 50% of the codons deoptimized [dGm(50%)] into the background of the RSV LAV OE1 virus genome. The OE2 virus background includes codon-deoptimization of nonstructural (NS) genes NS1 and NS2 with the A2-line 19F gene containing a mutation at amino acid residue 557, as well as deletion of the RSV small hydrophobic (SH) glycoprotein.
OE2 Virus Genome (SEQ ID NO: 2)
RSV vaccine candidate genotype:
   A2-mKate2-dNSh-deltaSH-dGm(50%)-line19F-1557V (tagged)
   and A2-dNSh-deltaSH-dGm(50%)-line19F-1557V (untagged)

RSV LAV OE2 combines reduced expression of immune inhibitory glycoprotein G through codon-deoptimization of 50% of codons, 100% codon-deoptimization of immunomodulatory proteins NS1 and NS2, and deletion of SH protein without the potential for rapid reversion in a stable and immunogenic viral background.

In a third vaccine candidate, an RSV A2 G protein sequence substituted with one which contains a M48I mutation and has 75% of the codons deoptimized [dGm(75%)] into the background of the RSV LAV OE1 virus genome. The OE3 virus background includes codon-deoptimization of nonstructural (NS) genes NS1 and NS2 with the A2-line 19F gene containing a mutation at residue 557, as well as deletion of the RSV small hydrophobic (SH) glycoprotein.
OE3 Virus Genome (SEQ ID NO: 3)
RSV vaccine candidate genotype:
   A2-mKate2-dNSh-deltaSH-dGm(75%)-line19F-I557V (tagged)
   and A2-dNSh-deltaSH-dGm(75%)-line19F-I557V (untagged)

RSV LAV OE3 combines reduced expression of immune inhibitory glycoprotein G through codon-deoptimization of 75% of codons, 100% codon deoptimization of immunomodulatory proteins NS1 and NS2, and deletion of SH protein without the potential for rapid reversion in a stable and immunogenic viral background.

An RSV A2 G protein sequence which contains a M48I mutation and has 100% of the codons deoptimized [dGm(100%)] into the background of the RSV LAV OE1 virus genome was generated. The OE4 virus background includes codon-deoptimization of nonstructural (NS) genes NS1 and NS2 with the A2-line 19F gene containing a mutation at residue 557, as well as deletion of the RSV small hydrophobic (SH) glycoprotein.
OE4 Virus Genome (SEQ ID NO: 4)
RSV vaccine candidate genotype:
   A2-mKate2-dNSh-deltaSH-dGm(100%)-line19F-I557V (tagged)
   and A2-dNSh-deltaSH-dGm(100%)-line19F-1557V (untagged)

### Example 2

This study evaluates a vaccine that is designed to protect humans against infection with respiratory syncytial virus (RSV) and is administered as a nasal spray. Specifically, the study analyzes the safety of, and the immune response to, the vaccine when administered to healthy children between the ages of 6 and 36 months who are seronegative to RSV.

The first 5 enrolled participants were assigned to Dosage Group 1 and randomly allocated to receive either vaccine (genome sequence provided at SEQ ID NO: 22) at Dosage 1 (10³ PFU/dose) or placebo. The next 10 enrolled participants were assigned to Dosage Group 2 and randomly allocated to receive either investigational vaccine at Dosage 2 (10⁴ PFU/dose) or placebo. The next 24 enrolled participants were assigned to Dosage Group 3 and randomly allocated to receive investigational vaccine at Dosage 3 (10⁵ PFU/dose) or placebo; of these 24, 12 participants, received a second, identical dose of investigational vaccine or placebo 29 days after the initial dose. The next 24 enrolled participants were assigned to Dosage Group 4 and randomly allocated to receive investigational vaccine at Dosage 4 (10⁶ PFU/dose) or placebo; of these 24, 12 participants, received a second, identical dose of investigational vaccine or placebo 29 days after the initial dose. The next 10-15 enrolled participants will be assigned to Dosage Group 5 and randomly allocated to receive two doses of investigational vaccine at Dosage 5 (10⁷ PFU/dose) or placebo, administered 29 days apart.

TABLE 1

| Arms | Assigned Interventions |
|---|---|
| Experimental: Dosage Group 1: | Biological/Vaccine: Investigational |
| RSV Vaccine Dosage 1 | RSV vaccine MV-012-968 (Dosage 1) |
| Participants in this arm will | Single dose administered intranasally |
| receive a single intranasal | on Day 1 |
| dose of the investigational | |
| RSV vaccine at Dosage 1 | |
| Experimental: Dosage Group 2: | Biological/Vaccine: Investigational |
| RSV Vaccine Dosage 2 | RSV vaccine MV-012-968 (Dosage 2) |
| Participants in this arm will | Single dose administered intranasally |
| receive a single intranasal | on Day 1 |
| dose of the investigational | |
| RSV vaccine at Dosage 2 | |
| Experimental: Dosage Group 3: | Biological/Vaccine: Investigational |
| RSV Vaccine Dosage 3 (Single- | RSV vaccine MV-012-968 (Dosage 3; |
| dose) | single-dose) |
| Participants in this arm will | Single dose administered intranasally |
| receive a single intranasal | on Day 1 |
| dose of the investigational | |
| RSV vaccine at Dosage 3 | |
| Experimental: Dosage Group | Biological/Vaccine: Investigational |
| 3a: RSV Vaccine Dosage 3 | RSV vaccine MV-012-968 (Dosage 3; |
| (Two-dose) | two-dose) |
| Participants in this arm will | Single dose administered intranasally |
| receive a single intranasal | on Day 1, followed by an identical |
| dose of the investigational | dose administered intranasally at |
| RSV vaccine at Dosage 3 | the Day 30 study visit |
| followed by a second identical | |
| dose of the investigational | |
| RSV vaccine 29 days later | |
| Experimental: Dosage Group 4: | Biological/Vaccine: Investigational |
| RSV Vaccine Dosage 4 (Single- | RSV vaccine MV-012-968 (Dosage 4; |
| dose) | single-dose) |
| Participants in this arm will | Single dose administered intranasally |
| receive a single intranasal | on Day 1 |
| dose of the investigational | |
| RSV vaccine at Dosage 4 | |
| Experimental: Dosage Group | Biological/Vaccine: Investigational |
| 4a: RSV Vaccine Dosage 4 | RSV vaccine MV-012-968 (Dosage 4; |
| (Two-dose) | two-dose) |
| Participants in this arm will | Single dose administered intranasally |
| receive a single intranasal | on Day 1, followed by an identical |
| dose of the investigational | dose administered intranasally at |
| RSV vaccineat Dosage 4 | the Day 30 study visit |
| followed by a second identical | |
| dose of the investigational | |
| RSV vaccine 29 days later | |
| Experimental: Dosage Group | Biological/Vaccine: Investigational |
| 5: RSV Vaccine Dosage 5 | RSV vaccine MV-012-968 (Dosage 5; |
| (Two-dose) | two-dose) |
| Participants in this arm will | Single dose administered intranasally |
| receive a single intranasal | on Day 1, followed by an identical |
| dose of the investigational | dose administered intranasally at |
| RSV vaccine at Dosage 5 | the Day 30 study visit |
| followed by a second identical | |
| dose of the investigational | |
| RSV vaccine 29 days later | |
| Placebo Comparator: Placebo | Placebo (single-dose) |
| (Single-dose) | Single dose administered intranasally |
| Participants in this arm will | on Day 1 |
| receive a single intranasal | |
| dose of placebo | |
| Placebo Comparator: Placebo | Placebo (two-dose) |
| (Two-dose) | Single dose administered intranasally |
| Participants in this arm will | on Day 1, followed by an identical |
| receive a single intranasal | dose administered intranasally at |
| dose of placebo followed by | the Day 30 study visit |
| a second identical dose of | |
| placebo 29 days later | |

### Outcome Measures

The Primary Outcome Measures included solicited adverse events (AEs), unsolicited AEs, serious adverse events (SAEs), medically attended adverse events (MAEs), and change in RSV-specific serum neutralizing antibody (nAb) titers (GMT). Solicited and unsolicited AEs were determined in the period immediately post-vaccination. SAEs and MAEs are being determined throughout the full study duration (about 1 year). The change in RSV-specific serum neutralizing antibody (nAb) titers (GMT) was determined from baseline through day 28, an average of six (6) weeks.

The frequency of solicited AEs are being measured, categorized by severity. Solicited AEs are predefined AEs that may occur after investigational vaccine administration.

The frequency of unsolicited AEs are being measured, categorized by severity. Unsolicited AEs are any untoward medical occurrences in a participant administered the investigational vaccine, regardless of causal relationship to the investigational vaccine.

Unsolicited AEs can include unfavorable and unintended signs (including abnormal laboratory findings), symptoms, or diseases temporally associated with the use of the investigational vaccine.

The frequency of SAEs are being measured, categorized by vaccine-relatedness. SAEs are AEs, whether considered causally related to the investigational vaccine or not, that threaten life or result in any of the following: death, inpatient hospitalization or prolongation of existing hospitalization, persistent or significant incapacity or substantial disruption of the ability to conduct normal life functions, or congenital anomaly/birth defect.

The frequency of MAEs are being measured, categorized by vaccine-relatedness. MAEs are AEs, whether considered causally related to the investigational vaccine or not, with unscheduled medically attended visits, such as urgent care visits, acute primary care visits, emergency department visits, or other previously unplanned visits to a medical provider. Scheduled medical visits such as routine physicals, wellness checks, ‘check-ups’, and vaccinations, are not considered MAEs.

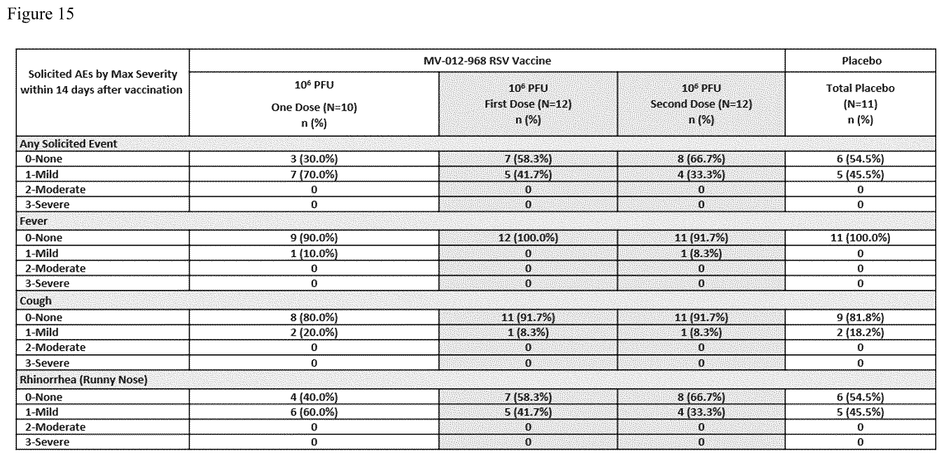

AEs occurring within 14 days of vaccinations are depicted in FIG. 15. As shown, no SAEs were observed, and AEs were generally mild and unrelated to the study vaccine.

No evidence of lower respiratory tract symptoms was seen. The change in RSV-specific serum neutralizing antibody (nAb) titers (GMT) Change in serum RSV-specific neutralizing antibody (nAb) titers were measured per participant.

Secondary Outcome Measures included (1) neutralizing antibody (nAb) concentrations, (2) change in serum binding (RSV F-specific) IgG concentrations, (2) change in nasal mucosal binding (RSV F-specific) IgA concentrations, and (3) frequency, magnitude, and duration of potential vaccine virus shedding after a single intranasal dose of MV-012-968.

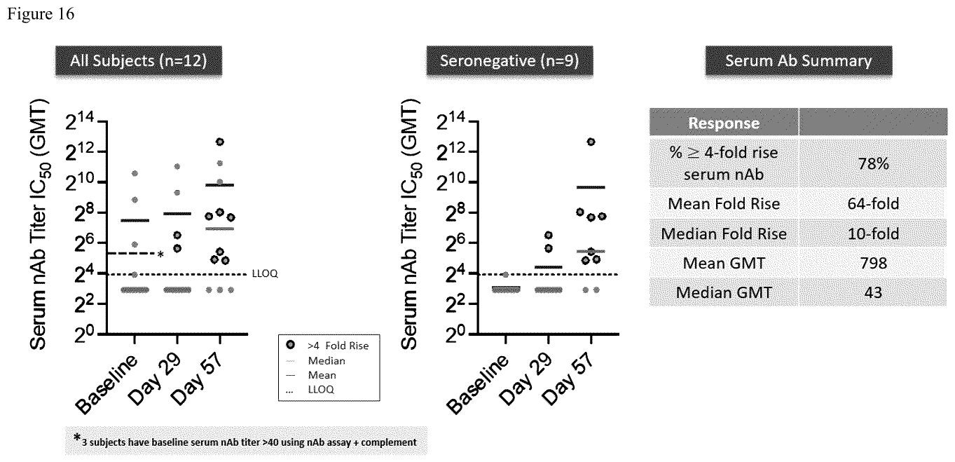

As shown in FIG. 16, two 10⁶ PFU doses given 29 days apart stimulated serum nAb response in 7 of 9 seronegative infants. Seventy-eight (78) percent of infants showed a greater than 4-fold rise in serum nAb, with a mean fold rise in nAb of 64-fold, a median fold rise in nAb of 10-fold, and mean geometric mean titer (GMT) of 798 and a median GMT of 43.

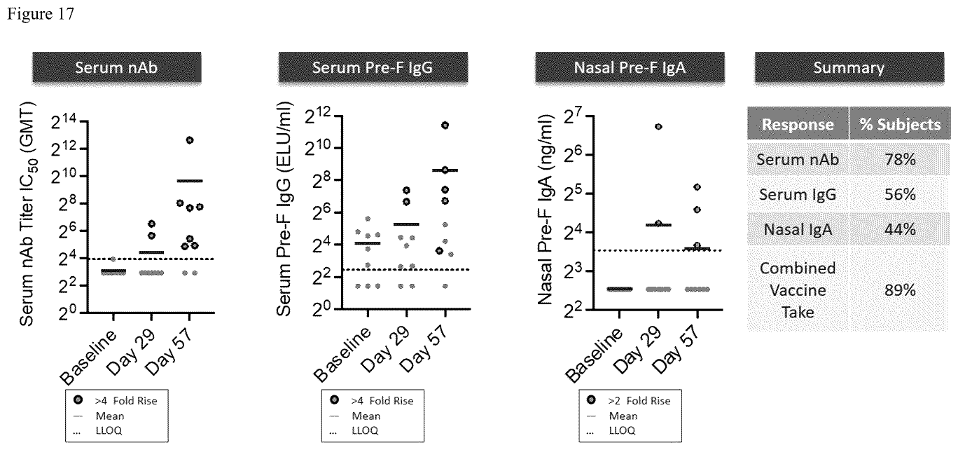

The change in serum binding (RSV F-specific) IgG concentrations were measured per participant at baseline, Day 27, and Day 57. In addition, the change in nasal mucosal binding (RSV F-specific) IgA concentrations was measured per participant at baseline, Day 29, and Day 57. FIG. 17 shows the serum nAb, serum pre-F IgG, and nasal pre-F IgA measured in subjects given two 10⁶ PFU doses given 29 days apart. A serum nAb response was detected in 78% of subjects, a serum IgG response was detected in 56% of subjects, and a nasal IgA response was detected in 44% of subjects, for a combined vaccine take of 89%.

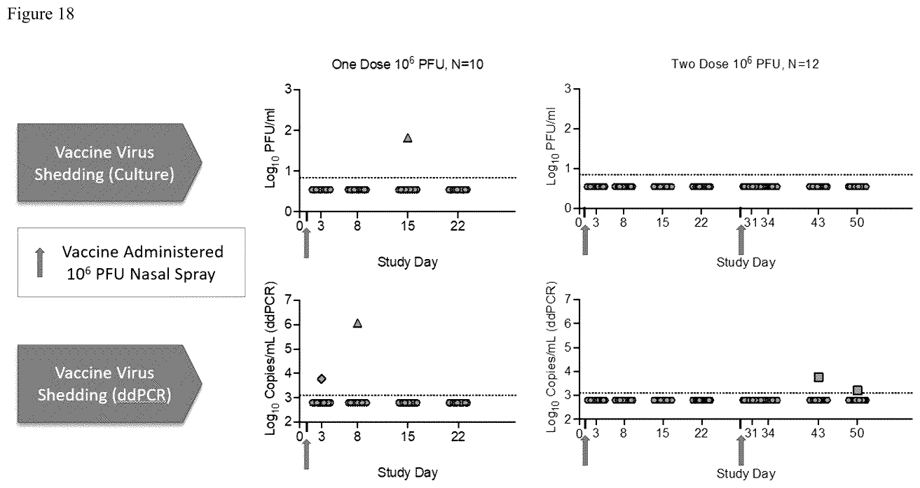

The frequency of any post-vaccination shedding of vaccine virus (as detected by plaque assay) after a single intranasal dose of MV-012-968 was measured per dosage group and overall from intranasal inoculation through Day 22 (an average of three (3) weeks). If post-vaccination shedding of vaccine virus was detected by plaque assay after a single intranasal dose of MV-012-968, peak viral titer (measured in plaque forming units, PFU) was measured per dosage group and overall from intranasal inoculation through Day 22 (an average of three (3) weeks). If post-vaccination shedding of vaccine virus was detected by plaque assay after a single intranasal dose of MV-012-968, the duration of shedding (in days) was measured per dosage group and overall from intranasal inoculation through Day 22 (an average of three (3) weeks). As shown in FIG. 18, negligible RSV vaccine shedding was observed, with only 3 out of 22 infants shedding vaccine virus as measured by plaque assay or PCR.

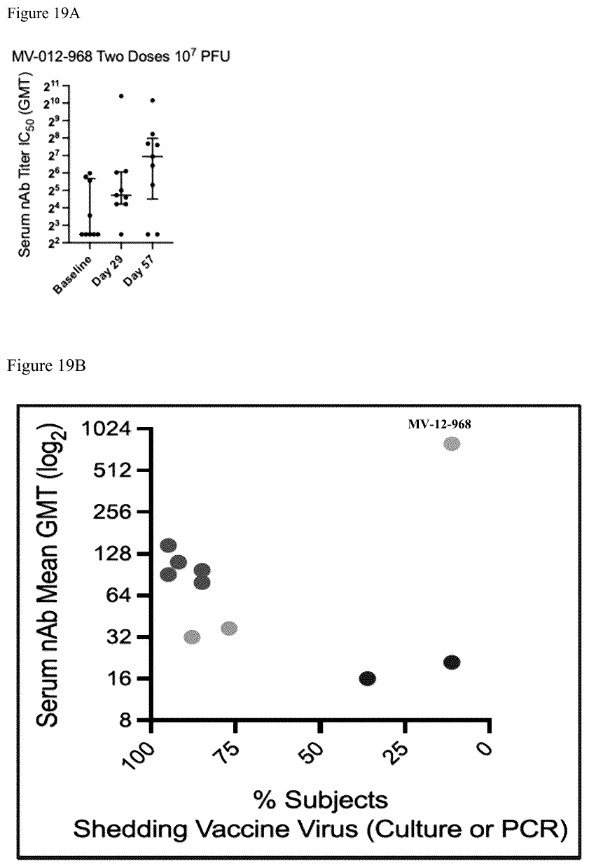

FIG. 19A shows the interim serum nAb results for 9 seronegative children (6-36 months of age) who received 2 doses at 10⁷ PFU of MV-012-968. The graph shows individual subjects, median, and interquartile range. As shown, administration of MV-012-968 leads to an increase in RSV-specific serum neutralizing antibody (nAb) titers.

FIG. 19B shows comparative safety, immunogenicity, and vaccine shedding data of different RSV live attenuated vaccines (LAV). Each dot represents a different RSV LAV candidate studied in seronegative infants and children in Phase 1. MV-012-968 Phase 1 data (green) are compared to prior, published candidates. Red, candidates with poor safety (e.g., LRI, cough). Orange, candidates with moderate safety (e.g., fever). Blue and green, candidates that were well-tolerated. Compared to previous RSV LAVs, two doses, 28 days apart, of 10⁶ PFU of MV-012-968 LAV was safe, immunogenic, with very low levels of shedding in seronegative pediatric subjects 6-36 months of age.

Other Pre-specified Outcome Measures being measured, include (1) frequency of RSV-confirmed medically attended (1) acute respiratory infection and (2) lower respiratory infection during peak RSV season following study inoculation are being measured, categorized by severity from approximately five (5) months duration during peak RSV season, adjusted for local epidemiology.

Eligibility Criteria for this Study
Ages Eligible for Study: 6 Months to 36 Months
Sexes Eligible for Study: All
Gender Based: No
Accepts Healthy Volunteers: Yes

### Inclusion Criteria:

Children aged 6-36 months
Good health based on history, physical examination, and medical record review, without evidence or suspicion of chronic disease
Seronegative to RSV, as defined by serum nAb titer below the threshold described in the study protocol and operations manual
Written informed consent provided by parent(s)/guardian(s)

### Exclusion Criteria:

Known or suspected chronic illness, particularly cardiopulmonary (including asthma or reactive airways disease), genetic or metabolic, hepatic, renal, infectious (including recurrent or chronic sinusitis), or immunodeficiency
Prior lab-confirmed RSV infection
Household or close contact (including but not limited to daycare) during the 21 days post-inoculation with anyone <6 months old or immunocompromised (applies to first study inoculation)
Nasal obstruction (including due to anatomic/structural causes, acute or chronic rhinosinusitis, or other causes)
Receipt of immunoglobulins, monoclonal antibodies and/or any blood products, or ribavirin within 6 months prior to study inoculation, or planned use during study period
Receipt of an investigational RSV vaccine at any time
Any other condition that, in the judgment of the investigator, would be a risk to subject's safety and/or may interfere with study procedures or interpretation of results

The Example demonstrates that subjects inoculated with MV-012-968 exhibit an increase RSV-specific serum neutralizing antibody (nAb) titers, an increase in serum binding (RSV F-specific) IgG concentrations, and an increase in nasal mucosal binding (RSV F-specific) IgA concentrations. It is expected that the subjects will exhibit a reduced frequency of RSV-confirmed medically attended (1) acute respiratory infection and (2) lower respiratory infection.

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

MV-012-968_Ref_Seq_001
SEQ ID NO: 22
ACGCGAAAAAATGCGTACAACAAACTTGCATAAACCAAAAAAATGGGGCAAATAAGAATTTGATAAGTACCACTTAAATTT
AACTCCCTTGCTTAGCGATGGGTTCGAATTCGCTATCGATGATAAAAGTACGTCTACAAAATCTATTTGATAATGATGAAG
TAGCGCTACTAAAAATAACGTGTTATACGGATAAACTAATACATCTAACGAATGCGCTAGCGAAAGCGGTAATACATACGA
TAAAACTAAATGGTATAGTATTTGTACATGTAATAACGTCGTCGGATATATGTCCGAATAATAATATAGTAGTAAAATCGA
ATTTTACGACGATGCCGGTACTACAAAATGGTGGTTATATATGGGAAATGATGGAACTAACGCATTGTTCGCAACCGAATG
GTCTACTAGATGATAATTGTGAAATAAAATTTTCGAAAAAACTATCGGATTCGACGATGACGAATTATATGAATCAACTAT
CGGAACTACTAGGTTTTGATCTAAATCCGTAAATTATAATTAATATCAACTAGCAAATCAATGTCACTAACACCATTAGTT
AATATAAAACTTAACAGAAGACAAAAATGGGGCAAATAAATCAATTCAGCCAACCCAACCATGGATACGACGCATAATGAT
AATACGCCGCAACGTCTAATGATAACGGATATGCGTCCGCTATCGCTAGAAACGATAATAACGTCGCTAACGCGTGATATA
ATAACGCATAAATTTATATATCTAATAAATCATGAATGTATAGTACGTAAACTAGATGAACGTCAAGCGACGTTTACGTTT
CTAGTAAATTATGAAATGAAACTACTACATAAAGTAGGTTCGACGAAATATAAAAAATATACGGAATATAATACGAAATAT
GGTACGTTTCCGATGCCGATATTTATAAATCATGATGGTTTTCTAGAATGTATAGGTATAAAACCGACGAAACATACGCCG
ATAATATATAAATATGATCTAAATCCGTAAATTTCAACACAATATTCACACAATCTAAAACAACAACTCTATGCATAACTA
TACTCCATAGTCCAGATGGAGCCTGAAAATTATAGTAATTTAAAATTAAGGAGAGATATAAGATAGAAGATGGGGCAAATA
CAAAGATGGCTCTTAGCAAAGTCAAGTTGAATGATACACTCAACAAAGATCAACTTCTGTCATCCAGCAAATACACCATCC
AACGGAGCACAGGAGATAGTATTGATACTCCTAATTATGATGTGCAGAAACACATCAATAAGTTATGTGGCATGTTATTAA
TCACAGAAGATGCTAATCATAAATTCACTGGGTTAATAGGTATGTTATATGCGATGTCTAGGTTAGGAAGAGAAGACACCA
TAAAAATACTCAGAGATGCGGGATATCATGTAAAAGCAAATGGAGTAGATGTAACAACACATCGTCAAGACATTAATGGAA
AAGAAATGAAATTTGAAGTGTTAACATTGGCAAGCTTAACAACTGAAATTCAAATCAACATTGAGATAGAATCTAGAAAAT
CCTACAAAAAAATGCTAAAAGAAATGGGAGAGGTAGCTCCAGAATACAGGCATGACTCTCCTGATTGTGGGATGATAATAT
TATGTATAGCAGCATTAGTAATAACTAAATTAGCAGCAGGGGACAGATCTGGTCTTACAGCCGTGATTAGGAGAGCTAATA
ATGTCCTAAAAAATGAAATGAAACGTTACAAAGGCTTACTACCCAAGGACATAGCCAACAGCTTCTATGAAGTGTTTGAAA
AACATCCCCACTTTATAGATGTTTTTGTTCATTTTGGTATAGCACAATCTTCTACCAGAGGTGGCAGTAGAGTTGAAGGGA
TTTTTGCAGGATTGTTTATGAATGCCTATGGTGCAGGGCAAGTGATGTTACGGTGGGGAGTCTTAGCAAAATCAGTTAAAA
ATATTATGTTAGGACATGCTAGTGTGCAAGCAGAAATGGAACAAGTTGTTGAGGTTTATGAATATGCCCAAAAATTGGGTG
GTGAAGCAGGATTCTACCATATATTGAACAACCCAAAAGCATCATTATTATCTTTGACTCAATTTCCTCACTTCTCCAGTG
TAGTATTAGGCAATGCTGCTGGCCTAGGCATAATGGGAGAGTACAGAGGTACACCGAGGAATCAAGATCTATATGATGCAG
CAAAGGCATATGCTGAACAACTCAAAGAAAATGGTGTGATTAACTACAGTGTACTAGACTTGACAGCAGAAGAACTAGAGG
CTATCAAACATCAGCTTAATCCAAAAGATAATGATGTAGAGCTTTGAGTTAATAAAAAATGGGGCAAATAAATCATCATGG
AAAAGTTTGCTCCTGAATTCCATGGAGAAGATGCAAACAACAGGGCTACTAAATTCCTAGAATCAATAAAGGGCAAATTCA
CATCACCCAAAGATCCCAAGAAAAAAGATAGTATCATATCTGTCAACTCAATAGATATAGAAGTAACCAAAGAAAGCCCTA
TAACATCAAATTCAACTATTATCAACCCAACAAATGAGACAGATGATACTGCAGGGAACAAGCCCAATTATCAAAGAAAAC
CTCTAGTAAGTTTCAAAGAAGACCCTACACCAAGTGATAATCCCTTTTCTAAACTATACAAAGAAACCATAGAAACATTTG
ATAACAATGAAGAAGAATCCAGCTATTCATACGAAGAAATAAATGATCAGACAAACGATAATATAACAGCAAGATTAGATA
GGATTGATGAAAAATTAAGTGAAATACTAGGAATGCTTCACACATTAGTAGTGGCAAGTGCAGGACCTACATCTGCTCGGG
ATGGTATAAGAGATGCCATGATTGGTTTAAGAGAAGAAATGATAGAAAAAATCAGAACTGAAGCATTAATGACCAATGACA
GATTAGAAGCTATGGCAAGACTCAGGAATGAGGAAAGTGAAAAGATGGCAAAAGACACATCAGATGAAGTGTCTCTCAATC
CAACATCAGAGAAATTGAACAACCTATTGGAAGGGAATGATACTGACAATGATCTATCACTTGAAGATTTCTGATTAGTTA
CCACTCTTCACATCAACACACAATACCAACAGAAGACCAACAAACTAACCAACCCAATCATCCAACCAAACATCCATCCCC
CAATCAGCCAAACAGCCAACAAAACAACCAGCCAATCCAAAACTAACCACCCGGAAAAAATCTATAATATAGTTACAAAAA
AAGGAAAGGGTGGGGCAAATATGGAAACATACGTGAACAAGCTTCACGAAGGCTCCACATACACAGCTGCTGTTCAATACA
ATGTCTTAGAAAAAGACGATGACCCTGCATCACTTACAATATGGGTGCCCATGTTCCAATCATCTATGCCAGCAGATTTAC
TTATAAAAGAACTAGCTAATGTCAACATACTAGTGAAACAAATATCCACACCCAAGGGACCTTCACTAAGAGTCATGATAA
ACTCAAGAAGTGCAGTGCTAGCACAAATGCCCAGCAAATTTACCATATGCGCTAATGTGTCCTTGGATGAAAGAAGCAAAC
TAGCATATGATGTAACCACACCCTGTGAAATCAAGGCATGTAGTCTAACATGCCTAAAATCAAAAAATATGTTGACTACAG
TTAAAGATCTCACTATGAAGACACTCAACCCTACACATGATATTATTGCTTTATGTGAATTTGAAAACATAGTAACATCAA
AAAAAGTCATAATACCAACATACCTAAGATCCATCAGTGTCAGAAATAAAGATCTGAACACACTTGAAAATATAACAACCA
CTGAATTCAAAAATGCTATCACAAATGCAAAAATCATCCCTTACTCAGGATTACTATTAGTCATCACAGTGACTGACAACA
AAGGAGCATTCAAATACATAAAGCCACAAAGTCAATTCATAGTAGATCTTGGAGCTTACCTAGAAAAAGAAAGTATATATT
ATGTTACCACAAATTGGAAGCACACAGCTACACGATTTGCAATCAAACCCATGGAAGATTAACCTTTTTCCTCTACATCAG
TGTGTTAATTCATACAAACTTTCTACCTACATTCTTCACTTCACCATCACAATCACAAACACTCTGTGGTTCAACCAATCA
AACAAAACTTATCTGAAGTCCCAGATCATCCCAAGTCATTGTTTATCAGATCTAGTACTCAAATAAGTTAATAAAAAATAT
ACACATGGACGTCCATGGGGCAAATGCAAACATGTCGAAAAATAAAGATCAACGTACGGCGAAAACGCTAGAACGTACGTG
GGATACGCTAAATCATCTACTATTTATATCGTCGTGTCTATATAAACTAAATCTAAAATCGGTAGCGCAAATAACGCTATC
GATACTAGCGATAATAATATCGACTTCGCTAATAATAGCGGCGATAATATTTATAGCGTCGGCGAATCATAAAGTAACGCC
GACGACGGCGATAATACAAGATGCGACTTCGCAAATAAAAAATACGACGCCGACGTATCTAACGCAAAATCCGCAACTAGG
TATATCGCCGTCGAATCCGTCGGAAATAACGTCGCAAATAACGACGATACTAGCGTCGACGACGCCGGGTGTAAAATCGAC
GCTACAATCGACGACGGTAAAAACGAAAAATACGACGACGACGCAAACGCAACCGTCGAAACCGACGACGAAACAACGTCA
AAATAAACCGCCGTCGAAACCGAATAATGATTTTCATTTTGAAGTATTTAATTTTGTACCGTGTTCGATATGTTCGAATAA
TCCGACGTGTTGGGCGATATGTAAACGTATACCGAATAAAAAACCGGGTAAAAAAACGACGACGAAACCGACGAAAAAACC
GACGCTAAAAACGACGAAAAAAGATCCGAAACCGCAAACGACGAAATCGAAAGAAGTACCGACGACGAAACCGACGGAAGA
ACCGACGATAAATACGACGAAAACGAATATAATAACGACGCTACTAACGTCGAATACGACGGGTAATCCGGAACTAACGTC
GCAAATGGAAACGTTTCATTCGACTTCGTCGGAAGGTAATCCGTCGCCGTCGCAAGTATCGACGACTTCGGAATATCCGTC
GCAACCGTCGTCGCCGCCGAATACGCCGCGTCAATAGTTACTTAAAAACATATTATCACAAAAGGCCTTGACCAACCGCGG
AGAATCAAAATAAACTCTGGGGCAAATAACAATGGAGTTGCCAATCCTCAAAGCAAATGCAATTACCACAATCCTCGCTGC
AGTCACATTTTGCTTTGCTTCTAGTCAAAACATCACTGAAGAATTTTATCAATCAACATGCAGTGCAGTTAGCAAAGGCTA
TCTTAGTGCTCTAAGAACTGGTTGGTATACTAGTGTTATAACTATAGAATTAAGTAATATCAAGAAAAATAAGTGTAATGG
AACAGATGCTAAGGTAAAATTGATGAAACAAGAATTAGATAAATATAAAAATGCTGTAACAGAATTGCAGTTGCTCATGCA
AAGCACACCAGCAGCAAACAATCGAGCCAGAAGAGAACTACCAAGGTTTATGAATTATACACTCAACAATACCAAAAAAAC
CAATGTAACATTAAGCAAGAAAAGGAAAAGAAGATTTCTTGGTTTTTTGTTAGGTGTTGGATCTGCAATCGCCAGTGGCAT
TGCTGTATCTAAGGTCCTGCACTTAGAAGGAGAAGTGAACAAGATCAAAAGTGCTCTACTATCCACAAACAAGGCCGTAGT
CAGCTTATCAAATGGAGTTAGTGTCTTAACCAGCAGAGTGTTAGACCTCAAAAACTATATAGATAAACAATTGTTACCTAT
TGTGAATAAGCAAAGCTGCAGAATATCAAATATAGAAACTGTGATAGAGTTCCAACAAAAGAACAACAGACTACTAGAGAT
TACCAGGGAATTTAGTGTTAATGCAGGTGTAACTACACCTGTAAGCACTTACATGTTAACTAATAGTGAATTATTGTCATT
AATCAATGATATGCCTATAACAAATGATCAGAAAAAGTTAATGTCCAACAATGTTCAAATAGTTAGACAGCAAAGTTACTC
TATCATGTCCATAATAAAAGAGGAAGTCTTAGCATATGTAGTACAATTACCACTATATGGTGTGATAGATACACCTTGTTG
GAAATTACACACATCCCCTCTATGTACAACCAACACAAAAGAAGGGTCAAACATCTGTTTAACAAGAACTGACAGAGGATG
GTACTGTGACAATGCAGGATCAGTATCTTTCTTCCCACAAGCTGAAAAATGTAAAGTTCAATCGAATCGAGTATTTTGTGA
CACAATGTACAGTTTAACATTACCAAGTGAAGTAAATCTCTGCAATGTTGACATATTCAATCCCAAATATGATTGTAAAAT
TATGACTTCAAAAACAGATGTAAGCAGCTCCGTTATCACATCTCTAGGAGCCATTGTGTCATGCTATGGCAAAACTAAATG
TACAGCATCCAATAAAAATCGTGGAATCATAAAGACATTTTCTAACGGGTGTGATTATGTATCAAATAAAGGGGTGGACAC
TGTGTCTGTAGGTAACACATTATATTATGTAAATAAGCAAGAAGGCAAAAGTCTCTATGTAAAAGGTGAACCAATAATAAA
TTTCTATGACCCATTAGTATTCCCCTCTGATGAATTTGATGCATCAATATCTCAAGTCAATGAGAAGATTAACCAGAGTTT
AGCATTTATTCGTAAATCCGATGAATTATTACATAATGTAAATGCTGGTAAATCAACCACAAATATCATGATAACTACTAT
AATTATAGTGATTATAGTAATATTGTTATCATTAATTGCTGTTGGACTGCTCCTATACTGTAAGGCCAGAAGCACACCAGT
CACACTAAGCAAGGATCAACTGAGTGGTATAAATAATATTGCATTTAGTAACTGAATAAAAATAGCACCTAATCATGTTCT
TACAATGGTTTACTATCTGCTCATAGACAACCCATCTATCATTGGATTTTCTTAAAATCTGAACTTCATCGAAACTCTTAT
CTATAAACCATCTCACTTACACTATTTAAGTAGATTCCTAGTTTATAGTTATATAAAACACAATTGAATGCCAGTCGACCT
TACCATCTGTAAAAATGAAAACTGGGGCAAATATGTCACGAAGGAATCCTTGCAAATTTGAAATTCGAGGTCATTGCTTAA
ATGGTAAGAGGTGTCATTTTAGTCATAATTATTTTGAATGGCCACCCCATGCACTGCTTGTAAGACAAAACTTTATGTTAA
ACAGAATACTTAAGTCTATGGATAAAAGTATAGATACCTTATCAGAAATAAGTGGAGCTGCAGAGTTGGACAGAACAGAAG
AGTATGCTCTTGGTGTAGTTGGAGTGCTAGAGAGTTATATAGGATCAATAAACAATATAACTAAACAATCAGCATGTGTTG
CCATGAGCAAACTCCTCACTGAACTCAATAGTGATGATATCAAAAAGCTGAGGGACAATGAAGAGCTAAATTCACCCAAGA
TAAGAGTGTACAATACTGTCATATCATATATTGAAAGCAACAGGAAAAACAATAAACAAACTATCCATCTGTTAAAAAGAT
TGCCAGCAGACGTATTGAAGAAAACCATCAAAAACACATTGGATATCCATAAGAGCATAACCATCAACAACCCAAAAGAAT
CAACTGTTAGTGATACAAATGACCATGCCAAAAATAATGATACTACCTGACAAATATCCTTGTAGTATAACTTCCATACTA
ATAACAAGTAGATGTAGAGTTACTATGTATAATCAAAAGAACACACTATATTTCAATCAAAACAACCCAAATAACCATATG
TACTCACCGAATCAAACATTCAATGAAATCCATTGGACCTCTCAAGAATTGATTGACACAATTCAAAATTTTCTACAACAT
CTAGGTATTATTGAGGATATATATACAATATATATATTAGTGTCATAACACTCAATTCTAACACTCACCACATCGTTACAT
TATTAATTCAAACAATTCAAGTTGTGGGACAAAATGGATCCCATTATTAATGGAAATTCTGCTAATGTTTATCTAACCGAT
AGTTATTTAAAAGGTGTTATCTCTTTCTCAGAGTGTAATGCTTTAGGAAGTTACATATTCAATGGTCCTTATCTCAAAAAT
GATTATACCAACTTAATTAGTAGACAAAATCCATTAATAGAACACATGAATCTAAAGAAACTAAATATAACACAGTCCTTA
ATATCTAAGTATCATAAAGGTGAAATAAAATTAGAAGAACCTACTTATTTTCAGTCATTACTTATGACATACAAGAGTATG
ACCTCGTCAGAACAGATTGCTACCACTAATTTACTTAAAAAGATAATAAGAAGAGCTATAGAAATAAGTGATGTCAAAGTC
TATGCTATATTGAATAAACTAGGGCTTAAAGAAAAGGACAAGATTAAATCCAACAATGGACAAGATGAAGACAACTCAGTT
ATTACGACCATAATCAAAGATGATATACTTTCAGCTGTTAAAGATAATCAATCTCATCTTAAAGCAGACAAAAATCACTCT
ACAAAACAAAAAGACACAATCAAAACAACACTCTTGAAGAAATTGATGTGTTCAATGCAACATCCTCCATCATGGTTAATA
CATTGGTTTAACTTATACACAAAATTAAACAACATATTAACACAGTATCGATCAAATGAGGTAAAAAACCATGGGTTTACA
TTGATAGATAATCAAACTCTTAGTGGATTTCAATTTATTTTGAACCAATATGGTTGTATAGTTTATCATAAGGAACTCAAA
AGAATTACTGTGACAACCTATAATCAATTCTTGACATGGAAAGATATTAGCCTTAGTAGATTAAATGTTTGTTTAATTACA
TGGATTAGTAACTGCTTGAACACATTAAATAAAAGCTTAGGCTTAAGATGCGGATTCAATAATGTTATCTTGACACAACTA
TTCCTTTATGGAGATTGTATACTAAAGCTATTTCACAATGAGGGGTTCTACATAATAAAAGAGGTAGAGGGATTTATTATG
TCTCTAATTTTAAATATAACAGAAGAAGATCAATTCAGAAAACGATTTTATAATAGTATGCTCAACAACATCACAGATGCT
GCTAATAAAGCTCAGAAAAATCTGCTATCAAGAGTATGTCATACATTATTAGATAAGACAGTGTCCGATAATATAATAAAT
GGCAGATGGATAATTCTATTAAGTAAGTTCCTTAAATTAATTAAGCTTGCAGGTGACAATAACCTTAACAATCTGAGTGAA
CTATATTTTTTGTTCAGAATATTTGGACACCCAATGGTAGATGAAAGACAAGCCATGGATGCTGTTAAAATTAATTGCAAT
GAGACCAAATTTTACTTGTTAAGCAGTCTGAGTATGTTAAGAGGTGCCTTTATATATAGAATTATAAAAGGGTTTGTAAAT
AATTACAACAGATGGCCTACTTTAAGAAATGCTATTGTTTTACCCTTAAGATGGTTAACTACTATAAACTAAACACTTTAT
CCTTCTTTGTTGGAACTTACAGAAAGAGATTTGATTGTGTTATCAGGACTACGTTTCTATCGTGAGTTTCGGTTGCCTAAA
AAAGTGGATCTTGAAATGATTATAAATGATAAAGCTATATCACCTCCTAAAAATTTGATATGGACTAGTTTCCCTAGAAAT
TACATGCCATCACACATACAAAACTATATAGAACATGAAAAATTAAAATTTTCCGAGAGTGATAAATCAAGAAGAGTATTA
GAGTATTATTTAAGAGATAACAAATTCAATGAATGTGATTTATACAACTGTGTAGTTAATCAAAGTTATCTCAACAACCCT
AATCATGTGGTATCATTGACAGGCAAAGAAAGAGAACTCAGTGTAGGTAGAATGTTTGCAATGCAACCGGGAATGTTCAGA
CAGGTTCAAATATTGGCAGAGAAAATGATAGCTGAAAACATTTTACAATTCTTTCCTGAAAGTCTTACAAGATATGGTGAT
CTAGAACTACAAAAAATATTAGAATTGAAAGCAGGAATAAGTAACAAATCAAATCGCTACAATGATAATTACAACAATTAC
ATTAGTAAGTGCTCTATCATCACAGATCTCAGCAAATTCAATCAAGCATTTCGATATGAAACGTCATGTATTTGTAGTGAT
GTGCTGGATGAACTGCATGGTGTACAATCTCTATTTTCCTGGTTACATTTAACTATTCCTCATGTCACAATAATATGCACA
TATAGGCATGCACCCCCCTATATAGGAGATCATATTGTAGATCTTAACAATGTAGATGAACAAAGTGGATTATATAGATAT
CACATGGGTGGCATCGAAGGGTGGTGTCAAAAACTGTGGACCATAGAAGCTATATCACTATTGGATCTAATATCTCTCAAA
GGGAAATTCTCAATTACTGCTTTAATTAATGGTGACAATCAATCAATAGATATAAGCAAACCAATCAGACTCATGGAAGGT
CAAACTCATGCTCAAGCAGATTATTTGCTAGCATTAAATAGCCTTAAATTACTGTATAAAGAGTATGCAGGCATAGGCCAC
AAATTAAAAGGAACTGAGACTTATATATCACGAGATATGCAATTTATGAGTAAAACAATTCAACATAACGGTGTATATTAC
CCAGCTAGTATAAAGAAAGTCCTAAGAGTGGGACCGTGGATAAACACTATACTTGATGATTTCAAAGTGAGTCTAGAATCT
ATAGGTAGTTTGACACAAGAATTAGAATATAGAGGTGAAAGTCTATTATGCAGTTTAATATTTAGAAATGTATGGTTATAT
AATCAGATTGCTCTACAATTAAAAAATCATGCATTATGTAACAATAAACTATATTTGGACATATTAAAGGTTCTGAAACAC
TTAAAAACCTTTTTTAATCTTGATAATATTGATACAGCATTAACATTGTATATGAATTTACCCATGTTATTTGGTGGTGGT
GATCCCAACTTGTTATATCGAAGTTTCTATAGAAGAACTCCTGACTTCCTCACAGAGGCTATAGTTCACTCTGTGTTCATA
CTTAGTTATTATACAAACCATGACTTAAAAGATAAACTTCAAGATCTGTCAGATGATAGATTGAATAAGTTCTTAACATGC
ATAATCACGTTTGACAAAAACCCTAATGCTGAATTCGTAACATTGATGAGAGATCCTCAAGCTTTAGGGTCTGAGAGACAA
GCTAAAATTACTAGCGAAATCAATAGACTGGCAGTTACAGAGGTTTTGAGTACAGCTCCAAACAAAATATTCTCCAAAAGT
GCACAACATTATACTACTACAGAGATAGATCTAAATGATATTATGCAAAATATAGAACCTACATATCCTCATGGGCTAAGA
GTTGTTTATGAAAGTTTACCCTTTTATAAAGCAGAGAAAATAGTAAATCTTATATCAGGTACAAAATCTATAACTAACATA
CTGGAAAAAACTTCTGCCATAGACTTAACAGATATTGATAGAGCCACTGAGATGATGAGGAAAAACATAACTTTGCTTATA
AGGATACTTCCATTGGATTGTAACAGAGATAAAAGAGAGATATTGAGTATGGAAAACCTAAGTATTACTGAATTAAGCAAA
TATGTTAGGGAAAGATCTTGGTCTTTATCCAATATAGTTGGTGTTACATCACCCAGTATCATGTATACAATGGACATCAAA
tATACTACAAGCACTATATCTAGTGGCATAATTATAGAGAAATATAATGTTAACAGTTTAACACGTGGTGAGAGAGGACCC
ACTAAACCATGGGTTGGTTCATCTACACAAGAGAAAAAAACAATGCCAGTTTATAATAGACAAGTCTTAACCAAAAAACAG
AGAGATCAAATAGATCTATTAGCAAAATTGGATTGGGTGTATGCATCTATAGATAACAAGGATGAATTCATGGAAGAACTC
AGCATAGGAACCCTTGGGTTAACATATGAAAAGGCCAAGAAATTATTTCCACAATATTTAAGTGTCAATTATTTGCATCGC
CTTACAGTCAGTAGTAGACCATGTGAATTCCCTGCATCAATACCAGCTTATAGAACAACAAATTATCACTTTGACACTAGC
CCTATTAATCGCATATTAACAGAAAAGTATGGTGATGAAGATATTGACATAGTATTCCAAAACTGTATAAGCTTTGGCCTT
AGTTTAATGTCAGTAGTAGAACAATTTACTAATGTATGTCCTAACAGAATTATTCTCATACCTAAGCTTAATGAGATACAT
TTGATGAAACCTCCCATATTCACAGGTGATGTTGATATTCACAAGTTAAAACAAGTGATACAAAAACAGCATATGTTTTTA
CCAGACAAAATAAGTTTGACTCAATATGTGGAATTATTCTTAAGTAATAAAACACTCAAATCTGGATCTCATGTTAATTCT
AATTTAATATTGGCACATAAAATATCTGACTATTTTCATAATACTTACATTTTAAGTACTAATTTAGCTGGACATTGGATT
CTGATTATACAACTTATGAAAGATTCTAAAGGTATTTTTGAAAAAGATTGGGGAGAGGGATATATAACTGATCATATGTTT
ATTAATTTGAAAGTTTTCTTCAATGCTTATAAGACCTATCTCTTGTGTTTTCATAAAGGTTATGGCAAAGCAAAGCTGGAG
TGTGATATGAACACTTCAGATCTTCTATGTGTATTGGAATTAATAGACAGTAGTTATTGGAAGTCTATGTCTAAGGTATTT
TTAGAACAAAAAGTTATCAAATACATTCTTAGCCAAGATGCAAGTTTACATAGAGTAAAAGGATGTCATAGCTTCAAATTA
TGGTTTCTTAAACGTCTTAATGTAGCAGAATTCACAGTTTGCCCTTGGGTTGTTAACATAGATTATCATCCAACACATATG
AAAGCAATATTAACTTATATAGATCTTGTTAGAATGGGATTGATAAATATAGATAGAATACACATTAAAAATAAACACAAA
TTCAATGATGAATTTTATACTTCTAATCTCTTCTACATTAATTATAACTTCTCAGATAATACTCATCTATTAACTAAACAT
ATAAGGATTGCTAATTCTGAATTAGAAAATAATTACAACAAATTATATCATCCTACACCAGAAACCCTAGAGAATATACTA
GCCAATCCGATTAAAAGTAATGACAAAAAGACACTGAATGACTATTGTATAGGTAAAAATGTTGACTCAATAATGTTACCA
TTGTTATCTAATAAGAAGCTTATTAAATCGTCTGCAATGATTAGAACCAATTACAGCAAACAAGATTTGTATAATTTATTC
CCTATGGTTGTGATTGATAGAATTATAGATCATTCAGGCAATACAGCCAAATCCAACCAACTTTACACTACTACTTCCCAC
CAAATATCTTTAGTGCACAATAGCACATCACTTTACTGCATGCTTCCTTGGCATCATATTAATAGATTCAATTTTGTATTT
AGTTCTACAGGTTGTAAAATTAGTATAGAGTATATTTTAAAAGATCTTAAAATTAAAGATCCCAATTGTATAGCATTCATA
GGTGAAGGAGCAGGGAATTTATTATTGCGTACAGTAGTGGAACTTCATCCTGACATAAGATATATTTACAGAAGTCTGAAA
GATTGCAATGATCATAGTTTACCTATTGAGTTTTTAAGGCTGTACAATGGACATATCAACATTGATTATGGTGAAAATTTG
ACCATTCCTGCTACAGATGCAACCAACAACATTCATTGGTCTTATTTACATATAAAGTTTGCTGAACCTATCAGTCTTTTT
GTCTGTGATGCCGAATTGTCTGTAACAGTCAACTGGAGTAAAATTATAATAGAATGGAGCAAGCATGTAAGAAAGTGCAAG
TACTGTTCCTCAGTTAATAAATGTATGTTAATAGTAAAATATCATGCTCAAGATGATATTGATTTCAAATTAGACAATATA
ACTATATTAAAAACTTATGTATGCTTAGGCAGTAAGTTAAAGGGATCGGAGGTTTACTTAGTCCTTACAATAGGTCCTGCG
AATATATTCCCAGTATTTAATGTAGTACAAAATGCTAAATTGATACTATCAAGAACCAAAAATTTCATCATGCCTAAGAAA
GCTGATAAAGAGTCTATTGATGCAAATATTAAAAGTTTGATACCCTTTCTTTGTTACCCTATAACAAAAAAAGGAATTAAT
ACTGCATTGTCAAAACTAAAGAGTGTTGTTAGTGGAGATATACTATCATATTCTATAGCTGGACGTAATGAAGTTTTCAGC
AATAAACTTATAAATCATAAGCATATGAACATCTTAAAATGGTTCAATCATGTTTTAAATTTCAGATCAACAGAACTAAAC
TATAACCATTTATATATGGTAGAATCTACATATCCTTACCTAAGTGAATTGTTAAACAGCTTGACAACCAATGAACTTAAA
AAACTGATTAAAATCACAGGTAGTCTGTTATACAACTTTCATAATGAATAATGAATAAAGATCTTATAATAAAAATTCCCA
TAGCTATACACTAACACTGTATTCAATTATAGTTATTAAAAATTAAAAATCGTACGATTTTTTAAATAACTTTTAGTGAAC
TAATCCTAAAGTTATCATTTTAATCTTGGAGGAATAAATTTAAACCCTAATCTAATTGGTTTATATGTGTATTAACTAAAT
TACGAGATATTAGTTTTTGACACTTTTTTTCTCGT

1. A method of administering a live attenuated respiratory syncytial virus (RSV) vaccine to a subject in need thereof, the method comprising:
intranasally administering the live attenuated RSV vaccine in an amount of from about 10³ pfu/dose to about 10⁷ pfu per dose to the subject, wherein the subject is a child between 6 and 24 months of age or between about 6 and 36 months of age, wherein administration of the live attenuated RSV vaccine results in at least one of (a) an increase in RSV-specific serum neutralizing antibody (nAb) titers in the subject, (b) an increase in serum binding (RSV F-specific) IgG concentrations in the subject, and (c) an increase in nasal mucosal binding (RSV F-specific) IgA concentrations in the subject, and wherein the live attenuated RSV vaccine comprises a genome comprising one or more of SEQ ID NOs: 6, 9, and 20.

2. The method of claim 1, wherein the administration results in a reduced likelihood or severity of infection by RSV.

3. The method of claim 1, wherein the administration is in a single dose of about 10³ pfu/dose.

4. The method of claim 1, wherein the administration is in a single dose of about 10⁴ pfu/dose.

5. The method of claim 1, wherein the administration is in a single dose of about 10⁵ pfu/dose.

6. The method of claim 1, wherein the administration is in a single dose of about 10⁶ pfu/dose.

7. The method of claim 1, wherein the administration is in a single dose of about 10⁴ pfu/dose.

8. The method of claim 1, wherein the administration is in two doses of about 10³ pfu/dose to about 10⁷ pfu/dose.

9. The method of claim 1, wherein the administration is in two doses of about 10⁴ pfu/dose.

10. The method of claim 1, wherein the administration is in two doses of about 10⁵ pfu/dose.

11. The method of claim 1, wherein the administration is in two doses of about 10⁶ pfu/dose.

12. The method of claim 1, wherein the administration is in two doses of about 10⁷ pfu/dose.

13. The method of claim 7, wherein the two doses are administered between about 3 and about 6 weeks apart.

14. The method of claim 7, wherein the two doses are administered between about 4 and about 5 weeks apart.

15. The method of claim 7, wherein the two doses are administered between about 28, 29, or 30 days apart.

16. The method of claim 1, wherein administration of the live attenuated RSV vaccine results in an increase in RSV-specific serum neutralizing antibody (nAb) titers in the subject.

17. The method of claim 1, wherein administration of the live attenuated RSV vaccine results in an increase in serum binding (RSV F-specific) IgG concentrations in the subject.

18. The method of claim 1, wherein administration of the live attenuated RSV vaccine results in an increase in nasal mucosal binding (RSV F-specific) IgA concentrations in the subject.

19. The method of claim 1, wherein the live attenuated RSV vaccine comprises SEQ ID NOs: 6, 9, and 20.

20. The method of claim 1, wherein the live attenuated RSV vaccine comprises SEQ ID NO: 22.

21. The method of claim 1, wherein the intranasal administration is by drops or by nasal atomizer.
